SEQUENCE LISTING (45 sequences)

SEQ ID NO: 1 - DNA, 1203 bp, Escherichia coli
atggcaattaaattagaaattaaaaatctttataaaatatttggcgagcatccacagcgagcgttcaaatatatcgaacaaggactttcaaaagaacaaattctggaaaaaactgggctatcgcttggcgtaaaagacgccagtctggccattgaagaaggcgagatatttgtcatcatgggattatccggctcgggtaaatccacaatggtacgccttctcaatcgcctgattgaacccacccgcgggcaagtgctgattgatggtgtggatattgccaaaatatccgacgccgaactccgtgaggtgcgcagaaaaaagattgcgatggtcttccagtcctttgccttaatgccgcatatgaccgtgctggacaatactgcgttcggtatggaattggccggaattaatgccgaagaacgccgggaaaaagcccttgatgcactgcgtcaggtcgggctggaaaattatgcccacagctacccggatgaactctctggcgggatgcgtcaacgtgtgggattagcccgcgcgttagcgattaatccggatatattattaatggacgaagccttctcggcgctcgatccattaattcgcaccgagatgcaggatgagctggtaaaattacaggcgaaacatcagcgcaccattgtctttatttcccacgatcttgatgaagccatgcgtattggcgaccgaattgccattatgcaaaatggtgaagtggtacaggtcggcacaccggatgaaattctcaataatccggcgaatgattatgtccgtaccttcttccgtggcgttgatattagtcaggtattcagtgcgaaagatattgcccgccggacaccgaatggcttaattcgtaaaacccctggcttcggcccacgttcggcactgaaattattgcaggatgaagatcgcgaatatggctacgttatcgaacgcggtaataagtttgtcggcgcagtctccatcgattcgcttaaaaccgcgttaacgcagcagcaaggtcttgatgcggcgctgattgatgcgccgttagcagtcgatgcacaaacgcctcttagcgagttgctctctcatgtcggacaggcaccctgtgcggtgcccgtggtcgacgaggaccaacagtatgtcggcatcatttcgaaaggaatgctgctgcgcgctttagatcgtgagggggtaaataatggctga

SEQ ID NO: 2 - DNA, 1065 bp, Escherichia coli
atggctgatcaaaataatccgtgggataccacgccagcggcggacagtgccgcgcaatccgcagacgcctggggtacaccgacgactgcaccgactgacggcggtggtgctgactggctgaccagtacgcctgcgccaaacgtcgagcattttaatattctcgatccgttccataaaacgctgatcccgctcgacagttgggtcactgaagggatcgactgggtcgttacccatttccgtcccgtcttccagggcgtgcgcgttccggttgattatatcctcaacggtttccagcaattgctgctgggtatgcccgcaccggtggcgattatcgttttcgctctcatcgcctggcagatttccggggtcggaatgggtgtggcgacgctggtttcgctgattgccatcggcgcaatcggtgcctggtcgcaggcaatggtgactctggcgctggtgttaaccgccctgctgttctgtatcgtcatcggtttgccgttggggatatggctggcgagaagtccgcgagcggcgaaaattattcgtccactgcttgatgccatgcagaccacgccagcgtttgtttatctggtgccaatcgtcatgctatttggtatcggtaacgtgccgggcgtggtggtgacgatcatctttgctctgccgccgattatccgtctgaccattctggggattaaccaggttccggcggatctgattgaagcctcgcgctcattcggtgccagcccgcgccagatgctgttcaaagttcagttaccgctggcgatgccgaccattatggcgggcgttaaccagacgctgatgctggccctttctatggtggtcatcgcctcgatgattgccgtcggcgggttgggtcagatggtacttcgcggtatcggtcgtctggatatggggcttgccaccgttggcggcgtcgggattgtgatcctcgccattatcctcgatcgtctgacgcaggccgttgggcgcgactcacgcagtcgcggcaaccgtcgctggtacaccactggccctgttggtctgctgacccgcccattcattaagtaa

SEQ ID NO: 3 - DNA, 993 bp, Escherichia coli
atgcgacatagcgtactttttgcgacagcgtttgccacgcttatctctacacaaacttttgctgccgatctgccgggcaaaggcattactgttaatccagttcagagcaccatcactgaagaaaccttccagacgctgctggtcagtcgtgcgctggagaaattaggttataccgtcaacaaacccagcgaagtagattacaacgttggctacacctcgcttgcttccggcgatgcaaccttcaccgccgtgaactggacgccactgcatgacaacatgtacgaagctgccggtggcgataagaaattttatcgtgaaggggtatttgttaacggcgcggcacagggttacctgatcgataagaaaaccgccgaccagtacaaaatcaccaacatcgcacaactgaaagatccgaagatcgccaaactgttcgataccaacggcgacggaaaagcggatttaaccggttgtaaccctggctggggctgcgaaggtgcgatcaaccaccagcttgccgcgtatgaactgaccaacaccgtgacgcataatcaggggaactacgcagcgatgatggccgacaccatcagtcgctacaaagagggcaaaccggtgttttattacacctggacgccgtactgggtgagtaacgaactgaagccgggcaaagatgtcgtctggttgcaggtgccgttctccgcactgccgggcgataaaaacgccgataccaaactgccgaatggtgcgaattatggcttcccggtcagcaccatgcatatcgttgccaacaaagcctgggccgagaaaaacccggcagcagcgaaactgtttgccattatgcagttgccagtggcagatattaacgcccagaacgccattatgcatgacggcaaagcctcagaaggcgatattcagggacacgttgatggttggatcaaagcccaccagcagcagttcgatggctgggtgaatgaggcgctggcagcgcagaagtaa

SEQ ID NO: 4 - DNA, 210 bp, Escherichia coli
atggcaaagattaaaggtcaggttaagtggttcaacgagtctaaaggttttggcttcattactccggctgatggcagcaaagatgtgttcgtacacttctccgctatccagggtaatggcttcaaaactctggctgaaggtcagaacgttgagttcgaaattcaggacggccagaaaggtccggcagctgttaacgtaacagctatctga

SEQ ID NO: 5 - DNA, 2247 bp, Escherichia coli
atgctcactcgcctgcgcgaaatagtcgaaaaggtagccagcgcaccacgcctgaatgaggcgttaaatattctggttaccgacatctgtcttgcgatggataccgaggtctgttcggtctacctggccgatcatgatcgacgttgttactacctgatggcgacccgggggctgaaaaaaccacgcggtcgcactgtaacgctcgcgtttgatgaagggatcgtcggcctggttggcaggctggcggaaccgataaaccttgcagatgcgcaaaagcaccccagcttcaaatacatcccctccgtaaaagaagaacgtttccgcgcgtttttaggcgtaccaattattcaacgtcgccagttgcttggtgtactggtggtacagcaacgagagttgcgccagtatgacgaaagtgaagaatccttcctggtgacgcttgccacccagatggcagctattctttctcagtcgcagttgactgccttgtttgggcaatatcgccagacgcgaatccgcgcattaccggcagcacctggtgtggcgattgccgaaggctggcaggatgccacgttacctttaatggaacaggtgtatcaggcatcaacgctggatccggctctggaacgcgaacgactgaccggggcgctggaagaagcggcaaacgagtttcgccgctacagcaaacgctttgccgccggtgcacaaaaagaaacggcggctattttcgatctttactcgcacctgctttcggacacccggctgcgtcgcgaattgtttgccgaggttgataaaggctcggtggcagagtgggcggtaaaaacggtcattgaaaaatttgccgaacagtttgccgcgctaagtgataactatctcaaagagcgggctggcgatttacgtgcgctgggtcagcgattgctgtttcatcttgatgacgctaatcaagggccgaacgcctggccggaacgtttcattctggtggcagatgaactgtcagcgacaacgcttgctgagctgccccaggatcgcttagtcggtgttgtcgtgcgagatggcgcagccaactcccatgctgcgatcatggtacgtgcgctggggatccctaccgtgatgggcgcggatattcagccttcggtgctgcatcgtaggacgctgatcgttgatggctatcgcggtgaattgctggtcgatccggagccggtactgctgcaagaatatcagcggctaattagtgaagagattgagcttagccgtctggcggaagatgacgtcaatttacccgcgcagttaaaaagcggtgagcgtataaaagtcatgctcaatgctggtttaagcccggaacatgaagaaaaactgggcagccgtattgatggcatcggtctttatcgcactgaaatcccattcatgctgcaaagtggtttcccgtcggaagaagaacaggtggcgcagtatcaggggatgctgcaaatgttcaatgataaacccgtcaccttgcgtacgctggatgtcggagcagataagcagctgccttacatgccgatcagcgaagagaatccatgcctgggttggcgtgggattcgcattacgctcgatcagccggagatcttcttgatccaggtgcgggcgatgctgcgtgctaatgccgctacgggcaacctgaatattctgttgccgatggtcacaagcctcgatgaagttgatgaagcacgccgcctgattgaacgtgccggacgtgaagtcgaggagatgatcggttacgaaattcccaaaccacgtatcggcatcatgctggaagtgccgtcaatggtatttatgctgccgcatctggcaaagcgggtcgatttcatctctgttggcaccaacgatctgactcaatacattctggccgttgatcgcaacaatacccgggtggcgaacatttatgacagtcttcatcctgcaatgttacgagctctggcgatgatcgcccgggaagcggaaatacatggaatcgatctccgtttgtgcggtgaaatggcgggcgatcccatgtgcgtggcaatcctcattgggcttgggtatcgccatctgtctatgaacggacgttctgtagcgcgggcaaaatacctgctgcggcgcattgattatgccgaagcagaaaatcttgcgcagcgtagtctggaagcgcaactggcgaccgaagttcgccatcaggttgcagcctttatggagcgtcgcggcatgggcgggctgattcgcggagggttatag

SEQ ID NO: 6 - DNA, 1119 bp, Escherichia coli
atgggaaaaagcatagtcgttgtttctgcggtcaattttaccactggcggtccatttaccattttgaaaaaatttttggcagcaactaataataaagaaaatgtcagttttatcgcattagtccattctgctaaagagttaaaagaaagttatccatgggttaaattcattgagtttcctgaggttaaagggtcgtggctaaaacgtttgcactttgaatatgtagtttgtaaaaaactttcaaaagagctgaatgctacgcattggatttgtctgcatgatattacggccaatgtcgtcactaaaaaaagatatgtgtattgtcataaccctgcacctttttataaaggaattttattccgtgaaattcttatggagcctagctttttcttatttaaaatgctatacgggctgatatataaaataaacattaaaaaaaatactgcagtgtttgttcaacaattctggatgaaagaaaaatttatcaagaaatattctataaataacatcattgtcagtcggccagaaattaaattatctgataaaagccaacttactgatgatgattctcaatttaagaataacccttctgagttgacaatattttaccctgctgttccacgagtatttaaaaattacgagcttattattagtgcagcaaggaaattgaaagaacaatccaatattaaatttctgcttactatcagtggtacagaaaatgcgtatgcaaaatatattatcagtcttgcagaaggactggataatgttcatttcctcgggtacttggataaagaaaaaatcgatcattgttataatatttcagatatagtttgttttccctctaggttagaaacatggggattgccgttgtctgaagctaaagagcgaggtaagtgggtattagcatcagatttcccatttactagagaaactcttggtagttatgaaaagaaagctttttttgattctaataacgatgacatgttagttaaacttattattgacttcaaaaaaggtaacctcaaaaaagatatctctgatgcaaatttcatttatcgtaatgaaaatgtattagttgggtttgatgaactagttaattttattactgaagaacattga

SEQ ID NO: 7 - DNA, 144 bp, Escherichia coli
atgtgtggcattttcagtaaagaagtcctgagtaaacacgttgacgttgaataccgcttctctgccgagccttatattggtgcctcatgcagtaatgtgtcagttttatctatgttatgcctgcgggcgaagaaaacaatctaa

SEQ ID NO: 8 - DNA, 1221 bp, Escherichia coli
atgacaccaggcggacaagctcagataggtaatgttgatctcgtaaaacagcttaacagcgcggcggtttatcgcctgattgaccagtacgggccaatctcgcggattcagattgccgagcaaagccagcttgcccccgccagcgtaaccaaaattacgcgtcagcttatcgaacgcgggctgatcaaagaagttgatcagcaggcctccaccgggggccgccgcgctatctccatcgtcaccgaaacccgcaatttccacgcaatcggcgtacggcttggtcgtcatgacgccaccatcactctgtttgatctcagcagcaaagtgctggcagaagaacattacccgctgccggaacgtacccagcaaacgctggaacatgccctgttgaatgccattgctcagtttattgatagctaccagcgcaaactgcgcgagctgatcgcgatttcggtgatcctgccagggcttgttgacccggacagcggcaaaattcattacatgccgcatattcaggtagaaaactgggggctggtagaagctctggaagaacgttttaaagtgacctgtttcgttggtcacgatatccgtagtctggcgctggcggagcactacttcggtgcaagtcaggattgcgaagactccattctggtgcgtgtccatcgcggaaccggggccgggattatctctaacgggcgcatttttattggccgcaacggcaacgtcggtgaaattggccatattcaggtcgaaccgctgggtgaacgctgccactgcggcaactttggctgcctggaaactatcgctgccaacgctgccattgaacaacgggtgttgaatctgttaaagcagggctaccagagccgcgtgccgctggacgactgcaccatcaaaactatctgcaaagccgcgaacaaaggcgatagtctggcgtcggaagtaattgagtatgtcggtcgtcatctgggtaaaaccatcgccattgctatcaacttatttaatccgcaaaaaattgttattgccggtgaaatcaccgaagccgataaagtgctgctccctgctattgaaagctgcattaatacccaggcgctgaaggcgtttcgcactaatctgccggtggtacgttctgagctggatcaccgctcggcaatcggcgcttttgcgctggtaaaacgcgccatgctcaacggtattttgctccagcatttgctggaaaattaa

SEQ ID NO: 9 - DNA, 1149 bp, Escherichia coli
atgtatgcattaacccagggccggatctttaccggccacgaatttcttgatgaccacgcggttgttatcgctgatggcctgattaaaagcgtctgtccggtagcggaactgccgccagagatcgaacaacgttcactgaacggggccattctctcccccggttttatcgatgtgcagttaaacggctgcggcggcgtacagtttaacgacaccgctgaagcggtcagcgtggaaacgctggaaatcatgcagaaagccaatgagaaatcaggctgtactaactatctgccgacgcttatcaccaccagcgatgagctgatgaaacagggcgtgcgcgttatgcgcgagtacctggcaaaacatccgaatcaggcgttaggtctgcatctggaaggtccgtggctgaatctggtaaaaaaaggcacccataatccgaattttgtgcgtaagcctgatgccgcgctggtcgatttcctgtgtgaaaacgccgacgtcattaccaaagtgaccctggcaccggaaatggttcctgcggaagtcatcagcaaactggcaaatgccgggattgtggtttctgccggtcactccaacgcgacgttgaaagaagcaaaagccggtttccgcgcggggattacctttgccacccatctgtacaacgcgatgccgtatattaccggtcgtgaacctggcctggcgggcgcgatcctcgacgaagctgacatttattgcggtattattgctgatggcctgcatgttgattacgccaacattcgcaacgctaaacgtctgaaaggcgacaaactgtgtctggttactgacgccaccgcgccagcaggtgccaacattgaacagttcatttttgcgggtaaaacaatatactaccgtaacggactttgtgtggatgagaacggtacgttaagcggttcatccttaaccatgattgaaggcgtgcgtaatctggtcgaacattgcggtatcgcactggatgaagtgctacgtatggcgacgctctatccggcgcgtgcgattggcgttgagaaacgtctcggcacactcgccgcaggtaaagtagccaacctgactgcattcacacctgattttaaaatcaccaagaccatcgttaacggtaacgaggtcgtaactcaataa

SEQ ID NO: 10 - DNA, 717 bp, Escherichia coli
atgcgtccagcaggccgtagcaataatcaggtgcgtcccgttaccctgactcgtaactatacaaaacatgcagaaggctcggtgctggtcgaatttggcgataccaaagtgttgtgtaccgcctctattgaagaaggcgtgccgcgcttcctgaaaggtcagggccagggctggatcaccgcagagtacggcatgctgccacgttctacccacacccgtaacgctcgtgaagcggcgaaaggtaagcagggtggacgcacaatggaaatccagcgtctgatcgcccgtgctcttcgcgcggcagtagatttgaaagcgctgggtgagttcaccattacgctggactgcgacgtgcttcaggctgatggtggcacgcgtaccgcgtcgattacgggtgcctgcgtggcgctggtagatgcgctacagaagctggtggaaaacggcaagctgaaaaccaatccgatgaaagggatggtagccgcagtttctgtcggaattgtgaacggcgaagcggtttgcgatctggaatacgttgaagactctgccgcagagaccgacatgaacgtagtgatgaccgaagacgggcgcatcattgaagtgcaggggacggcagaaggcgagccgttcacccatgaagagctactcatcttgttggctctggcccgagggggaatcgaatccattgtagcgacgcagaaggcggcgctggcaaactga

SEQ ID NO: 11 - DNA, 879 bp, Escherichia coli
atgagcgacgacaattcacacagtagtgacacgataagcaacaagaagggatttttctccctgttactcagccaacttttccacggtgaaccgaaaaaccgtgacgaactgctggcgctgatccgtgattccgggcagaacgaccttatcgacgaagatacgcgcgatatgctcgaaggggtgatggacatcgcagaccaacgcgttcgcgacatcatgatcccccgctcccagatgattaccctgaaacgcaaccagacgctggacgaatgtcttgatgtcatcatcgagtccgcccactcacgtttcccggtgattagcgaagacaaagatcacattgaagggattctgatggcgaaagacttgctgccgtttatgcgcagcgatgctgaagccttcagcatggacaaagtgttacgtcaggcggttgtcgttcctgaaagtaagcgcgtagaccggatgctgaaagagtttcgctctcagcgttaccacatggcgatcgttattgacgaattcggtggggtttccggtctggtgaccattgaagacatcctggaactgattgttggtgagattgaagacgagtatgacgaagaagatgatatcgacttccgtcagctgagtcgtcatacctggaccgtacgcgcactggcttccattgaagacttcaacgaagcgttcggcacccactttagcgatgaagaagtcgacactatcggtggtctggtgatgcaggcatttgggcatcttccggcgcgtggcgaaactatcgacatcgacggttaccagttcaaagtggcgatggccgacagtcggcgtattattcaggttcatgtcaaaatcccggatgactcaccccagccgaagctggatgaataa

SEQ ID NO: 12 - DNA, 1374 bp, Escherichia coli
atgcgcattcatattttaggaatttgtggcacatttatgggcggtctggcgatgctggcgcgccagttaggccatgaagtaacgggttcggacgccaatgtgtatccgccgatgagcaccttacttgagaagcaaggcattgagctgattcaggggtacgatgccagccagctcgagccgcagccggatctggtgattattggcaacgccatgacccgtggaaatccgtgtgtggaagcggtactggaaaaaaatatcccttatatgtcaggtccacagtggctgcacgattttgtgctgcgcgaccgctgggtgctggccgttgccggtacacacggcaaaaccaccaccgcgggaatggcgacctggattctggaacagtgtggttacaaaccgggctttgtaatcggcggtgtgccggggaactttgaggtttcggctcatctgggcgaaagcgacttctttgttatcgaagcggatgagtatgactgcgccttcttcgacaaacgctctaaatttgttcattactgcccgcgtacgctgatcctcaacaaccttgagttcgatcacgccgatatctttgacgacctgaaagcgatccagaaacagttccaccatctggtgcgtatcgttccggggcagggccgtattatctggccggaaaatgacatcaacctgaaacagaccatggcgatgggctgctggagcgagcaggagctggtgggtgagcagggtcactggcaggcgaaaaagctgaccaccgatgcttccgaatgggaagttttgctggatggcgaaaaagtgggcgaagtgaaatggtcgctggtaggcgaacataatatgcacaatggcctgatggcgattgcagcggctcgccatgttggtgtagcgccggcagatgccgctaacgcgctgggttcgtttattaatgctcgtcgccgtctggagttgcgtggtgaagcgaatggcgtcacggtatatgacgattttgcccatcacccgacggcgattctggcaacgctggcggcgctgcgtggcaaagttggtggtacggcgcgcattattgctgtgctggagccgcgctcgaataccatgaaaatggggatctgcaaagacgatctggcaccttcattaggtcgtgccgatgaagtcttcctgctgcaaccggcgcatattccgtggcaggtggcagaagtggcagaagcctgcgttcagcctgcacactggagtggcgatgtggatacgctggcagatatggtggtgaaaaccgctcagcctggcgaccatattctggtgatgagcaacggcggttttggtgggatccatcagaaactgctggatgggctggcgaagaaggcggaagccgcgcagtaa

SEQ ID NO: 13 - DNA, 345 bp, Escherichia coli
atgtatttacgaccagacgaggtggcgcgcgtacttgaaaaagtcggttttactgtcgatgtggtaacccagaaagcgtatggttaccgccgtggggaaaattatgtctatgttaatcgcgaagcgcgaatgggacgtaccgctctggtaattcatccaacattaaaagaacgcagttcgacactggctgaacccgcttccgatattaaaacttgcgatcattaccagcaatttccgctctatttagcaggcgagcgacacgagcattacggtatcccgcatggctttagttcgcgtgttgcgcttgaacgttatttgaatggtttatttggcgaagccagttaa

SEQ ID NO: 14 - protein, 114 aa, Escherichia coli
MYLRPDEVARVLEKVGFTVDVVTQKAYGYRRGENYVYVNREARMGRTALVIHPTLKERSSTLAEPASDIKTCDHYQQFPLYLAGERHEHYGIPHGFSSRVALERYLNGLFGEAS

SEQ ID NO: 15 - DNA, 540 bp, Escherichia coli
atgccacgtcgtcgcgtcattggtcagcgtaaaattctgccggatccgaagttcggatcagaactgctggctaaatttgtaaatatcctgatggtagatggtaaaaaatctactgctgaatctatcgtatacagcgcgctggagaccctggctcagcgctctggtaaatctgaactggaagcattcgaagtagctctcgaaaacgtgcgcccgactgtagaagttaagtctcgccgcgttggtggttctacttatcaggtaccagttgaagtccgtccggttcgtcgtaatgctctggcaatgcgttggatcgttgaagctgctcgtaaacgcggtgataaatccatggctctgcgcctggcgaacgaactttctgatgctgcagaaaacaaaggtactgcagttaagaaacgtgaagacgttcaccgtatggccgaagccaacaaggcgttcgcacactaccgttggttatcccttcggagttttagtcaccaggcgggcgcttccagtaagcagcccgctttgggctacttaaattga

SEQ ID NO: 16 - protein, 179 aa, Escherichia coli
MPRRRVIGQRKILPDPKFGSELLAKFVNILMVDGKKSTAESIVYSALETLAQRSGKSELEAFEVALENVRPTVEVKSRRVGGSTYQVPVEVRPVRRNALAMRWIVEAARKRGDKSMALRLANELSDAAENKGTAVKKREDVHRMAEANKAFAHYRWLSLRSFSHQAGASSKQPALGYLN

SEQ ID NO: 17 - DNA, 1344 bp, Escherichia coli
atgacgacgattctcaagcatctcccggtaggtcaacgtattggtatcgctttttctggcggtctggacaccagtgccgcactgctgtggatgcgacaaaagggagcggttccttatgcatatactgcaaacctgggccagccagacgaagaggattatgatgcgatccctcgtcgtgccatggaatacggcgcggagaacgcacgtctgatcgactgccgcaaacaactggtggccgaaggtattgccgctattcagtgtggcgcatttcataacaccaccggcggcctgacctatttcaacacgacgccgctgggccgcgccgtgactggtaccatgctggttgctgcgatgaaagaagatggcgtgaatatctggggtgacggtagcacctacaaaggaaacgatatcgaacgtttctatcgttatggtctgctgaccaatgctgaactgcagatttacaaaccgtggcttgatactgactttattgatgaactgggcggccgtcatgagatgtctgaatttatgattgcctgcggtttcgactacaaaatgtctgtcgaaaaagcctactccacagactccaacatgcttggtgcaacgcatgaagcgaaggatctggaatacctcaactccagcgtcaaaatcgtcaacccgattatgggcgtgaaattctgggatgagagcgtgaagatcccggcagaagaagtcacagtacgctttgaacaaggtcatccggtggcgctgaacggtaaaacctttagcgacgacgtagaaatgatgctggaagctaaccgcatcggcggtcgtcacggcctgggcatgagcgaccagattgaaaaccgtatcatcgaagcgaaaagccgtggtatttacgaagctccggggatggcactgctgcacattgcgtatgaacgcctgttgaccggtattcacaacgaagacaccattgagcagtatcacgcgcatggtcgtcagttgggccgtctgctgtaccaggggcgttggtttgactcccaggcgctgatgctgcgtgactctctgcaacgctgggttgccagccagatcactggtgaagttaccctggagctgcgccgtgggaacgattattcaatcctgaataccgtctcagagaacctgacctacaagccagagcgtctgacgatggaaaaaggcgactcggtgttctcgccagatgatcgtattggtcaattgaccatgcgtaacctggatatcactgatacccgcgagaaacttttcggttatgccaaaactggcctgctttcctcctctgccgcttcaggcgtgccgcaggtggagaatctggaaaacaaaggccagtaa

SEQ ID NO: 18 - protein, 447 aa, Escherichia coli
MTTILKHLPVGQRIGIAFSGGLDTSAALLWMRQKGAVPYAYTANLGQPDEEDYDAIPRRAMEYGAENARLIDCRKQLVAEGIAAIQCGAFHNTTGGLTYFNTTPLGRAVTGTMLVAAMKEDGVNIWGDGSTYKGNDIERFYRYGLLTNAELQIYKPWLDTDFIDELGGRHEMSEFMIACGFDYKMSVEKAYSTDSNMLGATHEAKDLEYLNSSVKIVNPIMGVKFWDESVKIPAEEVTVRFEQGHPVALNGKTFSDDVEMMLEANRIGGRHGLGMSDQIENRIIEAKSRGIYEAPGMALLHIAYERLLTGIHNEDTIEQYHAHGRQLGRLLYQGRWFDSQALMLRDSLQRWVASQITGEVTLELRRGNDYSILNTVSENLTYKPERLTMEKGDSVFSPDDRIGQLTMRNLDITDTREKLFGYAKTGLLSSSAASGVPQVENLENKGQ

SEQ ID NO: 19 - DNA, 1044 bp, Escherichia coli
atgttgaaaaaatttcgtggcatgttttccaatgacttgtccattgacctgggtactgcgaataccctcatttatgtaaaaggacaaggcatcgtattgaatgagccttccgtggtggccattcgtcaggatcgtgccggttcaccgaaaagcgtagctgcagtaggtcatgacgcgaagcagatgctgggccgtacgccgggcaatattgctgccattcgcccaatgaaagacggcgttatcgccgacttcttcgtgactgaaaaaatgctccagcacttcatcaaacaagtgcacagcaacagctttatgcgtccaagcccgcgcgttctggtttgtgtgccggttggcgcgacccaggttgaacgccgcgcaattcgtgaatccgcgcagggcgctggtgcccgtgaagtcttcctgattgaagaaccgatggctgccgcaattggtgctggcctgccggtttctgaagcgaccggttctatggtggttgatatcggtggtggtaccactgaagttgctgttatctccttgaacggtgtggtttactcctcttctgtgcgcattggtggtgaccgtttcgacgaagctatcatcaactatgtgcgtcgtaattacggttctctgatcggtgaagccaccgcagaacgtatcaagcacgaaatcggttcggcttatccgggcgatgaagtccgtgaaatcgaagttcgtggccgtaacctggcagaaggtgttccacgcggttttaccctgaactccaatgaaatcctcgaagcactgcaggaaccgctgaccggtattgtgagcgcggtaatggttgcactggaacagtgcccgccggaactggcttccgacatctccgagcgcggcatggtgctcaccggtggtggcgcactgctgcgtaaccttgaccgtttgttaatggaagaaaccggcattccagtcgttgttgctgaagacccgctgacctgtgtggcgcgcggtggcggcaaagcgctggaaatgatcgacatgcacggcggcgacctgttcagcgaagagtaa

SEQ ID NO: 20 - protein, 347 aa, Escherichia coli
MLKKFRGMFSNDLSIDLGTANTLIYVKGQGIVLNEPSVVAIRQDRAGSPKSVAAVGHDAKQMLGRTPGNIAAIRPMKDGVIADFFVTEKMLQHFIKQVHSNSFMRPSPRVLVCVPVGATQVERRAIRESAQGAGAREVFLIEEPMAAAIGAGLPVSEATGSMVVDIGGGTTEVAVISLNGVVYSSSVRIGGDRFDEAIINYVRRNYGSLIGEATAERIKHEIGSAYPGDEVREIEVRGRNLAEGVPRGFTLNSNEILEALQEPLTGIVSAVMVALEQCPPELASDISERGMVLTGGGALLRNLDRLLMEETGIPVVVAEDPLTCVARGGGKALEMIDMHGGDLFSEE

SEQ ID NO: 21 - DNA, 639 bp, Escherichia coli
atggctgtcgctgccaacaaacgttcggtaatgacgctgttttccggtcctactgacatctatagccatcaggtccgcattgtgctggctgagaaaggtgtaagtttcgagatcgaacacgtggaaaaggacaatccgcctcaggatctgattgacctcaacccgaatcagagcgttccgaccctggtggatcgtgagctgaccctgtgggaatctcgcatcattatggaatatctggatgagcgtttcccgcatccgccactgatgcctgtttacccggtagctcgcggtgaaagccgtctgtacatgcatcgcatcgaaaaagactggtacacgctgatgaacaccatcatcaacggttcagcttctgaagcagatgccgcacgtaagcaactgcgcgaagaactgctggcgattgcgccggtcttcggtcagaagccgtacttcctgagcgatgagttcagcctggtcgattgctatcttgctccgctgctgtggcgtctgccgcaactgggcatcgagttcagcggcccgggtgcgaaagagctgaaaggctatatgacccgcgtctttgagcgtgactctttccttgcttctttaactgaagcagaacgtgaaatgcgtctgggccggagttaa

SEQ ID NO: 22 - protein, 212 aa, Escherichia coli
MAVAANKRSVMTLFSGPTDIYSHQVRIVLAEKGVSFEIEHVEKDNPPQDLIDLNPNQSVPTLVDRELTLWESRIIMEYLDERFPHPPLMPVYPVARGESRLYMHRIEKDWYTLMNTIINGSASEADAARKQLREELLAIAPVFGQKPYFLSDEFSLVDCYLAPLLWRLPQLGIEFSGPGAKELKGYMTRVFERDSFLASLTEAEREMRLGRS

SEQ ID NO: 23 - DNA, 1488 bp, Escherichia coli
atgaacaaagaaattttggctgtagttgaagccgtatccaatgaaaaggcgctacctcgcgagaagattttcgaagcattggaaagcgcgctggcgacagcaacaaagaaaaaatatgaacaagagatcgacgtccgcgtacagatcgatcgcaaaagcggtgattttgacactttccgtcgctggttagttgttgatgaagtcacccagccgaccaaggaaatcacccttgaagccgcacgttatgaagatgaaagcctgaacctgggcgattacgttgaagatcagattgagtctgttacctttgaccgtatcactacccagacggcaaaacaggttatcgtgcagaaagtgcgtgaagccgaacgtgcgatggtggttgatcagttccgtgaacacgaaggtgaaatcatcaccggcgtggtgaaaaaagtaaaccgcgacaacatctctctggatctgggcaacaacgctgaagccgtgatcctgcgcgaagatatgctgccgcgtgaaaacttccgccctggcgaccgcgttcgtggcgtgctctattccgttcgcccggaagcgcgtggcgcgcaactgttcgtcactcgttccaagccggaaatgctgatcgaactgttccgtattgaagtgccagaaatcggcgaagaagtgattgaaattaaagcagcggctcgcgatccgggttctcgtgcgaaaatcgcggtgaaaaccaacgataaacgtatcgatccggtaggtgcttgcgtaggtatgcgtggcgcgcgtgttcaggcggtgtctactgaactgggtggcgagcgtatcgatatcgtcctgtgggatgataacccggcgcagttcgtgattaacgcaatggcaccggcagacgttgcttctatcgtggtggatgaagataaacacaccatggatatcgccgttgaagccggtaacctggcgcaggcgattggccgtaacggtcagaacgtgcgtctggcttcgcagctgagcggttgggaactcaacgtgatgaccgttgacgacctgcaggctaagcatcaggcggaagcgcacgcagcgatcgacaccttcaccaaatatctcgacatcgacgaagacttcgcgactgttctggtagaagaaggcttctcgacgctggaagaattggcctatgtgccgatgaaagagctgttggaaatcgaaggccttgatgagccgaccgttgaagcactgcgcgagcgtgctaaaaatgcactggccaccattgcacaggcccaggaagaaagcctcggtgataacaaaccggctgacgatctgctgaaccttgaaggggtagatcgtgatttggcattcaaactggccgcccgtggcgtttgtacgctggaagatctcgccgaacagggcattgatgatctggctgatatcgaagggttgaccgacgaaaaagccggagcactgattatggctgcccgtaatatttgctggttcggtgacgaagcgtaa

SEQ ID NO: 24 - protein, 495 aa, Escherichia coli [sequence omitted]

SEQ ID NO: 25 - DNA, 1113 bp, Escherichia coli
atgacggataatccgaataaaaaaacattctgggataaagtccatctcgatcccacaatgctgctgatcttactggcattgctggtttacagcgccctggttatctggagcgccagcggtcaggatattggcatgatggagcgtaaaatcggccaaatcgcgatgggtctggtcatcatggtggtgatggcgcaaattcctccacgcgtttatgaaggctgggccccctatctctatatcatctgtattattttgctggtggcggtagatgctttcggtgccatctctaaaggtgctcaacgctggctggacctcggtattgttcgttttcagccgtcggaaattgccaaaatagccgtaccactgatggttgcgcgctttatcaaccgcgacgtttgcccgccatcgttgaagaacactggcatcgcgctggtgctgatatttatgcccacgctgctggtggctgcacagcctgacctgggaacatcaatcctcgttgcgctttccggtctgtttgtactgttcctctctggccttagctggcgtctgattggcgtcgcagtagtgctggtagcggcgttcattccgattctgtggttcttcctgatgcatgattaccagcgccagcgcgtaatgatgctcctggacccggaatcagacccactcggcgcgggctatcacattattcagtctaaaattgctattggctccggcggattacgcggcaaaggctggctgcacggcactcagtcacagcttgaatttctccccgaacgccatactgactttatcttcgcggtactggcggaagagctgggattagtgggcattctgattctgctcgctctctacattctgctgatcatgcgcgggctgtggatagccgccagagcgcaaaccacctttggtcgcgtcatggctggcggcttaatgctgatattattcgtttatgtcttcgtaaatattggtatggtaagcggtattctgccggttgtaggggttccgctcccactggtcagttatggaggatcggcgctaattgtgctgatggctgggttcgggattgtaatgtcaatccacacccacaggaaaatgttgtcgaaaagcgtgtaa

SEQ ID NO: 26 - protein, 370 aa, Escherichia coli
MTDNPNKKTFWDKVHLDPTMLLILLALLVYSALVIWSASGQDIGMMERKIGQIAMGLVIMVVMAQIPPRVYEGWAPYLYIICIILLVAVDAFGAISKGAQRWLDLGIVRFQPSEIAKIAVPLMVARFINRDVCPPSLKNTGIALVLIFMPTLLVAAQPDLGTSILVALSGLFVLFLSGLSWRLIGVAVVLVAAFIPILWFFLMHDYQRQRVMMLLDPESDPLGAGYHIIQSKIAIGSGGLRGKGWLHGTQSQLEFLPERHTDFIFAVLAEELGLVGILILLALYILLIMRGLWIAARAQTTFGRVMAGGLMLILFVYVFVNIGMVSGILPVVGVPLPLVSYGGSALIVLMAGFGIVMSIHTHRKMLSKSV

SEQ ID NO: 27 - DNA, 1842 bp, Escherichia coli
atggagcaaaacccgcagtcacagctgaaacttcttgtcacccgtggtaaggagcaaggctatctgacctatgccgaggtcaatgaccatctgccggaagatatcgtcgattcagatcagatcgaagacatcatccaaatgatcaacgacatgggcattcaggtgatggaagaagcaccggatgccgatgatctgatgctggctgaaaacaccgcggacgaagatgctgccgaagccgccgcgcaggtgctttccagcgtggaatctgaaatcgggcgcacgactgacccggtacgcatgtacatgcgtgaaatgggcaccgttgaactgttgacccgcgaaggcgaaattgacatcgctaagcgtattgaagacgggatcaaccaggttcaatgctccgttgctgaatatccggaagcgatcacctatctgctggaacagtacgatcgtgttgaagcagaagaagcgcgtctgtccgatctgatcaccggctttgttgacccgaacgcagaagaagatctggcacctaccgccactcacgtcggttctgagctttcccaggaagatctggacgatgacgaagatgaagacgaagaagatggcgatgacgacagcgccgatgatgacaacagcatcgacccggaactggctcgcgaaaaatttgcggaactacgcgctcagtacgttgtaacgcgtgacaccatcaaagcgaaaggtcgcagtcacgctaccgctcaggaagagatcctgaaactgtctgaagtattcaaacagttccgcctggtgccgaagcagtttgactacctggtcaacagcatgcgcgtcatgatggaccgcgttcgtacgcaagaacgtctgatcatgaagctctgcgttgagcagtgcaaaatgccgaagaaaaacttcattaccctgtttaccggcaacgaaaccagcgatacctggttcaacgcggcaattgcgatgaacaagccgtggtcggaaaaactgcacgatgtctctgaagaagtgcatcgcgccctgcaaaaactgcagcagattgaagaagaaaccggcctgaccatcgagcaggttaaagatatcaaccgtcgtatgtccatcggtgaagcgaaagcccgccgtgcgaagaaagagatggttgaagcgaacttacgtctggttatttctatcgctaagaaatacaccaaccgtggcttgcagttccttgacctgattcaggaaggcaacatcggtctgatgaaagcggttgataaattcgaataccgccgtggttacaagttctccacctacgcaacctggtggatccgtcaggcgatcacccgctctatcgcggatcaggcgcgcaccatccgtattccggtgcatatgattgagaccatcaacaagctcaaccgtatttctcgccagatgctgcaagagatgggccgtgaaccgacgccggaagaactggctgaacgtatgctgatgccggaagacaagatccgcaaagtgctgaagatcgccaaagagccaatctccatggaaacgccgatcggtgatgatgaagattcgcatctgggggatttcatcgaggataccaccctcgagctgccgctggattctgcgaccaccgaaagcctgcgtgcggcaacgcacgacgtgctggctggcctgaccgcgcgtgaagcaaaagttctgcgtatgcgtttcggtatcgatatgaacaccgactacacgctggaagaagtgggtaaacagttcgacgttacccgcgaacgtatccgtcagatcgaagcgaaggcgctgcgcaaactgcgtcacccgagccgttctgaagtgctgcgtagcttcctggacgattaa

SEQ ID NO: 28 - protein, 613 aa, Escherichia coli
MEQNPQSQLKLLVTRGKEQGYLTYAEVNDHLPEDIVDSDQIEDIIQMINDMGIQVMEEAPDADDLMLAENTADEDAAEAAAQVLSSVESEIGRTTDPVRMYMREMGTVELLTREGEIDIAKRIEDGINQVQCSVAEYPEAITYLLEQYDRVEAEEARLSDLITGFVDPNAEEDLAPTATHVGSELSQEDLDDDEDEDEEDGDDDSADDDNSIDPELAREKFAELRAQYVVTRDTIKAKGRSHATAQEEILKLSEVFKQFRLVPKQFDYLVNSMRVMMDRVRTQERLIMKLCVEQCKMPKKNFITLFTGNETSDTWFNAAIAMNKPWSEKLHDVSEEVHRALQKLQQIEEETGLTIEQVKDINRRMSIGEAKARRAKKEMVEANLRLVISIAKKYTNRGLQFLDLIQEGNIGLMKAVDKFEYRRGYKFSTYATWWIRQAITRSIADQARTIRIPVHMIETINKLNRISRQMLQEMGREPTPEELAERMLMPEDKIRKVLKIAKEPISMETPIGDDEDSHLGDFIEDTTLELPLDSATTESLRAATHDVLAGLTAREAKVLRMRFGIDMNTDYTLEEVGKQFDVTRERIRQIEAKALRKLRHPSRSEVLRSFLDD

SEQ ID NO: 29 - DNA, 4224 bp, Escherichia coli
gtgaaagatttattaaagtttctgaaagcgcagactaaaaccgaagagtttgatgcgatcaaaattgctctggcttcgccagacatgatccgttcatggtctttcggtgaagttaaaaagccggaaaccatcaactaccgtacgttcaaaccagaacgtgacggccttttctgcgcccgtatctttgggccggtaaaagattacgagtgcctgtgcggtaagtacaagcgcctgaaacaccgtggcgtcatctgtgagaagtgcggcgttgaagtgacccagactaaagtacgccgtgagcgtatgggccacatcgaactggcttccccgactgcgcacatctggttcctgaaatcgctgccgtcccgtatcggtctgctgctcgatatgccgctgcgcgatatcgaacgcgtactgtactttgaatcctatgtggttatcgaaggcggtatgaccaacctggaacgtcagcagatcctgactgaagagcagtatctggacgcgctggaagagttcggtgacgaattcgacgcgaagatgggggcggaagcaatccaggctctgctgaagagcatggatctggagcaagagtgcgaacagctgcgtgaagagctgaacgaaaccaactccgaaaccaagcgtaaaaagctgaccaagcgtatcaaactgctggaagcgttcgttcagtctggtaacaaaccagagtggatgatcctgaccgttctgccggtactgccgccagatctgcgtccgctggttccgctggatggtggtcgtttcgcgacttctgacctgaacgatctgtatcgtcgcgtcattaaccgtaacaaccgtctgaaacgtctgctggatctggctgcgccggacatcatcgtacgtaacgaaaaacgtatgctgcaggaagcggtagacgccctgctggataacggtcgtcgcggtcgtgcgatcaccggttctaacaagcgtcctctgaaatctttggccgacatgatcaaaggtaaacagggtcgtttccgtcagaacctgctcggtaagcgtgttgactactccggtcgttctgtaatcaccgtaggtccatacctgcgtctgcatcagtgcggtctgccgaagaaaatggcactggagctgttcaaaccgttcatctacggcaagctggaactgcgtggtcttgctaccaccattaaagctgcgaagaaaatggttgagcgcgaagaagctgtcgtttgggatatcctggacgaagttatccgcgaacacccggtactgctgaaccgtgcaccgactctgcaccgtctgggtatccaggcatttgaaccggtactgatcgaaggtaaagctatccagctgcacccgctggtttgtgcggcatataacgccgacttcgatggtgaccagatggctgttcacgtaccgctgacgctggaagcccagctggaagcgcgtgcgctgatgatgtctaccaacaacatcctgtccccggcgaacggcgaaccaatcatcgttccgtctcaggacgttgtactgggtctgtactacatgacccgtgactgtgttaacgccaaaggcgaaggcatggtgctgactggcccgaaagaagcagaacgtctgtatcgctctggtctggcttctctgcatgcgcgcgttaaagtgcgtatcaccgagtatgaaaaagatgctaacggtgaattagtagcgaaaaccagcctgaaagacacgactgttggccgtgccattctgtggatgattgtaccgaaaggtctgccttactccatcgtcaaccaggcgctgggtaaaaaagcaatctccaaaatgctgaacacctgctaccgcattctcggtctgaaaccgaccgttatttttgcggaccagatcatgtacaccggcttcgcctatgcagcgcgttctggtgcatctgttggtatcgatgacatggtcatcccggagaagaaacacgaaatcatctccgaggcagaagcagaagttgctgaaattcaggagcagttccagtctggtctggtaactgcgggcgaacgctacaacaaagttatcgatatctgggctgcggcgaacgatcgtgtatccaaagcgatgatggataacctgcaaactgaaaccgtgattaaccgtgacggtcaggaagagaagcaggtttccttcaacagcatctacatgatggccgactccggtgcgcgtggttctgcggcacagattcgtcagcttgctggtatgcgtggtctgatggcgaagccggatggctccatcatcgaaacgccaatcaccgcgaacttccgtgaaggtctgaacgtactccagtacttcatctccacccacggtgctcgtaaaggtctggcggataccgcactgaaaactgcgaactccggttacctgactcgtcgtctggttgacgtggcgcaggacctggtggttaccgaagacgattgtggtacccatgaaggtatcatgatgactccggttatcgagggtggtgacgttaaagagccgctgcgcgatcgcgtactgggtcgtgtaactgctgaagacgttctgaagccgggtactgctgatatcctcgttccgcgcaacacgctgctgcacgaacagtggtgtgacctgctggaagagaactctgtcgacgcggttaaagtacgttctgttgtatcttgtgacaccgactttggtgtatgtgcgcactgctacggtcgtgacctggcgcgtggccacatcatcaacaagggtgaagcaatcggtgttatcgcggcacagtccatcggtgaaccgggtacacagctgaccatgcgtacgttccacatcggtggtgcggcatctcgtgcggctgctgaatccagcatccaagtgaaaaacaaaggtagcatcaagctcagcaacgtgaagtcggttgtgaactccagcggtaaactggttatcacttcccgtaatactgaactgaaactgatcgacgaattcggtcgtactaaagaaagctacaaagtaccttacggtgcggtactggcgaaaggcgatggcgaacaggttgctggcggcgaaaccgttgcaaactgggacccgcacaccatgccggttatcaccgaagtaagcggttttgtacgctttactgacatgatcgacggccagaccattacgcgtcagaccgacgaactgaccggtctgtcttcgctggtggttctggattccgcagaacgtaccgcaggtggtaaagatctgcgtccggcactgaaaatcgttgatgctcagggtaacgacgttctgatcccaggtaccgatatgccagcgcagtacttcctgccgggtaaagcgattgttcagctggaagatggcgtacagatcagctctggtgacaccctggcgcgtattccgcaggaatccggcggtaccaaggacatcaccggtggtctgccgcgcgttgcggacctgttcgaagcacgtcgtccgaaagagccggcaatcctggctgaaatcagcggtatcgtttccttcggtaaagaaaccaaaggtaaacgtcgtctggttatcaccccggtagacggtagcgatccgtacgaagagatgattccgaaatggcgtcagctcaacgtgttcgaaggtgaacgtgtagaacgtggtgacgtaatttccgacggtccggaagcgccgcacgacattctgcgtctgcgtggtgttcatgctgttactcgttacatcgttaacgaagtacaggacgtataccgtctgcagggcgttaagattaacgataaacacatcgaagttatcgttcgtcagatgctgcgtaaagctaccatcgttaacgcgggtagctccgacttcctggaaggcgaacaggttgaatactctcgcgtcaagatcgcaaaccgcgaactggaagcgaacggcaaagtgggtgcaacttactcccgcgatctgctgggtatcaccaaagcgtctctggcaaccgagtccttcatctccgcggcatcgttccaggagaccactcgcgtgctgaccgaagcagccgttgcgggcaaacgcgacgaactgcgcggcctgaaagagaacgttatcgtgggtcgtctgatcccggcaggtaccggttacgcgtaccaccaggatcgtatgcgtcgccgtgctgcgggtgaagctccggctgcaccgcaggtgactgcagaagacgcatctgccagcctggcagaactgctgaacgcaggtctgggcggttctgataacgagtaa

SEQ ID NO: 30 - protein, 1407 aa, Escherichia coli [sequence omitted]

SEQ ID NO: 31 - DNA, 4029 bp, Escherichia coli
atggtttactcctataccgagaaaaaacgtattcgtaaggattttggtaaacgtccacaagttctggatgtaccttatctcctttctatccagcttgactcgtttcagaaatttatcgagcaagatcctgaagggcagtatggtctggaagctgctttccgttccgtattcccgattcagagctacagcggtaattccgagctgcaatacgtcagctaccgccttggcgaaccggtgtttgacgtccaggaatgtcaaatccgtggcgtgacctattccgcaccgctgcgcgttaaactgcgtctggtgatctatgagcgcgaagcgccggaaggcaccgtaaaagacattaaagaacaagaagtctacatgggcgaaattccgctcatgacagacaacggtacctttgttatcaacggtactgagcgtgttatcgtttcccagctgcaccgtagtccgggcgtcttctttgactccgacaaaggtaaaacccactcttcgggtaaagtgctgtataacgcgcgtatcatcccttaccgtggttcctggctggacttcgaattcgatccgaaggacaacctgttcgtacgtatcgaccgtcgccgtaaactgcctgcgaccatcattctgcgcgccctgaactacaccacagagcagatcctcgacctgttctttgaaaaagttatctttgaaatccgtgataacaagctgcagatggaactggtgccggaacgcctgcgtggtgaaaccgcatcttttgacatcgaagctaacggtaaagtgtacgtagaaaaaggccgccgtatcactgcgcgccacattcgccagctggaaaaagacgacgtcaaactgatcgaagtcccggttgagtacatcgcaggtaaagtggttgctaaagactatattgatgagtctaccggcgagctgatctgcgcagcgaacatggagctgagcctggatctgctggctaagctgagccagtctggtcacaagcgtatcgaaacgctgttcaccaacgatctggatcacggcccatatatctctgaaaccttacgtgtcgacccaactaacgaccgtctgagcgcactggtagaaatctaccgcatgatgcgccctggcgagccgccgactcgtgaagcagctgaaagcctgttcgagaacctgttcttctccgaagaccgttatgacttgtctgcggttggtcgtatgaagttcaaccgttctctgctgcgcgaagaaatcgaaggttccggtatcctgagcaaagacgacatcattgatgttatgaaaaagctcatcgatatccgtaacggtaaaggcgaagtcgatgatatcgaccacctcggcaaccgtcgtatccgttccgttggcgaaatggcggaaaaccagttccgcgttggcctggtacgtgtagagcgtgcggtgaaagagcgtctgtctctgggcgatctggataccctgatgccacaggatatgatcaacgccaagccgatttccgcagcagtgaaagagttcttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggcttccttgagactccgtatcgtaaagtgaccgacggtgttgtaactgacgaaattcactacctgtctgctatcgaagaaggcaactacgttatcgcccaggcgaactccaacttggatgaagaaggccacttcgtagaagacctggtaacttgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccgactctgcgcgctgataagccgctggttggtactggtatggaacgtgctgttgccgttgactccggtgtaactgcggtagctaaacgtggtggtgtcgttcagtacgtggatgcttcccgtatcgttatcaaagttaacgaagacgagatgtatccgggtgaagcaggtatcgacatctacaacctgaccaaatacacccgttctaaccagaacacctgtatcaaccagatgccgtgtgtgtctctgggtgaaccggttgaacgtggcgacgtgctggcagacggtccgtccaccgacctcggtgaactggcgcttggtcagaacatgcgcgtagcgttcatgccgtggaatggttacaacttcgaagactccatcctcgtatccgagcgtgttgttcaggaagaccgtttcaccaccatccacattcaggaactggcgtgtgtgtcccgtgacaccaagctgggtccggaagagatcaccgctgacatcccgaacgtgggtgaagctgcgctctccaaactggatgaatccggtatcgtttacattggtgcggaagtgaccggtggcgacattctggttggtaaggtaacgccgaaaggtgaaactcagctgaccccagaagaaaaactgctgcgtgcgatcttcggtgagaaagcctctgacgttaaagactcttctctgcgcgtaccaaacggtgtatccggtacggttatcgacgttcaggtctttactcgcgatggcgtagaaaaagacaaacgtgcgctggaaatcgaagaaatgcagctcaaacaggcgaagaaagacctgtctgaagaactgcagatcctcgaagcgggtctgttcagccgtatccgtgctgtgctggtagccggtggcgttgaagctgagaagctcgacaaactgccgcgcgatcgctggctggagctgggcctgacagacgaagagaaacaaaatcagctggaacagctggctgagcagtatgacgaactgaaacacgagttcgagaagaaactcgaagcgaaacgccgcaaaatcacccagggcgacgatctggcaccgggcgtgctgaagattgttaaggtatatctggcggttaaacgccgtatccagcctggtgacaagatggcaggtcgtcacggtaacaagggtgtaatttctaagatcaacccgatcgaagatatgccttacgatgaaaacggtacgccggtagacatcgtactgaacccgctgggcgtaccgtctcgtatgaacatcggtcagatcctcgaaacccacctgggtatggctgcgaaaggtatcggcgacaagatcaacgccatgctgaaacagcagcaagaagtcgcgaaactgcgcgaattcatccagcgtgcgtacgatctgggcgctgacgttcgtcagaaagttgacctgagtaccttcagcgatgaagaagttatgcgtctggctgaaaacctgcgcaaaggtatgccaatcgcaacgccggtgttcgacggtgcgaaagaagcagaaattaaagagctgctgaaacttggcgacctgccgacttccggtcagatccgcctgtacgatggtcgcactggtgaacagttcgagcgtccggtaaccgttggttacatgtacatgctgaaactgaaccacctggtcgacgacaagatgcacgcgcgttccaccggttcttacagcctggttactcagcagccgctgggtggtaaggcacagttcggtggtcagcgtttcggggagatggaagtgtgggcgctggaagcatacggcgcagcatacaccctgcaggaaatgctcaccgttaagtctgatgacgtgaacggtcgtaccaagatgtataaaaacatcgtggacggcaaccatcagatggagccgggcatgccagaatccttcaacgtattgttgaaagagattcgttcgctgggtatcaacatcgaactggaagacgagtaa

SEQ ID NO: 32 - protein, 1342 aa, Escherichia coli [sequence omitted]

SEQ ID NO: 33 - DNA, 1260 bp, Escherichia coli
atggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataa

SEQ ID NO: 34 - protein, 419 aa, Escherichia coli
MDKFRVQGPTKLQGEVTISGAKNAALPILFAALLAEEPVEIQNVPKLKDVDTSMKLLSQLGAKVERNGSVHIDARDVNVFCAPYDLVKTMRASIWALGPLVARFGQGQVSLPGGCTIGARPVDLHISGLEQLGATIKLEEGYVKASVDGRLKGAHIVMDKVSVGATVTIMCAATLAEGTTIIENAAREPEIVDTANFLITLGAKISGQGTDRIVIEGVERLGGGVYRVLPDRIETGTFLVAAAISRGKIICRNAQPDTLDAVLAKLRDAGADIEVGEDWISLDMHGKRPKAVNVRTAPHPAFPTDMQAQFTLLNLVAEGTGFITETVFENRFMHVPELSRMGAHAEIESNTVICHGVEKLSGAQVMATDLRASASLVLAGCIAEGTTVVDRIYHIDRGYERIEDKLRALGANIERVKGE

SEQ ID NO: 35 - DNA, 1674 bp, Escherichia coli
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggactggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaa

SEQ ID NO: 36 - protein, 557 aa, Escherichia coli
MTESFAQLFEESLKEIETRPGSIVRGVVVAIDKDVVLVDAGLKSESAIPAEQFKNAQGELEIQVGDEVDVALDAVEDGFGETLLSREKAKRHEAWITLEKAYEDAETVTGVINGKVKGGFTVELNGIRAFLPGSLVDVRPVRDTLHLEGKELEFKVIKLDQKRNNVVVSRRAVIESENSAERDQLLENLQEGMEVKGIVKNLTDYGAFVDLGGVDGLLHITDMAWKRVKHPSEIVNVGDEITVKVLKFDRERTRVSLGLKQLGEDPWVAIAKRYPEGTKLTGRVTNLTDYGCFVEIEEGVEGLVHVSEMDWTNKNIHPSKVVNVGDVVEVMVLDIDEERRRISLGLKQCKANPWQQFAETHNKGDRVEGKIKSITDFGIFIGLDGGIDGLVHLSDISWNVAGEEAVREYKKGDEIAAVVLQVDAERERISLGVKQLAEDPFNNWVALNKKGAIVTGKVTAVDAKGATVELADGVEGYLRASEASRDRVEDATLVLSVGDEVEAKFTGVDRKNRAISLSVRAKDEADEKDAIATVNKQEDANFSNNAMAEAFKAAKGE

SEQ ID NO: 37 - DNA, 2109 bp, Escherichia coli
ttgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttatagcccgctggcgcaccgtttaggtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgagaagcatctttattcgatttactgcaaaatggtgctcaaagagcagcgttttcactcgatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgatcggcccgcacggtgtgccggttgaggtccagatccgtaccgaagatatggaccagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggtcatgcctgcgtgggcgcacgcgttgaccgccagccttacccgctgtcgcagccgcttaccagcggtcaaaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctgaatgaaatcccgcaggaaaatattcagcgcgagctggatcgcatgaagctggcaacgcttgacgatctgctggcagaaatcggacttggtaacgcaatgagcgtggtggtcgcgaaaaatctgcaacatggggacgcctccattccaccggcaacccaaagccacggacatctgcccattaaaggtgccgatggcgtgctgatcacctttgcgaaatgctgccgccctattcctggcgacccgattatcgcccacgtcagccccggtaaaggtctggtgatccaccatgaatcctgccgtaatatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacttcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 38 - protein, 702 aa, Escherichia coli
MYLFESLNQLIQTYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLGIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN

SEQ ID NO: 39 - DNA, 642 bp, Escherichia coli
atgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaa

SEQ ID NO: 40 - protein, 213 aa, Escherichia coli
MKPYQRQFIEFALSKQVLKFGEFTLKSGRKSPYFFNAGLFNTGRDLALLGRFYAEALVDSGIEFDLLFGPAYKGIPIATTTAVALAEHHDLDLPYCFNRKEAKDHGEGGNLVGSALQGRVMLVDDVITAGTAIRESMEIIQANGATLAGVLISLDRQERGRGEISAIQEVERDYNCKVISIITLKDLIAYLEEKPEMAEHLAAVKAYREEFGV

SEQ ID NO: 41 - DNA, 65 bp, Escherichia coli
gccttcgctcctcatcttacttttctacagacaaaaaaaaggcgactcatcagtcgccttaaaaa

SEQ ID NO: 42 - DNA, 864 bp, Escherichia coli
atgatccgcagtatgaccgcctacgcccggcgtgaaatcaagggtgaatgggggagcgcaacctgggaaatgcgctcggtaaaccagcgttatctggaaacttactttcgtctgccggagcagttccgtagccttgaacctgtcgttcgcgagcgtattcgttctcgcctgacgcgcggtaaagtggaatgtaccctgcgctatgagccagatgttagcgcgcaaggtgagctgatcctcaacgaaaaactggctaaacagctggtaactgccgcgaactgggtaaaaatgcagagtgacgaaggggaaatcaacccggttgatattctacgctggccgggcgtgatggcagcccaggagcaggatcttgacgccattgccgctgaaattctcgcggcgctggatggtacgctggacgactttattgtcgcgcgcgaaaccgaaggtcaggcactgaaagcattgatcgagcagcgtctggaaggcgtcaccgccgaagtggtcaaagtccgctcccatatgccggaaatcctgcaatggcagcgtgagcgtctggtcgcgaagctggaagatgctcaggtgcaactggaaaacaaccgtctggagcaggaactggttctgctggcacaacgaattgacgttgccgaagaactggatcgcctcgaagcgcatgtcaaagagacctacaacattctgaagaaaaaagaagcggttggtcgtcgtctggattttatgatgcaggagttcaaccgcgagtcgaacactcttgcgtcgaagtctatcaatgccgaagtgacaaactccgccatcgagctgaaagtgttgattgagcagatgcgcgagcagattcagaacatcgaataa

SEQ ID NO: 43 - DNA, 1293 bp, Escherichia coli
atgcgctataatggtttaaataatatgtttttccctctttgcctgattaacgataaccactctgtcacaagtccatcacatacaaagaaaacaaaatcagataattacagcaaacatcataaaaacacgttaattgacaataaagccctctctcttttcaaaatggatgatcatgaaaaagtgataggcttgattcagaaaatgaaaagaatttatgatagtttaccatcaggaaaaatcacgaaagaaacggacaggaaaatacataaatattttatagatatagcttcacatgcaaataataaatgtgacgatagaattacgagaagagtttaccttaataaagataaggaagtgtcaattaaggtggtatattttataaataatgtcaccgtccataataatactatcgaaatcccacagacagtaaatggtggttacgatttttcacaccttagcctgaaaggtatcgtgattaaagatgaagatttatccaattcgaattttgcaggttgcagactacaaaacgctatttttcaggactgtaatatgtataaaacgaattttaatttcgccataatggaaaaaatactttttgataattgtattctcgatgactcaaatttcgctcagataaaaatgactgacggaactctaaattcatgttccgctatgcatgttcaattctacaatgcaacaatgaatagagccaatattaaaaataccttccttgattattcaaatttttatatggcatacatggctgaggtaaatctttataaagtaatagcgccatatattaatttatttagagccgaccttagcttctctaaacttgatttaattaactttgaacatgctgatctgtctcgtgtcaacctgaataaagcaaccctccagaatataaacttaattgatagcaaactcttttttacgcggttaacaaatacgttcctcgaaatggttatatgtaccgactctaatatggctaatgttaattttaataatgccaatttaagcaattgccatttcaactgttctgttttaacaaaagcctggatgtttaatatccgtctctatcgtgttaatttcgatgaggctagcgtccagggaatgggtattaccattctccgtggtgaggaaaatatctccattaatagtgatatcctggtaacactacagaaattctttgaagaagattgtgccactcatactggcatgtcacaaactgaggataatcttcatgcagtcgctatgaagattactgcagatattatgcaagatgcagattga

SEQ ID NO: 44 - DNA, 489 bp, Escherichia coli
atgagactgacatctaaagggcgctatgccgtgaccgcaatgcttgacgttgcgctcaactctgaagcgggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttgtcagggtaaaggcggctgccagggcggcgataaatgcctgacccacgcgctgtggcgtgatttgagcgaccgtctcaccggttttctcaacaacattactttaggcgaactggttaataaccaggaagtgctggatgtgtctggtcgtcagcatactcacgacgcgccacgcacccgcacacaagacgcgatcgacgttaagttacgcgcttaa

SEQ ID NO: 45 - DNA, 816 bp, Escherichia coli
atgttttcaattcaacaaccactactggtttttagcgatcttgatggcaccctgctggacagtcatagttatgactggcaaccggcagccccctggctcacccgtttacgcgaagcaaatgttcccgtcattctctgtagcagtaaaacatcagcggaaatgctgtacttgcaaaaaacgttggggctacaaggtttaccgctgattgccgaaaatggcgcagtgatccagcttgctgagcaatggcaggagatagacggttttccacgcatcatctcaggtattagccatggcgaaatcagcctggttttaaatacgctacgcgagaaagaacattttaaattcacgacttttgatgatgtcgacgatgcaaccatcgccgaatggacgggattaagccgtagccaggcggcgctgacgcagcttcatgaggcgtcggtaacgctaatctggcgcgacagtgacgagcgtatggcacaatttaccgctcgtctgaacgaactgggcttacagtttatgcagggtgcgcgcttctggcacgtactggatgcttctgccggaaaagatcaggctgccaactggattatcgcgacctatcaacaattgtcaggcaaacgcccaaccacacttggcctgggcgatgggccaaacgatgcgcccttactggaggtaatggattacgcggtgattgtgaaagggctaaatcgtgaaggggtgcatctgcatgatgaggatccggcccgcgtctggcgaacgcagcgtgaaggaccggaaggatggcgtgaagggctggaccattttttctccgcccgttaa

(19) United States
(12) Patent Application Publication
(10) Pub. No.: US 2019/0002935 A1
(43) Pub. Date: Jan. 3, 2019

(54) BACTERIAL CELLS WITH IMPROVED TOLERANCE TO POLYAMINES

(71) Applicant: DANMARKS TEKNISKE UNIVERSITET, Kgs. Lyngby (DK)

(72) Inventors: Rebecca Lennen, Holte (DK); Alex Toftgaard Nielsen, Rungsted Kyst (DK); Markus Herrgård, Virum (DK); Morten Sommer, Virum (DK); Adam Feist, San Diego, CA (US); Elsayed Tharwat Tolba Mohamed, Helsingborg (SE)

(21) Appl. No.: 15/781,999

(22) PCT Filed: Dec. 7, 2016
(86) PCT No.: PCT/EP2016/080059

§ 371 Date: Jun. 6, 2018

(30) Foreign Application Priority Data
Feb. 9, 2016 (EP) 16154829.2

(51) Int. Cl. C12P 13/00; C07K 14/255; C07K 14/245; C12N 9/12; C12N 9/80; C12N 9/16; C12N 9/00

(52) U.S. Cl. CPC C12P 13/001; C07K 14/255; C07K 14/245; C12N 9/1223; C12Y 207/03009; C12Y 603/02; C12Y 305/01014; C12N 9/16; C12Y 301/0307; C12N 9/93; C12N 9/80

(57) ABSTRACT

Provided are bacterial cells genetically modified to improve their tolerance to certain commodity chemicals, such as polyamines, and methods of preparing and using such bacterial cells for production of polyamines and other compounds.

### FIELD OF THE INVENTION

The present invention relates to bacterial cells genetically modified to improve their tolerance to certain commodity chemicals, such as polyamines, and to methods of preparing and using such bacterial cells for production of polyamines and other compounds.

### BACKGROUND OF THE INVENTION

Polyamines (NH2-R—NH2, where R is an alkyl chain) are most commonly used as precursors for nylon polymers (polyamides), which are most typically prepared by condensing polyamines with diacids. Different chain lengths of the constituent polyamines and diacids impart different physical properties to the polymer. These and other bulk chemicals are of special interest to produce from renewable feedstocks via microbial conversion, using either existing or introduced biochemical pathways for producing the chemicals (Chung et al., 2015, Chae et al., 2015; Qian, 2009; Qian 2011).

To develop economically attractive processes for production of bulk chemicals from renewable plant-based carbon feedstocks, three features are essential: high product yields, high productivity, and high product titers. The latter property is particularly important in order to minimize capital equipment and downstream separations costs for product purification. Titers of bulk chemicals in economical fermentation processes often exceed 100 g/L; however, most chemicals at these concentrations (or much lower) exhibit significant toxicity that further reduce yields and productivities by negatively affecting microbial growth.

Escherichia coli being a suitable host for industrial applications, there has been much interest in developing E. coli strains with improved tolerance to chemicals of interest for production, such as, e.g., n-butanol, ethanol and isobutanol, or to stress conditions present during fermentation (see, e.g., Sandberg et al., 2014; Lennen and Herrgård, 2014; Tenaillon et al., 2012; Minty et al., 2011; Dragosits et al., 2013; Winkler et al., 2014; Wu et al., 2014; LaCroix et al., 2015; Jensen et al., 2015; Doukyu et al., 2012; Shenhar et al., 2012; and Rath and Jawali, 2006).

Despite these and other advances in the art, there is still a need for bacterial cells with improved tolerance to chemicals of interest for bio-based production, such as polyamines.

### SUMMARY OF THE INVENTION

It has been found by the present inventors that certain genetic modifications unexpectedly improve the tolerance of bacterial cells, such as those of the Escherichia and Corynebacterium genera, to certain chemical compounds, particularly aliphatic polyamines.

Accordingly, the invention provides bacterial cells with improved tolerance to at least one aliphatic polyamine, as well as bacterial cells which are capable of producing an aliphatic polyamine which has improved tolerance to the aliphatic polyamine. Particularly contemplated are putrescine, hexamethylenediamine (HMDA), cadaverine, spermidine, agmatine, 1,3-diaminopropane, ethylenediamine, citrulline, and ornithine.

Also provided are compositions comprising such bacterial cells and an aliphatic polyamine, methods of preparing or screening for such bacterial cells, and methods of producing aliphatic polyamines using such bacterial cells.

These and other aspects and embodiments are described further below.

### BRIEF DESCRIPTION OF THE DRAWINGS

FIG. 1: Phase contrast microscope images of putrescine and HMDA evolved isolates containing cell wall or cell shape related mutations (A, top), or MAGE-reconstructed mutants (B, bottom). Cultures were grown to exponential phase in M9 medium.

FIG. 2: Normalized tOD1(evolved)/tOD1(wild-type) for putrescine-evolved isolates grown in the presence of inhibitory concentrations of 12 different chemicals.

FIG. 3: Normalized tOD1(evolved)/tOD1(wild-type) for HMDA-evolved isolates grown in the presence of inhibitory concentrations of 12 different chemicals.

### DETAILED DISCLOSURE OF THE INVENTION

Accordingly, various aspects of the invention provide for genetically modified bacterial host cells with a higher tolerance to one or more aliphatic polyamines. When transformed with a recombinant biosynthetic pathway for producing the polyamine from a carbon source, the genetically modified bacterial host cells of the invention result in improved production of the polyamine from carbon feedstock, since they maintain robust metabolic activity in the presence of higher concentrations of the polyamine than the unmodified parent cells.

So, in one aspect the bacterial cell comprises a recombinant biosynthetic pathway for producing an aliphatic polyamine and at least one genetic modification which reduces expression of an endogenous gene selected from the group consisting of proV, proW, proX, cspC, ptsP, wbbK, yobF, nagC, nagA, rph, ybeX and mpl, or a combination of any thereof. The bacterial cell may, for example, comprise a genetic modification which reduces expression of ybeX, proV, cspC, ptsP, wbbK, mpl or rph. Preferably, the genetic modification comprises a knock-down or knock-out of the endogenous gene. In one embodiment, the genetic modification is a knock-out. Optionally, the bacterial cell further comprises a mutation in at least one of YgaC, RpsG, MreB, NusA, SspA, MrdB, RpoD, RpoC, RpoB, MurA, RpsA, SpoT and argG.

In one aspect, the bacterial cell comprises genetic modifications which reduce the expression of at least two endogenous genes selected from the group consisting of proV, proW, proX, cspC, ptsP, wbbK, yobF, nagC, nagA, rph, yicC, yjcF, iscR, yedP, ybeX and mpl. In one embodiment, the bacterial cell comprises genetic modifications which reduce the expression of at least two endogenous genes selected from the group consisting of proV, proW, proX, cspC, ptsP, wbbK, yobF, nagC, nagA, rph, ybeX and mpl. The bacterial cell may, for example, comprise genetic modifications which reduce the expression of proV and at least one of ptsP, wbbK, cspC and yobF. Preferably, the genetic modification comprises a knock-down or knock-out of the endogenous gene. In one embodiment, the genetic modification is a knock-out. In one embodiment, the genetic modification is a knock-out. Optionally, the bacterial cell further comprises a mutation in at least one of YgaC, RpsG, MreB, NusA, SspA, MrdB, RpoD, RpoC, RpoB, MurA, RpsA, SpoT and argG.

In one aspect, the bacterial cell comprises at least one mutated endogenous protein selected from YgaC-R43L, RpsG-L157*, MreB-A298V, MreB-N34K, MreB-E212A, MreB-I24M, MreB-H93N, NusA-L152R, NusA-M204R, SspA-F83C, SspA-V91F, MrdB-E254K, RpoD-E575A, RpoC-V401G, RpoC-V453I, RpoC-R1140C, RpoC-L120P, RpoB-R637L, RpoB-G181V, MurA-G141A, MurA-Y393S, RpsA-D160V, RpsA-D310Y, RpsA-D310G, RpsA-N313K, RpsA-N315K, RpsA-E427R, SpoT-R209H, SpoT-R467H, SpoT-R467L, SpoT-R471H, SpoT-R488C, SpoT-G530C or a C324A mutation in the endogenous gene argG.

In one embodiment of any one of the preceding aspects, the bacterial cell may, for example, comprise a genetic modification which reduces expression of proV or ybeX and at least one mutation or combination of mutations selected from
(i) YgaC-R43L;
(ii) RpsG-L157*;
(iii) RpsG-L157* and MreB-A298V;
(iv) NusA-L152R and SspA-F83C;
(v) MrdB-E254K;
(vi) RpoD-E575A and RpoC-V401G;
(vii) RpoD-E575A, RpoB-R637L, and MurA-Y393S;
(viii) RpsA-D310Y, NusA-M204R, MreB-H93N, and SpoT-R467H; and
(ix) a mutation in rph or the pyrE/rph intergenic region which increases the expression of pyrE.

In a further embodiment of any one of the preceding aspects and embodiments, the genetic modification preferably provides for an increased growth rate, a reduced lag time, or both, of the cell in at least one of putrescine, hexamethylenediamine (HMDA), spermidine, agmatine, 1,3-diaminopropane, cadaverine, ethylenediamine, citrulline, and ornithine.

In a further embodiment of any one of the preceding aspects and embodiments, the bacterial cell comprises a recombinant biosynthetic pathway for producing at least one of putrescine, HMDA, spermidine, agmatine, 1,3-diaminopropane, cadaverine, ethylenediamine, citrulline and ornithine.

In a further embodiment of any one of the preceding aspects and embodiments, the bacterial cell is of the Escherichia or Corynebacterium genus. Preferably, the bacterial cell is of the Escherichia coli species.

In one aspect, there is provided a process for preparing a recombinant bacterial cell, optionally an E. coli cell, for producing a polyamine, comprising genetically modifying the cell to
(i) introduce a recombinant biosynthetic pathway for producing a polyamine;
(ii) knock-down or knock-out at least one endogenous gene selected from the group consisting of proV, proW, proX, cspC, ptsP, wbbK, yobF, nagC, nagA, rph, yicC, yjcF, iscR, yedP, ybeX and mpl, and/or
(iii) introduce at least one mutation selected from YgaC-R43L, RpsG-L157*, MreB-A298V, MreB-N34K, MreB-E212A, MreB-I24M, MreB-H93N, NusA-L152R, NusA-M204R, SspA-F83C, SspA-V91F, MrdB-E254K, RpoD-E575A, RpoC-V401G, RpoC-V453I, RpoC-R1140C, RpoC-L120P, RpoB-R637L, RpoB-G181V, MurA-G141A, MurA-Y393S, RpsA-D160V, RpsA-D310Y, RpsA-D310G, RpsA-N313K, RpsA-N315K, RpsA-E427R, SpoT-R209H, SpoT-R467H, SpoT-R467L, SpoT-R471H, SpoT-R488C, SpoT-G530C and argG-C324A.

In one aspect, there is provided a process for improving the tolerance of an E. coli cell to at least one aliphatic polyamine selected from putrescine, HMDA, spermidine, agmatine, 1,3-diaminopropane, cadaverine, ethylenediamine, citrulline and ornithine, comprising
(i) genetically modifying the cell to knock-down or knock-out at least one endogenous gene selected from the group consisting of proV, proW, proX, cspC, ptsP, wbbK, yobF, nagC, nagA, rph, yicC, yjcF, iscR, yedP, ybeX and mpl;
(ii) preparing a population of E. coli cells comprising one or more mutations in at least one endogenous gene selected from ygaC, rpsG, mreB, nusA, sspA, mrdB, rpoD, rpoC, rpoB, murA, rpsA, spoT and argG; and selecting any host cell which has an improved tolerance to at least one of putrescine, HMDA, 1,3-diaminopropane, cadaverine, spermidine, agmatine, ethylenediamine, citrulline and ornithine; or
(iii) both i) and ii).

In one aspect, there is provided a method for producing an aliphatic polyamine, comprising culturing the bacterial cell of any one of the preceding aspects or embodiments in the presence of a carbon source.

In one aspect, there is provided a composition comprising putrescine, HMDA, spermidine, agmatine, cadaverine, 1,3-diaminopropane, ethylenediamine, citrulline, or ornithine at a concentration of at least 10 g/L, such as at least 25 g/L g/L, and a plurality of bacterial cells of the Escherichia genus which comprise
(i) at least one genetic modification which reduces expression of an endogenous gene selected from the group consisting of proV, proW, proX, cspC, ptsP, wbbK, yobF, nagC, nagA, rph, yicC, yjcF, iscR, yedP, ybeX and mpl, or a combination of any thereof;
(ii) a mutation in at least one of ygaC, rpsG, mreB, nusA, sspA, mrdB, rpoD, rpoC, rpoB, murA, rpsA, spoT and argG which improves the tolerance of the bacterial cell to putrescine, HMDA, spermidine, agmatine, cadaverine, 1,3-diaminopropane, ethylenediamine, citrulline, or ornithine; or
(iii) a combination of a) and b).

### Definitions

An “aliphatic polyamine” as used herein is an organic compound comprising an aliphatic carbon chain to which two or more primary amino (—NH₂) groups are attached, and includes linear aliphatic polyamines and derivatives thereof. Aliphatic polyamines suitable for production in bacteria typically comprise from 2 to 12 carbon atoms, preferably 2 to 10 carbon atoms, more preferably 2 to 8 carbon atoms, and, most preferably, 2 to 6 carbon atoms, and, optionally comprises one or more heteroatoms such as, e.g., 0, N or S. Linear aliphatic polyamines comprising 2, 3 or 4 primary amino groups are preferred and include, but are not limited to, ethylenediamine (1,2-diaminoethane), 1,3-diaminopropane (propane-1,3-diamine), putrescine (butane-1,4-diamine), cadaverine (pentane-1,5-diamine), spermidine (N-(3-aminopropyl)-1,4-diaminobutane, agmatine (1-amino-4-guanidinobutane), spermine (N,N′-bis(3-aminopropyl)-1,4-diaminobutane) and hexamethylenediamine (hexane-1,6-diamine; HMDA), as well as amino acids containing multiple amines, such as, e.g., citrulline, ornithine, carnitine, 2,6-diaminopimelic acid, arginine and lysine. Linear aliphatic diamines having, e.g., 2 to 8 carbon atoms and which do not contain any heteroatoms other than nitrogen (N), such as, e.g., putrescine, HMDA, 1,3-diaminopropane, ethylenediamine, spermidine and cadaverine, and amino acids containing multiple amines, such as, e.g., citrulline and ornithine, are most preferred.

As used herein, a “recombinant biosynthetic pathway” for a compound of interest refers to an enzymatic pathway resulting in the production of a compound of interest in a host cell, wherein at least one of the enzymes is expressed from a transgene, i.e., a gene added to the host cell genome by transformation. In some cases, the recombinant biosynthetic pathway also comprises a deletion of one or more native genes in the host cell. The compound of interest is typically a polyamine, such as an aliphatic polyamine, and may be the actual end product or a precursor or intermediate in the production of another end product.

The terms “tolerant” or “improved tolerance”, when used to describe a genetically modified bacterial cell of the invention or a strain derived therefrom, refers to a genetically modified bacterial cell or strain that shows a reduced lag time, an improved growth rate, or both, in the presence of an aliphatic polyamine than the parent bacterial cell or strain from which it is derived, typically at concentrations of at least 5 g/L, such as at least 10 g/L, such as at least 15 g/L, such as at least 19 g/L, such as at least 20 g/L, such as at least 25 g/L, such as at least 30 g/L, such as at least 35 g/L, such as at least 38 g/L, such as at least 40 g/L. An improved growth rate is at least 5%, such as at least 10%, such as at least 20%, such as at least 50%, such as at least 75% higher than that of a control, typically the parent cell or strain. A reduced lag time is at least 10%, such as at least 20%, such as at least 50%, such as at least 75%, such as at least 90% shorter than that of a control, typically the parent cell or strain.

The term “gene” refers to a nucleic acid sequence that encodes a cellular function, such as a protein, optionally including regulatory sequences preceding (5′ non-coding sequences) and following (3′ non-coding sequences) the coding sequence. An “endogenous gene” refers to a native gene in its natural location in the genome of an organism. A “transgene” is a gene, native or heterologous, that has been introduced into the genome by a transformation procedure.

As used herein the term “coding sequence” refers to a DNA sequence that encodes a specific amino acid sequence.

The term “native”, when used to characterize a gene or a protein herein with respect to a host cell, refers to a gene or protein having the nucleic acid or amino acid sequence as found in the host cell.

The term “heterologous”, when used to characterize a gene or protein with respect to a host cell, refers to a gene or protein which has a nucleic acid or amino acid sequence not normally found in the host cell.

As used herein the term “transformation” refers to the transfer of a nucleic acid fragment, such as a gene, into a host cell. Host cells containing a gene introduced by transformation or a “transgene” are referred to as “transgenic” or “recombinant” or “transformed” cells.

As used herein, a “genetic modification” refers to the introduction a genetically inherited change in the host cell genome. Examples of changes include mutations in genes and regulatory sequences, mutations in coding and non-coding DNA sequences. “Mutations” include deletions, substitutions and insertion of nucleic acids or nucleic acid fragments in the genome.

The term “expression”, as used herein, refers to the process in which a gene is transcribed into mRNA, and may optionally include the subsequent translation of the mRNA into an amino acid sequence, i.e., a protein or polypeptide.

As used herein, “reduced expression” or “downregulation” of an endogenous gene in a host cell means that the levels of the mRNA, protein and/or protein activity encoded by the gene are significantly reduced in the host cell, typically by at least 25%, such as at least 50%, such as at least 75%, such as at least 90%, such as at least 95%, as compared to a control. Typically, when the reduced expression is obtained by a genetic modification in the host cell, the control is the unmodified host cell. Sometimes, e.g., in the case of gene knock-out, the reduction of native mRNA and functional protein encoded by the gene is higher, such as 99% or greater.

“Increased expression”, “upregulation”, “overexpressing” or the like, when used in the context of a protein or activity described herein, means increasing the protein level or activity within a bacterial cell. An up-regulation of an activity can occur through, e.g., increased activity of a protein, increased potency of a protein or increased expression of a protein. The protein with increased activity, potency or expression can be encoded by genes disclosed herein.

Genetic modifications resulting in a reduced expression of a target gene/protein can include, e.g., knock-down of the gene (e.g., a mutation in a promoter that results in decreased gene expression), a knock-out of the gene (e.g., a mutation or deletion of the gene that results in 99 percent or greater decrease in gene expression), a mutation or deletion in the coding sequence which results in the expression of non-functional protein, and/or the introduction of a nucleic acid sequence that reduces the expression of the target gene, e.g. a repressor that inhibits expression of the target or inhibitory nucleic acids (e.g. CRISPR etc.) that reduces the expression of the target gene.

Standard recombinant DNA and molecular cloning techniques used here are well known in the art and are described by Sambrook, J., Fritsch, E. F. and Maniatis, T. Molecular Cloning: A Laboratory Manual, 4^{th} ed.; Cold Spring Harbor Laboratory: Cold Spring Harbor, N.Y., 2012; and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by John Wiley & Sons (1995); and by Datsenko and Wanner, 2000; and by Baba et al., 2006; and by Thomason et al., 2007.

A “conservative” amino acid substitution in a protein is one that does not negatively influence protein activity. Typically, a conservative substitution can be made within groups of amino acids sharing physicochemical properties, such as, e.g., basic amino acids (arginine, lysine and histidine), acidic amino acids (glutamic acid and aspartic acid), polar amino acids (glutamine and asparagines), hydrophobic amino acids (leucine, isoleucine, valine and methionine), aromatic amino acids (phenylalanine, tryptophan and tyrosine), and small amino acids (glycine, alanine, serine, and threonine). Most commonly, substitutions can be made between Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly. Other preferred substitutions are set out in Table 1 below. The following list shows examples of amino acid substitutions:

| Original | | Preferable |
|---|---|---|
| amino acid | Examples of substitutions | substitution |
| Ala (A) | val; leu; ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | Ala | Ala |
| His (H) | asn; gln; lys; arg | Arg |
| Ile (I) | leu; val; met; ala; phe; norleucine | Leu |
| Leu (L) | norleucine; ile ; val; met; ala; phe | Ile |
| Lys (K) | arg; gln; asn | Arg |
| Met (M) | leu; phe; ile | Leu |
| Phe (F) | leu; val; ile; ala; tyr | Tyr |
| Pro (P) | Ala | Ala |
| Ser (S) | thr | Thr |
| Thr (T) | Ser | Ser |
| Trp (W) | tyr; phe | Tyr |
| Tyr (Y) | trp; phe; thr; ser | Phe |
| Val (V) | ile; leu; met; phe; ala; norleucine | Leu |

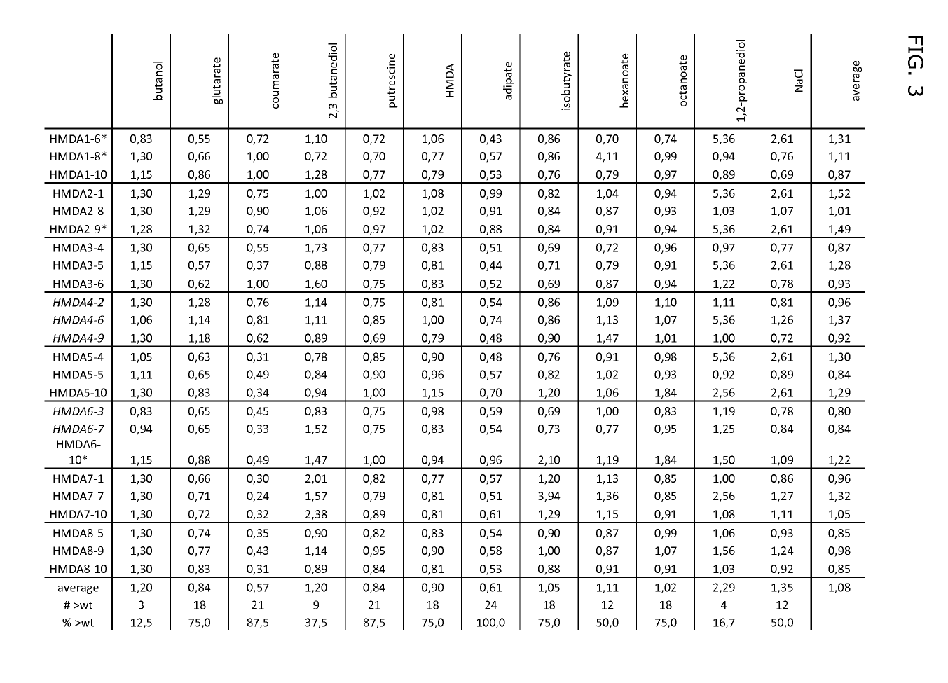

In the numbers in the Tables and FIGS. 3 and 4, a comma represents the decimal mark.

### Specific Embodiments of the Invention

As described in the Examples, the maximum measured concentrations of putrescine and HMDA at which native K-12 MG1655 strain could grow was 40 g/L, respectively, with zero growth detected at 40 g/L and 50 g/L concentrations of putrescine and HMDA, respectively, thus limiting the economic feasibility of production of aliphatic polyamines as platform chemicals. By contrast, bacterial cells comprising one or more mutations according to the invention exhibit a dramatically improved growth at high concentrations of aliphatic polyamines such as putrescine and/or HMDA, e.g., concentrations of 10 g/L or more, such as 25 g/L or more, typically reflected by an increased growth rate, a reduced lag time, or both.

So, provided are bacterial cells with improved tolerance to at least one aliphatic polyamine, such as one or more of putrescine, HMDA, cadaverine, 1,3-diaminopropane, ethylenediamine, spermidine and cadaverine, and amino acids containing multiple amines, such as, e.g., citrulline and ornithine, as well as related processes and materials for producing and using such bacterial cells.

1) Genetic Modifications

The genetic modifications according to the invention include those resulting in reduced expression of genes, e.g., by gene knock-down or knock-out, herein referred to as “Group 1 modifications”; as well as silent mutations in coding or non-coding regions and non-silent (i.e., coding) mutations in coding regions, herein referred to as “Group 2 modifications”; and combinations thereof, as described below.

a) Group 1 Modifications

In one aspect, the bacterial cell has a genetic modification which reduces the expression of one or more endogenous genes selected from the group consisting of proV, proW, proX, cspC, ptsP, wbbK, yobF, nagC, nagA, rph, ybeX and mpl. For example, in one particular embodiment, the one or more endogenous genes are selected from ybeX, proV, cspC, ptsP, wbbK, mpl and rph. In one embodiment, the endogenous gene is selected from ybeX, proV, cspC, ptsP, wbbK, mpl or rph.

In one aspect, there is provided a bacterial cell with improved tolerance to at least one of putrescine, 1,3-diaminopropane, and cadaverine, such as, e.g., to putrescine, comprising a genetic modification which reduces the expression of one or more endogenous genes selected from proV, proW, proX, cspC, ptsP, rph and mpl. In one embodiment, the endogenous gene is selected from one or more of proV, cspC, ptsP, rph and mpl. In one embodiment, the bacterial cell comprises a genetic modification in, e.g., a knock-out or deletion of proV, cspC, or rph. In one embodiment, the bacterial cell comprises a knock-out or deletion of proV; cspC; proV and cspC; proV and ptsP; proV, ptsP and wbbK; proV, ptsP and mpl; or of proV, cspC, and mpl. In another embodiment, the bacterial cell comprises a knock-out, e.g., a deletion, of proV and at least one of ptsP, cspC, and mpl; proV, ptsP, and mpl; and proV, cspC, and mpl.

In one aspect, there is provided a bacterial cell with improved tolerance to at least one of HMDA, spermidine, citrulline, and ornithine, such as, e.g., to HMDA, comprising a genetic modification which reduces the expression of one or more endogenous genes selected from proV, proW, proX, ptsP, wbbK, ybeX, mpl and rph. In one embodiment, the endogenous gene is selected from one or more of proV, ptsP, wbbK, ybeX, mpl and rph. In one embodiment, the bacterial cell comprises a genetic modification in, e.g., a knock-out or deletion of, proV, ptsP, wbbK, ybeX, mpl or rph. In one embodiment, the bacterial cell comprises a knock-out, e.g., a deletion, of proV; ptsP; wbbK; ybeX; mpl; rph; or proV and ptsP, optionally in combination with one or more of wbbK and nagC. In another embodiment, the bacterial cell comprises a knock-out, e.g., a deletion, of ybeX and mpl; proV, ptsP and ybeX; proV, ptsP, ybeX and mpl; proV, cspC and mpl; proV, cspC and ybeX; or of proV, cspC, mpl and ybeX. In another embodiment, the bacterial cell comprises a knock-out or deletion of proV and at least one of ptsP, cspC, mpl, and ybeX; proV, ptsP, and at least one of mpl and ybeX; proV, cspC, and at least one of mpl and ybeX; ybeX and at least one of proV, ptsP, cspC, and mpl; proV, ptsP, ybeX, and mpl; and proV, cspC, ybeX, and mpl.

In one aspect, there is provided a bacterial cell which comprises genetic modifications reducing the expression of at least two endogenous genes selected from the group consisting of proV, proW, proX, cspC, ptsP, wbbK, yobF, nagC, rph, ybeX and mpl. In one embodiment, the bacterial cell comprises a first genetic modification which reduces the expression of a gene selected from ybeX, proV, cspC, ptsP, wbbK, mpl and rph. In one embodiment, the bacterial cell comprises a first genetic modification which reduces the expression of proV and a second genetic modification which reduces the expression of a gene selected from cspC, ptsP, wbbK, yobF, nagC, nagA, rph, ybeX and mpl. In one embodiment, the bacterial cell comprises a first genetic modification which reduces the expression of proW and a second genetic modification which reduces the expression of a gene selected from proV, proX, cspC, ptsP, wbbK, yobF, nagC, nagA, rph, ybeX and mpl. In one embodiment, the bacterial cell comprises a first genetic modification which reduces the expression of proX and a second genetic modification which reduces the expression of a gene selected from proV, proW, cspC, ptsP, wbbK, yobF, nagC, nagA, rph, ybeX and mpl. In one embodiment, the bacterial cell comprises a first genetic modification which reduces the expression of cspC and a second genetic modification which reduces the expression of a gene selected from proV, proW, proX, ptsP, wbbK, yobF, nagC, nagA, rph, ybeX and mpl. In one embodiment, the bacterial cell comprises a first genetic modification which reduces the expression of ptsP and a second genetic modification which reduces the expression of a gene selected from proV, proW, proX, cspC, wbbK, yobF, nagC, nagA, rph, ybeX and mpl. In one embodiment, the bacterial cell comprises a first genetic modification which reduces the expression of wbbK and a second genetic modification which reduces the expression of a gene selected from proV, proW, proX, cspC, ptsP, yobF, nagC, nagA, rph, ybeX and mpl. In one embodiment, the bacterial cell comprises a first genetic modification which reduces the expression of yobF and a second genetic modification which reduces the expression of a gene selected from proV, proW, proX, cspC, ptsP, wbbK, nagC, nagA, rph, ybeX and mpl. In one embodiment, the bacterial cell comprises a first genetic modification which reduces the expression of nagC and a second genetic modification which reduces the expression of a gene selected from cspC, ptsP, wbbK, yobF, rph, ybeX and mpl. In one embodiment, the bacterial cell comprises a first genetic modification which reduces the expression of nagA and a second genetic modification which reduces the expression of a gene selected from cspC, ptsP, wbbK, yobF, nagC, rph, ybeX and mpl. In one embodiment, the bacterial cell comprises a first genetic modification which reduces the expression of rph and a second genetic modification which reduces the expression of a gene selected from cspC, ptsP, wbbK, yobF, nagC, nagA, ybeX and mpl. In one embodiment, the bacterial cell comprises a first genetic modification which reduces the expression of ybeX and a second genetic modification which reduces the expression of a gene selected from cspC, ptsP, wbbK, yobF, nagC, nagA, rph and mpl. In one embodiment, the bacterial cell comprises a first genetic modification which reduces the expression of mpl and a second genetic modification which reduces the expression of a gene selected from cspC, ptsP, wbbK, yobF, nagC, nagA, rph and ybeX. In one specific embodiment, either one or both of the first and second genetic modifications is a knock-out of the gene, optionally a deletion. In an alternative embodiment at least one of the first and second genetic modifications is a knock-down of the gene.

In one aspect, there is provided a bacterial cell according to any one of the preceding aspects and embodiments, wherein the genetic modification is a knock-down of the one or more endogenous genes, resulting in at least 25%, such as at least 50%, such as at least 75%, such as at least 90%, such as at least 95%, reduction in the level of mRNA encoded by the gene.

In one aspect, there is provided a bacterial cell according to any one of the preceding aspects and embodiments, wherein the genetic modification is a knock-down of the one or more endogenous genes, resulting in at least 25%, such as at least 50%, such as at least 75%, such as at least 90%, such as at least 95%, reduction in the level of protein encoded by the gene.

In one aspect, there is provided a bacterial cell according to any one of the preceding aspects and embodiments, wherein the genetic modification is a knock-out of the one or more endogenous genes.

Knock-down or knock-out of a gene can be accomplished by any method known in the art for bacterial cells, and include, e.g., lambda Red mediated recombination, P1 phage transduction, and single-stranded oligonucleotide recombineering/MAGE technologies (see, e.g., Datsenko and Wanner, 2000; Thomason et al., 2007; Wang et al., 2009). Typically, a knock-down of a gene can be accomplished by, for example, a mutation in the promoter region resulting in decreased transcription, a deletion or mutation in the coding region of the gene resulting in a reduced activity of the protein, or by the presence of antisense sequences that interfere with transcription or translation of the gene, resulting in reduced expression of the protein. Preferably, the knocking-down of a gene results in at least 20% reduction in the expression level of the gene product in the bacterial cell, such as at least 30%, such as at least 40%, such as at least 50%, such as at least 60%, such as at least 70%, such as at least 80%, such as at least 90%, such as at least 95% or higher.

A knock-out of a gene includes elimination of a gene's expression, such as by introducing a mutation in the coding sequence and/or promoter so that at least a portion (up to and including all) of the coding sequence and/or promoter is disrupted or deleted deletion, mutation, or insertion, resulting in loss of expression of the protein, or expression only of a non-functional mutant or non-functional fragment of the endogenous protein. As used herein, the symbol “DELTA” denotes a deletion of an endogenous gene. Preferably, a knock-out of a gene results in 1% or less of the gene product being detectable, such as no detectable gene product.

In one aspect, the bacterial cell of any aspect or embodiment described herein comprises a mutation in at least one of YgaC, RpsG, MreB, NusA, SspA, MrdB, RpoD, RpoC, RpoB, MurA, RpsA, SpoT and argG which provides for improved tolerance to at least one aliphatic polyamine, such as one or more of putrescine, HMDA, cadaverine, 1,3-diaminopropane, spermidine, agmatine, ethylenediamine, citrulline, and ornithine. The mutated protein can be expressed from a mutated version of the endogenous gene, or from a transgene. Advantageously, these mutations can be combined with each other and/or with one or more modifications described in the preceding sections.

b) Group 2 Modifications

In one embodiment, the bacterial cell comprises a mutation in YgaC which increases tolerance to at least one of putrescine, HMDA, 1,3-diaminopropane, cadaverine, spermidine, ethylenediamine, citrulline and ornithine. In one particular embodiment, the YgaC comprises a mutation, such as a deletion or amino acid substitution, in residue R43. Preferably, the mutation is R43L or a conservative amino acid substitution thereof. In one particular embodiment, the bacterial cell further comprises at least one Group 1 modification, an additional Group 2 modification, or both, according to any aspects or embodiments herein. For example, the Group 1 modification can be a genetic modification which reduces the expression of proV, proW, proX, ybeX, or mpl, such as proV, and the Group 2 modification a mutation in one or more of RpsG, MreB, NusA, SspA, MrdB, RpoD, RpoC, RpoB, MurA, RpsA, SpoT and argG. In one embodiment, where the bacterial cell comprises a Group 1 modification which reduces the expression of ybeX, the aliphatic diamine is not putrescine, 1,3-diaminopropane, or ethylenediamine.

In one embodiment, the bacterial cell comprises a mutation in RpsG which increases tolerance to putrescine, HMDA, 1,3-diaminopropane, cadaverine, spermidine, ethylenediamine, citrulline, ornithine, or any combination thereof. In one particular embodiment, the RpsG comprises a mutation, such as a coding mutation or amino acid substitution, in residue L157 or W156. Preferably, the mutation is a coding mutation that introduces a translation stop codon. In one particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW proX, ybeX, or mpl, such as proV, and the Group 2 modification a mutation in one or more of YgaC, MreB, NusA, SspA, MrdB, RpoD, RpoC, RpoB, MurA, RpsA, SpoT and argG, such as argG or MreB. In one particular embodiment, the additional Group 2 modifications do not consist of only an R471H mutation in SpoT.

In one embodiment, the bacterial cell comprises a mutation in MreB which increases tolerance to putrescine, HMDA, 1,3-diaminopropane, cadaverine, ethylenediamine, ornithine or combinations thereof. In one particular embodiment, the MreB comprises a mutation, such as a deletion or amino acid substitution, in residue A298. Preferably, the mutation is an A298V, N34K, E212A, I24M, or H93N substitution or a conservative substitution thereof. In one particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, RpoB, MurA, RpsA, SpoT and argG, such as RpsG.

In one embodiment, the bacterial cell comprises a mutation in NusA or NusG which increases tolerance to at least one of putrescine, HMDA, 1,3-diaminopropane, cadaverine, spermidine, citrulline and ornithine. In one particular embodiment, the NusA comprises a mutation, such as a deletion or amino acid substitution, in residue L152. Preferably, the mutation is an L152R or M204R substitution or a conservative substitution thereof. In one particular embodiment, the NusG comprises a mutation, such as a deletion or amino acid substitution, in residue G166, such as G166V. In one embodiment, the bacterial cell further comprises an additional Group 2 modification, such as a mutation SspA, such as SspA-F83C or a conservative substitution thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, RpoB, MurA, RpsA, SpoT and argG, such as SspA.

In one embodiment, the bacterial cell comprises a mutation in SspA which increases tolerance to at least one of putrescine, HMDA, 1,3-diaminopropane, cadaverine, spermidine, ethylenediamine, citrulline and ornithine. In one particular embodiment, the SspA comprises a mutation, such as a deletion or amino acid substitution, in residue F83 or V91. Preferably, the mutation is an F83C or V91F substitution or a conservative substitution thereof. In one embodiment, the bacterial cell further comprises an additional Group 2 modification, such as a mutation NusA, such as NusA-L152R or NusA-M204R, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, RpoB, MurA, RpsA, SpoT and argG, such as NusA.

In one embodiment, the bacterial cell comprises a non-coding mutation in argG which increases tolerance to at least one of putrescine, HMDA, 1,3-diaminopropane, cadaverine, spermidine, ethylenediamine, citrulline and ornithine, for example a cytidine (C) to adenosine (A) substitution in position 324 of the nucleic acid sequence, i.e., in the codon corresponding to amino acid residue A108. In one particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, RpoB, MurA, RpsA, SpoT and argG, such as RpsG.

In one embodiment, the bacterial cell comprises a mutation in MrdB which increases tolerance to at least one of putrescine, HMDA, 1,3-diaminopropane, cadaverine, spermidine, ethylenediamine, citrulline, ornithine. In one particular embodiment, the MrdB comprises a mutation, such as a deletion or amino acid substitution, in residue E254. Preferably, the mutation is an E254K substitution or a conservative substitution thereof. In one embodiment, the bacterial cell further comprises an additional Group 2 modification, such as a mutation in RpoB or RpsA, such as RpoB-R637L or RpsA-D160V, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, RpoD, RpoC, RpoB, MurA, RpsA, SpoT and argG, such as RpoB.

In one embodiment, the bacterial cell comprises a mutation in RpoD which increases tolerance to at least one of putrescine, HMDA, 1,3-diaminopropane, cadaverine, spermidine, ethylenediamine, citrulline and ornithine. In one particular embodiment, the RpoD comprises a mutation, such as a deletion or amino acid substitution, in residue E575. Preferably, the mutation is an E575A substitution or a conservative substitution thereof. In one embodiment, the bacterial cell further comprises an additional Group 2 modification, such as a mutation in RpoC, such as RpoC-V401G, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoC, RpoB, MurA, RpsA, SpoT and argG, such as RpoC.

In one embodiment, the bacterial cell comprises a mutation in RpoC which increases tolerance to at least one of putrescine, HMDA, 1,3-diaminopropane, cadaverine, spermidine, ethylenediamine, citrulline and ornithine. In one particular embodiment, the RpoC comprises a mutation, such as a deletion or amino acid substitution, in residue V401, V453, R1140, or L120. Preferably, the mutation is a V401G, V453I, R1140C, or L120P substitution or a conservative substitution thereof. In one embodiment, the bacterial cell further comprises an additional Group 2 modification, such as a mutation in RpoC, such as RpoD-E575A, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoB, MurA, RpsA, SpoT and argG, such as RpoD.

In one embodiment, the bacterial cell comprises a mutation in RpoB which increases tolerance to at least one of putrescine, HMDA, 1,3-diaminopropane, cadaverine, spermidine, ethylenediamine, citrulline and ornithine. In one particular embodiment, the RpoB comprises a mutation, such as a deletion or amino acid substitution, in residue R637 or G181. Preferably, the mutation is an R637L or G181V substitution or a conservative substitution thereof. In one embodiment, the bacterial cell further comprises an additional Group 2 modification, such as a mutation in RpoC, such as MurA-Y393S, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, MurA, RpsA, SpoT and argG, such as MurA.

In one embodiment, the bacterial cell comprises a mutation in MurA which increases tolerance to at least one of putrescine, HMDA, 1,3-diaminopropane, cadaverine, spermidine, ethylenediamine, citrulline and ornithine. In one particular embodiment, the MurA comprises a mutation, such as a deletion or amino acid substitution, in residue G141 or Y393. Preferably, the mutation is an G141A or Y393S substitution or a conservative substitution thereof. In one embodiment, the bacterial cell further comprises an additional Group 2 modification, such as a mutation in RpoD, such as RpoD-E575A, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, RpoB, RpsA, SpoT and argG, such as RpoD.

In one embodiment, the bacterial cell comprises a mutation in RpsA which increases tolerance to at least one of putrescine, HMDA, 1,3-diaminopropane, cadaverine, spermidine, ethylenediamine, citrulline and ornithine. In one particular embodiment, the RpsA comprises a mutation, such as a deletion or amino acid substitution, in residue D160, D310, N313, N315, or E427. Preferably, the mutation is a D160V, D310Y, D310G, N313K, N315K, or E427R substitution or a conservative substitution thereof. In one embodiment, the bacterial cell further comprises an additional Group 2 modification, such as a mutation in NusA, such as NusA-M204R, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, RpoB, MurA, SpoT and argG, such as NusA.

In one embodiment, the bacterial cell comprises a mutation in SpoT which increases tolerance to at least one of putrescine, HMDA, 1,3-diaminopropane, cadaverine, spermidine, ethylenediamine, citrulline and ornithine. In one particular embodiment, the SpoT comprises a mutation, such as a deletion or amino acid substitution, in residue R209, R467, R471, R488 or G530. Preferably, the mutation is a R209H, R467H, R467L, R471H, R488C, or G530C substitution or a conservative substitution thereof. In one embodiment, the bacterial cell further comprises an additional Group 2 modification, such as a mutation in MreB, such as MreB-E212A, MreB-I24M, MreB-H93N, MreB-A298V, or conservative substitutions thereof. In another particular embodiment, the bacterial cell further comprises at least one Group 1 modification, at least one additional Group 2 modification, or both, according to any aspects or embodiments herein. For example, the Group 1 modification can be a genetic modification which reduces the expression of proV, proW, proX, ybeX, or mpl, such as proV, and the Group 2 modification can be a mutation in one or more of YgaC, MreB, NusA, SspA, MrdB, RpoD, RpoC, RpoB, MurA, RpsA and argG, such as MreB. In one particular embodiment, the additional Group 2 modifications do not consist of only an L157* or W156* mutation in RpsG. In another particular embodiment, the bacterial cell comprises a mutation in SpoT and one or more further genetic modifications.

In separate and specific embodiments, the bacterial cell comprises
a) a genetic modification which reduces expression of proV, and at least one mutation selected from RpsG-L157* and MreB-A298V, optionally wherein the aliphatic polyamine is selected from putrescine, 1,3-diaminopropane, cadaverine, spermidine, citrulline and ornithine;
b) a genetic modification which reduces expression of proV, and mutations RpsG-L157* and MreB-A298V, optionally wherein the aliphatic polyamine is selected from putrescine, 1,3-diaminopropane, cadaverine, spermidine, citrulline and ornithine;
c) a genetic modification which reduces expression of proV, and a mutation YgaC-R43L, optionally wherein the aliphatic polyamine is selected from putrescine, 1,3-diaminopropane, cadaverine, spermidine, citrulline and ornithine;
d) a genetic modification which reduces expression of ybeX, and at least one of proV, ptsP, cspC, and mpl, and at least one mutation selected from SspA-F83C, NusA-L152R, RpsG-L157*, YgaC-R43L, and MreB-A298V, optionally wherein the aliphatic polyamine is selected from HMDA, 1,3-diaminopropane, cadaverine, spermidine, ethylenediamine, citrulline, and ornithine;
e) a genetic modification which reduces expression of ybeX and mpl, and mutations NusA-L152R and SspA-F83C, optionally wherein the aliphatic polyamine is selected from HMDA, 1,3-diaminopropane, cadaverine, spermidine, ethylenediamine, citrulline, and ornithine;
f) a genetic mutation which reduces expression of ybeX and mpl, and mutations RpsG-L157* and MreB-A298V, optionally wherein the aliphatic polyamine is selected from HMDA, 1,3-diaminopropane, cadaverine, spermidine, ethylenediamine, citrulline, and ornithine;
g) a genetic mutation which reduces expression of ybeX and mpl, and mutation YgaC-R43L, optionally wherein the aliphatic polyamine is selected from HMDA, 1,3-diaminopropane, cadaverine, spermidine, ethylenediamine, citrulline, and ornithine.

In an alternative embodiment, the bacterial cell comprises an upregulation of at least one of YgaC, RpsG, MreB, NusA, SspA, MrdB, RpoD, RpoC, MurA, RpsA, SpoT and argG, e.g., by transforming the bacterial cell with a transgene expressing the endogenous protein or a functionally active variant thereof, e.g., RpsG-L157*, MreB-A298V, MreB-N34K, MreB-E212A, MreB-I24M, MreB-H93N, NusA-L152R, NusA-M204R, SspA-F83C, SspA-V91F, MrdB-E254K, RpoD-E575A, RpoC-V401G, RpoC-V453I, RpoC-R1140C, RpoC-L120P, RpoB-R637L, RpoB-G181V, MurA-G141A, MurA-Y393S, RpsA-D160V, RpsA-D310Y, RpsA-D310G, RpsA-N313K, RpsA-N315K, RpsA-E427R, SpoT-R209H, SpoT-R467H, SpoT-R467L, SpoT-R471H, SpoT-R488C, SpoT-G530C and argG-C324A. To cause an up-regulation through increased expression of a protein, the copy number of a gene or genes encoding the protein may be increased. Alternatively, a strong and/or inducible promoter can be used to direct the expression of the gene, the gene being expressed either as a transient expression vehicle or homologously or heterologously incorporated into the bacterial genome. In another embodiment, the promoter, regulatory region and/or the ribosome binding site upstream of the gene can be altered to achieve the over-expression. The expression can also be enhanced by increasing the relative half-life of the messenger or other forms of RNA. Any one or a combination of these approaches can be used to effect upregulation of a desired target protein as needed.

In one embodiment, the bacterial cell comprises one or more mutations which increase(s) the expression level or activity of PyrE. E. coli K-12 MG1655 and W3110, plus their common ancestor strain W1485, are known to exhibit pyrimidine starvation in minimal media due to the presence a frameshift mutation occurring in rph relative to other E. coli strains (Jensen et al., 1993). This mutation disrupts the transcriptional/translational coupling required for efficient translation of pyrE, encoding orotate phosphoribosyltransferase in the pyrimidine biosynthesis pathway. Compensatory mutations that correct this deficiency are well-known in the art. One of these mutations is an 82 bp deletion near the 3′ terminus of rph, due to presence of two homologous GCAGAAGGC sequences flanking this 82 bp region (Conrad et al., 2009). This mutation precisely corresponds to the 82 bp deletion found in resequenced isolates from populations HMDA4 and HMDA6, and isolates PUTR6-7 and PUTR6-10 (from NC_000913.3 coordinates 3815859 to 3815931; Table 4). In addition to the 82 bp deletion, a 1 bp deletion at coordinate 3815809 in the pyrE/rph intrgenic region has previously been encountered in strains evolved for growth on a minimal glucose medium (LaCroix et al., 2015), and a wide array of other frameshift mutations, substitutions, and coding mutations near the 3′ terminus of rph were encountered in a short-term selection/evolution of combinatorial mutant libraries in minimal medium at an elevated temperature of 42° C. (Sandberg et al., 2014). The same 1 bp deletion in the pyrE/rph intergenic region was also found to be present in evolved isolates HMDA3-4, HMDA3-5, HMDA3-6, HMDA5-4, HMDA5-5, and HMDA5-10. Another 1 bp deletion in the pyrE/rph intergenic region was found at coordinate 3815801 in evolved isolates PUTR8-3, PUTR8-6, PUTR8-10, HMDA2-1, HMDA2-8, HMDA8-5, HMDA8-9, and HMDA8-10. Furthermore, intergenic mutations between rph and yicC at coordinate 3816611 (C to A mutation) were found in resequenced isolates from population PUTR3 and HMDA1. Without being limited to theory, all of these mutations can serve the same function of increasing expression of PyrE, with the selective pressure for these mutations being even stronger in minimal media with particular imposed stresses (certain chemicals or heat) than in minimal media alone. In one embodiment, the bacterial cell comprises mutations in rph or the pyrE/rph intergenic region, such as, e.g., an 82 bp deletion near the 3′ terminus of rph, an intergenic C to A mutation at coordinate 3816611 in the intergenic region between rph and yicC, or 1 or 82 bp deletions in the intergenic region between pyrE and rph.

2) Production Pathways

In one aspect, there is provided a bacterial cell with improved tolerance to at least one aliphatic polyamine according to any aspect or embodiment described herein, wherein the bacterial cell further comprises a recombinant biosynthetic pathway for producing an aliphatic polyamine of interest, such as, e.g., putrescine, HMDA, spermidine, agmatine, cadaverine, 1,3-diaminopropane, citrulline or ornithine. In principle, any such recombinant biosynthetic pathway which is known in the art can be introduced into the cell by standard recombinant technologies. Some specific, preferred pathways are, however, exemplified below and in Example 1, section I). Preferably, the bacterial cell comprising the recombinant biosynthetic pathway produces at least 2 times, at least three times, at least 5 times or at least 10 times or more of the aliphatic polyamine than the wild-type bacterial cell during a predetermined time period, e.g., 24 h or more, under the same conditions, i.e., conditions suitable for producing the aliphatic polyamine. It is to be understood that, when a specific enzyme of these biosynthetic pathways is mentioned by name such as, e.g., “acetylglutamate kinase”, the enzyme may be any characterized and sequenced enzyme, from any species, that have been reported in the literature so long as it provides the desired activity. In some embodiments, the enzyme is an overexpressed gene which is native to the host cell used. In some embodiments, the enzyme is a functionally active fragment or variant of an enzyme which is heterologous or native to the host cell. Also, in some embodiments, the recombinant biosynthetic pathway comprises a knock-down or a knock-out of one or more genes, typically for the purpose of avoiding competing reactions reducing the yield of the desired aliphatic polyamine.

So, in one embodiment, the biosynthetic pathway is for producing putrescine and comprises overexpressed or de-regulated N-acetylglutamate kinase (ArgB; EC 2.7.2.8), N-acetylglutamylphosphate reductase (ArgC; EC 1.2.1.38), N-acetylornithine aminotransferase/N-succinyldiaminopimelate aminotransferase (ArgD; EC 2.6.1.11), acetylornithine deacetylase (ArgE; EC 3.5.1.16), putrescine:H⁺ symporter/putrescine:ornithine antiporter (PotE), and ornithine decarboxylate (SpeC and/or SpeF; EC 4.1.1.17), and a knock-down or knock-out of any native ornithine carbamoyltransferase (ArgI and/or ArgF; EC 2.1.3.3), spermidine synthase (SpeE; EC 2.5.1.16), spermidine acetyltransferase (SpeG; EC 2.3.1.57), glutamate-putrescine ligase (PuuA; EC 6.3.1.11), putrescine:H⁺ symporter (PuuP), and RNA polymerase sigma S (sigma 38) factor (RpoS). In a preferred embodiment, the pathway additionally comprises an overexpressed or de-regulated N-acetylglutamate synthase (ArgA; EC 2.3.1.1). In a preferred embodiment, the bacterial cell is an E. coli cell and comprises overexpressed N-acetylglutamate kinase (ArgB; EC 2.7.2.8), N-acetylglutamylphosphate reductase (ArgC; EC 1.2.1.38), N-acetylornithine aminotransferase/N-succinyldiaminopimelate aminotransferase (ArgD; EC 2.6.1.11), acetylornithine deacetylase (ArgE; EC 3.5.1.16), putrescine:H⁺ symporter/putrescine:ornithine antiporter (PotE), and ornithine decarboxylate (SpeC and/or SpeF; EC 4.1.1.17), and a knock-down or knock-out of any native ornithine carbamoyltransferase (ArgI and/or ArgF; EC 2.1.3.3), spermidine synthase (SpeE; EC 2.5.1.16), spermidine acetyltransferase (SpeG; EC 2.3.1.57), glutamate-putrescine ligase (PuuA; EC 6.3.1.11), putrescine:H⁺ symporter (PuuP), and RNA polymerase sigma S (sigma 38) factor (RpoS) (Qian et al., 2009).

In one embodiment, the biosynthetic pathway is for producing putrescine and comprises overexpressed or de-regulated N-acetylglutamate synthase (ArgA; EC 2.3.1.1), N-acetylglutamate kinase (ArgB; EC 2.7.2.8), N-acetylglutamylphosphate reductase (ArgC; EC 1.2.1.38), N-acetylornithine aminotransferase/N-succinyldiaminopimelate aminotransferase (ArgD or GabT; EC 2.6.1.11), acetylornithine deacetylase (ArgE; EC 3.5.1.16), ornithine carbamoyltransferase (ArgF or ArgI; EC 2.1.3.3), arginosuccinate synthase (ArgG; EC 6.3.4.5), arginosuccinate lyase (ArgH; EC 4.3.2.1), arginine decarboxylase (SpeA; EC 4.1.1.19), ornithine decarboxylase (SpeC; EC 4.1.1.17), agmatinase (SpeB; EC 3.5.3.11), putrescine:H⁺ symporter/putrescine:ornithine antiporter (PotE), and knock-down or knock-out of spermidine synthase (SpeE; EC 2.5.1.16), putrescine:H⁺ symporter (PuuP), and glutamate-putrescine ligase (PuuA; EC 6.3.1.11).

In one embodiment, the biosynthetic pathway is for producing cadaverine and comprises an overexpressed lysine decarboxylase (EC 4.1.1.18) and a knock-down or knockout of any native spermidine synthase (SpeE; EC 2.5.1.16), spermidine acetyltransferase (SpeG; EC 2.3.1.57), glutamate-putrescine ligase (PuuA; EC 6.3.1.11), putrescine:H⁺ symporter (PuuP), and putrescine/cadaverine aminotransferase (YgjG). In a preferred embodiment, the bacterial cell is an E. coli cell and comprises overexpressed lysine decarboxylase (CadA; EC 4.1.1.18) and a knock-down or knock-out of spermidine synthase (SpeE; EC 2.5.1.16), spermidine acetyltransferase (SpeG; EC 2.3.1.57), glutamate-putrescine ligase (PuuA; EC 6.3.1.11), putrescine:H⁺ symporter (PuuP), and putrescine/cadaverine aminotransferase (YgjG) (Qian et al., 2011).

In one embodiment, the biosynthetic pathway is for producing HMDA and comprises expression of 3-oxoadipyl-CoA thiolase (PaaJ; EC 2.3.1.174), 3-oxoadipyl-CoA reductase (PaaH), 3-hydroxyadipyl-CoA dehydratase (MaoC), 5-carboxy-2-pentenoyl-CoA reductase (Bcd and EtfAB), adipyl-CoA reductase (aldehyde forming) (Acr1), 6-aminocaproyl-CoA synthase (GabT), 6-aminocaproic acid transaminase (BioW), and hexamethylenediamine transaminase (YgjG) (US 2012/0282661 A1; e.g., Example XVII).

In one embodiment, the biosynthetic pathway is for producing 1,3-diaminopropane and comprises overexpressed aspartate aminotransferase (AspC; EC 2.6.1.1) and phosphoenolpyruvate carboxylase (Ppc; EC 4.1.1.31), a knock-down or knockout of any native 6-phosphofructokinase I (PfkA; EC 2.7.1.-), and expressing mutated versions of aspartate kinase (ThrA; EC 2.7.2.4) and asparate kinase III (LysC; EC 2.7.2.4) that exhibit removal of feedback inhibition.

In one embodiment, the biosynthetic pathway is for producing spermidine and comprises overexpressed or de-regulated N-acetylglutamate synthase (ArgA; EC 2.3.1.1), N-acetylglutamate kinase (ArgB; EC 2.7.2.8), N-acetylglutamylphosphate reductase (ArgC; EC 1.2.1.38), N-acetylornithine aminotransferase/N-succinyldiaminopimelate aminotransferase (ArgD or GabT; EC 2.6.1.11), acetylornithine deacetylase (ArgE; EC 3.5.1.16), ornithine carbamoyltransferase (ArgF or ArgI; EC 2.1.3.3), arginosuccinate synthase (ArgG; EC 6.3.4.5), arginosuccinate lyase (ArgH; EC 4.3.2.1), arginine decarboxylase (SpeA; EC 4.1.1.19), ornithine decarboxylase (SpeC; EC 4.1.1.17), adenosylmethionine decarboxylase (SpeD; EC 4.1.1.50) and spermidine synthase (SpeE; EC 2.5.1.18), and knock-down or knockout of putrescine:H⁺ symporter (PuuP), glutamate-putrescine ligase (PuuA; EC 6.3.1.11), and spermidine acetyltransferase (SpeG; EC 2.3.1.57).

In one embodiment, the biosynthetic pathway is for producing ornithine and comprises overexpressed or de-regulated (e.g. via knock-down or knockout of ArgR transcriptional dual regulator) N-acetylglutamate synthase (ArgA; EC 2.3.1.1), N-acetylglutamate kinase (ArgB; EC 2.7.2.8), N-acetylglutamylphosphate reductase (ArgC; EC 1.2.1.38), N-acetylornithine aminotransferase/N-succinyldiaminopimelate aminotransferase (ArgD or GabT; EC 2.6.1.11), and acetylornithine deacetylase (ArgE; EC 3.5.1.16), and knock-down or knockout of ornithine carbamoyltransferase (ArgF or ArgI; EC 2.1.3.3) and glutamate 5-kinase (ProB; EC 2.7.2.11) (Hwang et al., 2008)

In one embodiment, the biosynthetic pathway is for producing citrulline and comprises overexpressed or de-regulated (e.g. via knock-down or knockout of ArgR transcriptional dual regulator) N-acetylglutamate synthase (ArgA; EC 2.3.1.1), N-acetylglutamate kinase (ArgB; EC 2.7.2.8) or a feedback-resistant mutant thereof, N-acetylglutamylphosphate reductase (ArgC; EC 1.2.1.38), N-acetylornithine aminotransferase/N-succinyldiaminopimelate aminotransferase (ArgD or GabT; EC 2.6.1.11), and acetylornithine deacetylase (ArgE; EC 3.5.1.16), and ornithine carbamoyltransferase (ArgF or ArgI; EC 2.1.3.3), and knock-down or knockout of arginosuccinate synthase (ArgG; EC 6.3.4.5) (Eberhardt et al., 2014).

Additional production pathways that have been employed in microorganisms for the overproduction of putrescine, cadaverine, ornithine, and citrulline are reviewed in Wendisch et al. (2016), hereby incorporated by reference in its entirety.

3) Processes

In one aspect, there is provided a process for preparing a recombinant bacterial cell, e.g., an E. coli cell. Also provided is a process for improving the tolerance of a bacterial cell, e.g., an E. coli cell, to at least one aliphatic polyamine, such as, e.g., putrescine, HMDA, 1,3-diaminopropane, cadaverine, ethylenediamine, spermidine, citrulline, and ornithine. Also provided is a method of identifying a bacterial cell which is tolerant to at least one such aliphatic polyamine. Also provided is a process for preparing a recombinant bacterial cell, e.g., an E. coli cell, for producing such an aliphatic polyamine.

These processes may comprise one or more steps of genetically modifying a bacterial cell to knock-down or knock-out one or more endogenous genes of any aspect or embodiment of the Group 1 modifications and/or introducing one or more mutations in the endogenous protein(s) or gene(s) of any Group 2 aspect or embodiment. This can be achieved by, e.g., transforming the bacterial cell with genetic constructs, e.g., vectors, antisense nucleic acids or siRNA, which effect the knock-out or knock-down or which introduce the mutation into the endogenous gene or encode the mutated protein from a transgene.

The genetic constructs, particularly vectors, can also comprise suitable regulatory sequences, typically nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters (e.g., constitutive promoters or inducible promoters), translation leader sequences, introns, polyadenylation recognition sequences, RNA processing sites, effector binding sites and stem-loop structures.

Alternatively, bacterial cells can be exposed to selection pressure (as described in the Examples) or to conditions which introduce random mutations in endogenous genes, and bacterial cells which comprise one or more Group 1 and/or Group 2 modifications according to any preceding aspects and embodiments can be identified.

In one specific embodiment, the Group 1 modification is a knock-down or knock-out of one or more endogenous genes selected from proV, proW, proX, cspC, ptsP, wbbK, yobF, nagC, nagA, rph, ybeX and mpl. In one specific embodiment, the Group 2 modification is a mutation in at least one endogenous protein or gene selected from YgaC, RpsG, MreB, NusA, SspA, and argG, such as YgaC-R43L, RpsG-L157*, MreB-A298V, NusA-L152R, SspA-F83C, MrdB-E254K, RpoD-E575A, RpoC-V401G, RpoB-R637L, MurA-Y393S, RpsA-D310Y, NusA-M204R, MreB-H93N, SpoT-R467H and argG-C324A.

The processes may further comprise
a step of selecting any bacterial cell which has an improved tolerance to at least one aliphatic polyamine, e.g., putrescine, HMDA, 1,3-diaminopropane, cadaverine, ethylenediamine, spermidine, agmatine, citrulline or ornithine at a predetermined concentration, such as at least 25 g/L or higher;
a step of introducing a recombinant biosynthetic pathway for producing a polyamine, such as, e.g., putrescine, HMDA, 1,3-diaminopropane, cadaverine, ethylenediamine, spermidine, agmatine, citrulline and/or ornithine; or
both of the above steps, in any order.

Also provided is a method of producing an aliphatic polyamine, comprising culturing the bacterial cell obtained by any one of these methods, or the bacterial cell of any preceding aspect or embodiment, under conditions where the aliphatic polyamine is produced. Typically, these conditions include the presence of a suitable carbon source or mixes of different suitable carbon sources. Non-limiting examples of suitable carbon sources include, e.g., sucrose, D-glucose, D-xylose, L-arabinose, glycerol, as well as hydrolysates produced from cellulosic or lignocellulosic materials. For further details see, e.g., Qian et al., 2009 or 2011.

4) Compositions

A bacterial cell which have an increased tolerance to aliphatic polyamines such as, e.g., putrescine, HMDA, spermidine, agmatine, cadaverine, 1,3-diaminopropane, ethylenediamine, citrulline or ornithine can be useful for the production of such aliphatic polyamines.

In one aspect, there is provided a composition comprising
an aliphatic polyamine at a concentration of at least 5 g/L, such as at least 15 g/L, such as at least 19 g/L, such as at least 20 g/L, such as at least 25 g/L, such as at least 30 g/L, such as at least 35 g/L, such as at least 38 g/L, such as at least 40 g/L; and
a plurality of bacterial cells according to any preceding aspect or embodiment.

Preferably, the bacterial cells are of the Escherichia, Bacillus, Ralstonia, Pseudomonas or Corynebacterium family, such as, e.g., E. coli cells, and comprise

a) at least one genetic modification which reduces expression of an endogenous gene selected from the group consisting of proV, proW, proX, cspC, ptsP, wbbK, yobF, nagC, nagA, rph, ybeX and mpl, or a combination of any thereof;

b) a mutation in at least one of ygaC, rpsG, mreB, nusA, sspA, mrdB, rpoD, rpoC, rpoB, murA, rpsA, spoT and argG which improves the tolerance of the bacterial cell to putrescine, HMDA, cadaverine, 1,3-diaminopropane, spermidine, agmatine, ethylenediamine, citrulline or ornithine; or

c) a combination of a) and b).

5) Bacterial Cells

Also provided are strains, clones and other progeny of the bacterial cells of these and other aspects and embodiments. Typically, as used herein, a “strain” typically refers to a group of cells which are descendants of a initial single colony of parent cells whereas a “clone” is a group of cells which are the descendants of an initial genetically modified single parent cell.

Non-limiting examples of bacterial cells suitable for modification according to any one of the aspects and embodiments described herein include bacteria of the Enterobacteriaceae or Corynebacteriaceae families, particularly the Escherichia, Bacillus, Ralstonia, Pseudomonas and Corynebacterium genera. In one embodiment, the bacterial cell is an E. coli cell, such as a cell of the commercially available and/or fully characterized strains K-12 MG1655, B, BLR, BW25113, BL21, BL21(DE3), K-12 W3110, W, JM109, JM110, REL606, DH1, DH5α, DH10B, C600, S17-1, HB101 or Crooks (ATCC 8739). In another embodiment, the bacterial cell is a Bacillus cell, such as a cell of the commercially available and/or fully characterized strains Bacillus subtilis 168 and Bacillus subtilis PY79. In one embodiment, the bacterial cell is a Pseudomonas cell, such as a cell of the commercially available and/or fully characterized strain Pseudomonas putida KT2440. In another embodiment, the bacterial cell is a Ralstonia cell, such as a cell of the commercially available and/or fully characterized strains Ralstonia eutropha H16 and Ralstonia eutropha WP134. In another embodiment, the bacterial cell is a Corynebacterium cell, such as a cell of the commercially available and/or fully characterized strains 534 (ATCC 13032), K051, MB001, R, SCgG1, and SCgG2.

While aspect and embodiments relating to bacterial cells herein typically refer to genes or proteins according to their designation in E. coli, for bacterial cells of another family or species, it is within the level of skill in the art to identify the corresponding gene or protein, i.e., the ortholog and/or paralog, in the other family or species, typically by identifying sequences having moderate or high homology to the E. coli sequence, optionally taking the function of the protein expressed by the gene and/or the locus of the gene in the genome into account. Table 1 below sets out the function of the protein encoded by each specific gene, the corresponding E.C. number (if applicable), its locus in the E. coli K-12 MG1655 genome and the SEQ ID number of the coding sequence.

Table 2 below sets out some examples of homologs in selected organisms, identified in a preliminary and non-limiting analysis. Indeed, homologs of these proteins exist also in other bacteria, and other homologs not identified in this preliminary search can exist in the species listed in Table 2. The skilled person is well-familiar with different searching and/or screening methods for identifying homologs across different species.

TABLE 1
Protein function and Locus IDs

| E. coli | | E.C. | Locus | SEQ |
|---|---|---|---|---|
| designation | Protein function | number | ID | ID NO: |
| proV | ATP-binding subunit of glycine | 3.6.3.32 | b2677 | 1 |
| | betaine/proline ABC transporter | | | |
| proW | Membrane subunit of glycine | 3.6.3.32 | b2678 | 2 |
| | betaine/proline ABC transporter | | | |
| proX | Periplasmic binding protein | 3.6.3.32 | b2679 | 3 |
| | subunit of glycine | | | |
| | betaine/proline ABC transporter | | | |
| cspC | Stress protein, member of the | N/A | b1823 | 4 |
| | CspA family | | | |
| ptsP | Phosphoenolpyruvate-protein | 2.7.3.9 | b2829 | 5 |
| | phosphotransferase PtsP, | | | |
| | enzyme I^{Ntr} | | | |
| wbbK | Predicted lipopolysaccharide | N/A | b2032 | 6 |
| | biosynthesis protein | | | |
| yobF | Small protein involved in stress | N/A | b1824 | 7 |
| | responses | | | |
| nagC | NagC DNA-binding | N/A | b0676 | 8 |
| | transcriptional dual regulator | | | |
| nagA | N-acetylglucosamine-6- | 3.5.1.25 | b0677 | 9 |
| | phosphate deacetylase | | | |
| Rph | RNase PH | 2.7.7.56 | b3643 | 10 |
| yicC | Conserved protein | N/A | b3644 | 42 |
| yjcF | Conserved protein | N/A | b4066 | 43 |
| iscR | IscR DNA-binding transcriptional | N/A | b2531 | 44 |
| | dual regulator | | | |
| yedP | Predicted phosphatase | N/A | b1955 | 45 |
| ybeX | Putative transport protein | N/A | b0658 | 11 |
| Mpl | UDP-N-acetylmuramate: L- | 6.3.2.45 | b4233 | 12 |
| | alanyl-gamma-D-glutamyl- | | | |
| | meso-diaminoheptanedioate-D- | | | |
| | alanine ligase [multifunctional] | | | |
| ygaC | Predicted protein | N/A | b2671 | 13 (DNA) |
| | | | | 14 (protein) |
| rpsG | 30S ribosomal subunit protein | N/A | b3341 | 15 (DNA) |
| | S7 | | | 16 (protein) |
| argG | Argininosuccinate synthase | 6.3.4.5 | b3172 | 17 (DNA) |
| | | | | 18 (protein) |
| mreB | Cell wall structural actin-like | N/A | b3251 | 19 (DNA) |
| | protein in MreBCD complex; | | | 20 (protein) |
| | mecillinam resistance protein | | | |
| sspA | Stringent starvation protein A | N/A | b3229 | 21 (DNA) |
| | | | | 22 (protein) |
| nusA | transcription | N/A | b3169 | 23 (DNA) |
| | termination/antitermination L | | | 24 (protein) |
| | factor | | | |
| mrdB | Rod shape-determining | N/A | b0634 | 25 (DNA) |
| | membrane protein; sensitivity to | | | 26 (protein) |
| | radiation and drugs | | | |
| rpoD | RNA polymerase, sigma 70 | N/A | b3067 | 27 (DNA) |
| | (sigma D) factor | | | 28 (protein) |
| rpoC | RNA polymerase, β′ subunit | 2.7.7.6 | b3988 | 29 (DNA) |
| | | | | 30 (protein) |
| rpoB | RNA polymerase, β subunit | 2.7.7.6 | b3987 | 31 (DNA) |
| | | | | 32 (protein) |
| murA | UDP-N-acetylglucosamine | 2.5.1.7 | b3189 | 33 (DNA) |
| | enolpyruvyl transferase | | | 34 (protein) |
| rpsA | 30S ribosomal subunit protein | N/A | b0911 | 35 (DNA) |
| | S1 | | | 36 (protein) |
| spoT | Guanosine 3′-diphosphate 5′- | 3.1.7.2 | b3650 | 37 (DNA) |
| | triphosphate 3′-diphosphatase | | | 38 (protein) |
| | [multifunctional] | | | |
| pyrE | Orotate | 2.4.2.10 | b3642 | 39 (DNA) |
| | phosphoribosyltransferase | | | 40 (protein) |
| pyrE/rph | — | — | — | 41 |
| intergenic | | | | |
| region | | | | |

Table 2A and 28. Homologs or orthologs identified by protein BLAST (BLASTP) of E. coli K-12 MG1655 proteins against protein databases from selected reference organisms. Hits with the largest e-value are shown, and hits are only shown when the e-value <1.0. Hit proteins with e-value <0.1 (non-italicized) are deemed the most probable of having the same or similar function as the E. coli protein.

TABLE 2A

| Protein | | |
|---|---|---|
| (# of | | |
| residues) | B. subtilis 168 | P. putida KT2440 |
| ProV (400 | 51% identity (390 aa) | 51% identity (291 aa) “glycine |
| aa) | “glycine/betaine ABC transporter | betaine/L-proline ABC transporter ATP- |
| | ATP-binding protein” | binding subunit” (NP_742461.1); 51% |
| | (NP_388180.2); 37% identity (360 | identity (222 aa) “glycine betaine/L- |
| | aa) “glycine | proline ABC transporter |
| | betaine/carnitine/choline ATP- | ATPase/permease fusion protein” |
| | binding protein OpuCA” | (NP_744918.1); 36% identity (352 aa) |
| | (NP_391263.1); 36% identity (352 | “glycine betaine/L-proline ABC |
| | aa) “choline transport ATP-binding | transporter ATPase” (NP_743029.1) |
| | protein OpuBA” (NP_391253.1) | |
| ProW | 48% identity (275 aa) “glycine | 40% identity (265 aa) “glycine |
| (354 aa) | betaine transport system permease | betaine/L-proline ABC transporter |
| | protein OpuAB” (NP_388181.1); | permease” (NP_742462.1); 53% |
| | 31% identity (186 aa) “choline | identity (206 aa) “binding-protein- |
| | transport system permease protein | dependent transport system inner |
| | OpuBB” (NP_391252.1); 30% | membrane protein” (NP_745696.1); |
| | identity (187 aa) “glycine | 42% identity (259 aa) “glycine |
| | betaine/carnitine/choline transport | betaine/L-proline ABC transporter |
| | system permease protein OpuCB” | ATPase/permease fusion protein” |
| | (NP_391262.1); 30% identity (187 | (NP_744918.1) |
| | aa) “glycine | |
| | betaine/carnitine/choline transport | |
| | system permease protein OpuCD” | |
| | (NP_391260.1) | |
| ProX (330 | 25% identity (124 aa) “glycine | 27% identity (155 aa) “glycine betaine |
| aa) | betaine-binding protein OpuAC” | ABC transporter substrate-binding |
| | (NP_388182.1) | protein” (NP_745695.1); 24% identity |
| | | (200 aa) “glycine/betaine ABC |
| | | transporter substrate-binding protein |
| | | (NP_744919.1); 21% identity (327 aa) |
| | | “glycine betaine-binding protein” |
| | | (NP_742246.1) |
| CspC (70 | 67% identity (64 aa) “cold shock | 58% identity (64 aa) “cold shock |
| aa) | protein CspB” (NP_388791.1); 59% | protein CspA” (NP_744611.1); 60% |
| | identity (64 aa) “cold shock protein | identity (67 aa) “cold-shock domain- |
| | CspD” (NP_390076.1); 60% identity | contain protein” (NP_743146.1); 63% |
| | (62 aa) “cold shock protein CspC” | identity (61 aa) “cold-shock domain- |
| | (NP_388393.1) | contain protein” (NP_743260.1); 50% |
| | | identity (68 aa) “cold shock protein |
| | | CspA” (NP_743679.1); 55% identity |
| | | (64 aa) “cold-shock domain-contain |
| | | protein, partial” (NP_743369.1); 54% |
| | | identity (61 aa) “cold-shock protein |
| | | CspD” (NP_746140.1) |
| PtsP (749 | 33% identity (572 aa) | 43% identity (729 aa) “protein PtsP” |
| aa) | “phosphoenolpyruvate-protein | (NP_747246.1); 38% identity (575 aa) |
| | phosphotransferase” (NP_389274.2) | “phosphoenolpyruvate-protein |
| | | phosphotransferase” (NP_742954.1) |
| WbbK | 26% identity (110 aa) | 28% identity (111 aa) “glycosyl |
| (373 aa) | “glycosyltransferase YpjH” | transferase WbpY” (NP_743956.1) |
| | (NP_390127.1) | |
| YobF (48 | — | 42% identity (31 aa) “preprotein |
| aa) | | translocase subunit SecD” |
| | | (NP_742996.1) |
| NagC | 25% identity (358 aa) | — |
| (407 aa) | “transcriptional regulator” | |
| | (NP_389641.2) | |
| NagA | 32% identity (380 aa) “N- | 22% identity (391 aa) “guanine |
| (383 aa) | acetylglucosamine-6-phosphate | deaminase” (NP_746397.1) |
| | deacetylase” (NP_391381.1) | |
| Rph (228 | 58% identity (222 aa) “ribonuclease | 69% identity (228 aa) “ribonuclease |
| aa) | PH” (NP_390715.1) | PH” (NP_747395.1) |
| PyrE (213 | 25-34% identity (in stretches) | 67% identity (213 aa) “orotate |
| aa) | “orotate phosphoribosyl-transferase” | phosphoribosyl-transferase” |
| | (NP_389439.1) | (NP_747392.1) |
| YbeX (293 | 33% identity (253 aa) “hypothetical | 53% identity (268 aa) “hypothetical |
| aa) | protein BSU31300” (NP_391008.2); | protein PP_4789” (NP_746894.1) |
| | 32% identity (260 aa) “hypothetical | |
| | protein BSU24750” (NP_390355.1); | |
| | 33% identity (252 aa) “hypothetical | |
| | protein BSU26610” (NP_390538.1); | |
| | 33% identity (257 aa) “hypothetical | |
| | protein BSU09590” (NP_388840.1); | |
| | 29% identity (279 aa) “hypothetical | |
| | protein BSU9550” (NP_388836.1) | |
| Mpl (458 | 30% identity (386 aa) “UDP-N- | 59% identity (449 aa) “UDP-N- |
| aa) | acetylmuramate-L-alanine ligase” | acetylmuramate” (NP_742710.1); 29% |
| | (NP_390857.1) | identity (469 aa) “UDP-N- |
| | | acetylmuramate-L-alanine ligase |
| | | (NP_743497.1) |
| YgaC (115 | 29% identity (55 aa) | 29% identity (51 aa) “penicillin |
| aa) | “transglycosylase YomI” | amidase” (NP_{—}745045.1) |
| | (NP_{—}390018.2) | |
| RpsG | 56% identity (156 aa) “30S | 71% identity (156 aa) “30S ribosomal |
| (180 aa) | ribosomal protein S7” | protein S7” (NP_742616.1) |
| | (NP_387992.2) | |
| ArgG (448 | 29% identity (414 aa) | 26% identity (409 aa) “arginosuccinate |
| aa) | “arginosuccinate synthase” | synthase” (NP_743249.1) |
| | (NP_390823.1) | |
| MreB (348 | 58% identity (337 aa) “rod shape- | 80% identity (347 aa) “rod shape- |
| aa) | determining protein MreB” | determining protein MreB” |
| | (NP_390681.2) | (NP_743094.2) |
| SspA (212 | — | 57% identity (200 aa) “stringent |
| aa) | | starvation protein A” (NP_743480.1) |
| NusA (496 | 40% identity (343 aa) “transcription | 64% identity (493 aa) “transcription |
| aa) | termination/antitermination protein | elongation factor NusA” (NP_746821.1) |
| | NusA” (NP_389542.1) | |
| MrdB (371 | 32% identity (344 aa) “stage V | 53% identity (364 aa) “rod shape- |
| aa) | sporulation protein E” | determining protein RodA” |
| | (NP_389404.1); 30% identity (352 | (NP_746911.1) |
| | aa) “lipid II flippase FtsW” | |
| | (NP_389368.1); 30% identity (289 | |
| | aa) “rod shape-determining protein | |
| | RodA” (NP_391691.1) | |
| RpoD | 69% identity (238 aa) “RNA | 67% identity (610 aa) “RNA |
| (614 aa) | polymerase sigma factor RpoD” | polymerase sigma factor RpoD” |
| | (NP_390399.2) | (NP_742554.1) |
| RpoC | 50% identity (1134 aa) “DNA- | 75% identity (1399 aa) “DNA-directed |
| (1407 aa) | directed RNA polymerase subunit | RNA polymerase subunit beta′” |
| | beta′” | (NP_742614.1) |
| RpoB | 59% identity (533 aa) “DNA-directed | 72% identity (1360 aa) “DNA-directed |
| (1342 aa) | RNA polymerase subunit beta” | RNA polymerase subunit beta” |
| | | (NP_742613.1) |
| MurA (420 | 50% identity (422 aa) “UDP-N- | 61% identity (421 aa) “UDP-N- |
| aa) | acetylglucosamine-1 | acetylglucosamine 1- |
| | carboxyvinyltransferase 1” | carboxyvinyltransferase” |
| | (NP_391557.1); 45% identity (418 | (NP_743125.1) |
| | aa) “UDP-N-acetylglucosamine-1 | |
| | carboxyvinyltransferase 2” | |
| | (NP_391591.2) | |
| RpsA (558 | 39% identity (338 aa) “30S | 74% identity (554 aa) “30S ribosomal |
| aa) | ribosomal protein S1 homolog” | protein S1” (NP_743928.2) |
| | (NP_390169.1) | |
| SpoT (702 | 40% identity (719 aa) “GTP | 55% identity (701 aa) “(p)ppGpp |
| aa) | pyrophosphokinase” (NP_390638.2) | synthetase I SpoT/RelA” |
| | | (NP_747403.1); 37% identity (681 aa) |
| | | “(p)ppGpp synthetase I SpoT/RelA” |
| | | (NP_743813.1) |

TABLE 2B

| Protein | | |
|---|---|---|
| (# of | | Corynebacterium glutamicum |
| residues) | Ralstonia eutropha H16 | ATCC 13032 |
| ProV (400 | 40% identity (214 aa) “ABC | 34-40% identity (194-283 aa) “ABC |
| aa) | transporter ATPase” (YP_724876.1); | transporter ATPase” or “ABC |
| | 37% identity (239 aa) “ABC | transporter duplicated ATPase” |
| | transporter ATPase” (YP_726702.1); | (NP_599870.1, NP_601662.1, |
| | 33% identity (354 aa) “ABC | NP_599673.1, NP_599959.1, |
| | transporter ATPase” (YP_725457.1); | NP_601157.1, NP_600605.1, |
| | 39% identity (263 aa) “ABC | NP_600550.1, NP_601199.1, |
| | transporter ATPase” (YP_725326.1); | NP_602190.1, NP_599469.1, |
| | 39% identity (198 aa) “ABC | NP_601634.1, NP_601634.1, |
| | transporter ATPase” (YP_726845.1); | NP_601523.1, NP_601854.1, |
| | 39% identity (223 aa) “ABC | NP_600446.1, NP_600085.1, |
| | transporter ATPase” (YP_724565.1); | NP_600031.1, NP_600677.1) |
| | 38% identity (232 aa) “ABC | |
| | transporter ATPase” (YP_727745.1) | |
| ProW | 34% identity (194 aa) “ABC | 25-32% identity (124-232 aa) “ABC |
| (354 aa) | transporter permease” | transporter permease” |
| | (YP_725456.1); 32% identity (177 | (NP_600676.1, NP_600445.1, |
| | aa) “ABC-type transporter, fused | NP_601771.1, NP_599955.1) |
| | periplasmic and permease | |
| | components” (YP_726088.1); 31% | |
| | identity (152 aa) “ABC transporter | |
| | permease” (YP_725454.1); 29% | |
| | identity (175 aa) “ABC transporter | |
| | permease” (YP_726844.1) | |
| ProX (330 | 29% identity (75 aa) “RND | — |
| aa) | superfamily exporter” (YP_725351.1) | |
| CspC (70 | 61% identity (61 aa) “cold shock | 66% identity (65 aa) “cold shock |
| aa) | protein, DNA-binding” (YP_727497.1) | protein” (NP_599560.1); 62% |
| | | identity (65 aa) “cold shock protein” |
| | | (NP_599426.1); 34% identity (64 |
| | | aa) “cold shock protein” |
| | | (NP_600049.1) |
| PtsP (749 | 35% identity (497 aa) | 31% identity (539 aa) |
| aa) | “phosphoenolpyruvate-protein kinase | “phosphoenolpyruvate-protein |
| | (PTS system EI component)” | kinase” (NP_601139.1) |
| | (YP_724845.1); 32% identity (567 | |
| | aa) “protein-N(pi)-phosphohistidine- | |
| | sugar phosphotransferase II ABC” | |
| | (YP_724830.1) | |
| WbbK | 31% identity (103 aa) | 29% identity (75 aa) “group 1 |
| (373 aa) | “glycosyltransferase group 1” | glycosyltransferase” (NP_599714.1); |
| | (YP_726331.1); 25% identity (142 | 24% identity (139 aa) |
| | aa) “glycosyltransferase” | “glycosyltransferase” (NP_601390.1) |
| | (YP_727345.1) | |
| YobF (48 | — | — |
| aa) | | |
| NagC | 35% identity (48 aa) “ArsR family | 29% identity (273 aa) “glucose |
| (407 aa) | transcriptional regulator” | kinase” (NP_601389.1); 22% |
| | (YP_726640.1) | identity (186 aa) “transcriptional |
| | | regulator” (NP_601847.1); 21% |
| | | identity (327 aa) “transcriptional |
| | | regulator” (NP_599261.2) |
| NagA | 33% identity (330 aa) “N- | 24% identity (345 aa) “N- |
| (383 aa) | acetylglucosamine-6-phosphate | acetylglucosamine-6-phosphate |
| | deacetylase” (YP_724833.1) | deacetylase” (NP_601845.2) |
| Rph (228 | 62% identity (221 aa) “ribonuclease | 59% identity (217 aa) “ribonuclease |
| aa) | PH” (YP_725462.1) | PH” (NP_601703.2) |
| PyrE (213 | 56% identity (215 aa) “orotate | 29% identity (139 aa) “orotate |
| aa) | phosphoribosyl-transferase” | phosphoribosyl-transferase” |
| | (YP_724744.1) | (NP_601967.1) |
| YbeX (293 | 45% identity (259 aa) “Mg2+/Co2+ | 34% identity (259 aa) “hypothetical |
| aa) | transporter” (YP_725043.1); 26% | protein NCgl2206” (NP_601486.1); |
| | identity (269 aa) “Mg2+/Co2+ | 32% identity (225 aa) “hypothetical |
| | transporter” (YP_725283.1) | protein NCgl1393” (NP_600666.1); |
| | | 27% identity (307 aa) “hypothetical |
| | | protein NCgl1147” (NP_600420.1) |
| Mpl (458 | 60% identity (463 aa) “UDP-N- | 28% identity (484 aa) “UDP-N- |
| aa) | acetylmuramate-L-alanine ligase” | acetylmuramate-L-alanine ligase” |
| | (YP_727610.1); 28% identity (476 | (NP_601359.1) |
| | aa) “UDP-N-acetylmuramate-L- | |
| | alanine ligase” (YP_727714.1) | |
| YgaC (115 | 32% identity (37 aa) “glutathione S- | 32% identity (99 aa) “N-acetyl- |
| aa) | transferase” (YP_{—}727111.1) | gamma-glutamyl-phosphate |
| | | reductase” (NP_{—}600613.1) |
| RpsG | 65% identity (156 aa) “30S ribosomal | 57% identity (148 aa) “30S |
| (180 aa) | protein S7” (YP_727929.1) | ribosomal protein S7” |
| | | (NP_599739.1) |
| ArgG (448 | 33% identity (61 aa) “PP-loop | 29% identity (398 aa) |
| aa) | superfamily ATPase” (YP_727271.1) | “arginosuccinate synthase” |
| | | (NP_600619.1) |
| MreB (348 | 72% identity (350 aa) “rod shape- | 27% identity (220 aa) “molecular |
| aa) | determining protein MreB” | chaperone DnaK” (NP_601992.1) |
| | (YP_724633.1) | |
| SspA (212 | 46% identity (203 aa) “stringent | 56% identity (16 aa) “hypothetical |
| aa) | starvation protein A” (YP_727831.1) | protein NCgl2333” (NP_{—}601617.1) |
| NusA (496 | 50% identity (494 aa) “transcription | 32% identity (352 aa) |
| aa) | elongation factor NusA” | “transcriptional elongation factor |
| | (YP_726771.1) | NusA” (NP_601193.1) |
| MrdB (371 | 49% identity (364 aa) “rod-shape- | 30% identity (359 aa) “cell division |
| aa) | determining protein RodA” | membrane protein” (NP_601361.1); |
| | (YP_724637.1) | 28% identity (295 aa) “cell division |
| | | membrane protein” (NP_599296.1) |
| RpoD | 56% identity (618 aa) “RNA | 60% identity (241 aa) “RNA |
| (614 aa) | polymerase sigma factor RpoD” | polymerase sigma factor” |
| | (YP_727172.1); 47% identity (632 | (NP_601117.2) |
| | aa) “DNA-directed RNA polymerase | |
| | subunit (RpoD)” (YP_726126.1) | |
| RpoC | 67% identity (1397 aa) “DNA-directed | 50% identity (819 aa) “DNA- |
| (1407 aa) | RNA polymerase subunit beta′” | directed RNA polymerase subunit |
| | (YP_727932.1) | beta′” (NP_599734.1) |
| RpoB | 66% identity (1370 aa) “DNA-directed | 56% identity (616 aa) “DNA- |
| (1342 aa) | RNA polymerase subunit beta” | directed RNA polymerase subunit |
| | (YP_727933.1) | beta” (NP_599733.1) |
| MurA (420 | 60% identity (417 aa) “UDP-N- | 46% identity (417 aa) “UDP-N- |
| aa) | acetylglucosamine 1- | acetylglucosamine 1- |
| | carboxyvinyltransferase” | carboxyvinyltransferase” |
| | (YP_727854.1) | (NP_601757.1); 31% identity (425 |
| | | aa) “UDP-N-acetylglucosamine |
| | | enoylpyruvyl transferase” |
| | | (NP_599603.1) |
| RpsA (558 | 67% identity (529 aa) “30S ribosomal | 46% identity (340 aa) “30S |
| aa) | protein S1” (YP_725313.1) | ribosomal protein S1” |
| | | (NP_600575.1) |
| SpoT (702 | 47% identity (720 aa) “GTP | 38% identity (723 aa) “guanosine |
| aa) | pyrophosphokinase” (YP_725468.1); | polyphosphate pyrophospho- |
| | 36% identity (674 aa) “GTP | hydrolase/synthetase” |
| | pyrophosphokinase” (YP_725845.1) | (NP_600866.1); 33% identity (129 |
| | | aa) “guanosine polyphosphate |
| | | pyrophosphohydrolase/synthetase” |
| | | (NP_600534.2) |

So, in one aspect, there is provided a bacterial cell according to any one of the preceding aspects and embodiments, wherein each recited gene is instead (i) a gene encoding the corresponding protein in the Table above, (ii) a gene located at the corresponding locus, or (iii) both.

In particular, without being limited to theory, improved tolerance toward polyamines can be achieved by genetic modifications which
reduce the transporter-mediated import of polyamines, e.g., via the glycine betaine/proline ABC transporter or YbeX predicted transporter;
reduce cellular stresses imposed by a high level of extracellular binding of polyamines to the cell surface, via modifications to outer membrane saccharides (e.g. via the lipopolysaccharide biosynthesis protein WbbK) or modifications to cell shape that increase the surface area to volume ratio (e.g. via mutations in MreB, MurA, or MrdB)
modulate the NagC regulon either directly through a reduction in levels of NagC, or indirectly through reduced levels of NagA (which deacetylates N-acetyl-D-glucosamine 6-phosphate, a molecule which binds to NagC and causes dissociation from DNA);
restore improved expression of orotate phosphoribosyltransferase (PyrE), e.g. by deletion of rph;
improve general stress resistance toward stresses imposed by polyamines, e.g., by reduced levels of CspC and/or YobF; and/or
alter processes of cell wall recycling, e.g. by reduced levels of Mpl, NagC, or NagA.
reduce the effect of polyamines on the nucleoid by restoring levels of nucleoid-associated proteins such as H-NS and/or StpA, via mutations in SspA or YgaC.
reduce the effect of polyamines on altered transcription termination by mutations that reduce the activity of the NusA.
reduce the effects of excessive polyamine bound to ribosomes via mutations in RpsG and RpsA
reduce the efflux of Mg²⁺ cations or other divalent cations, which compete for nucleic acid binding with polyamines, e.g. via elimination of the Mg²⁺/divalent cation efflux transporter YbeX
reduce the intracellular levels of polyamines, e.g. by alteration of transport protein levels due to alterations in cell shape, alterations in the cell wall, and reduced levels of cation efflux transporters that would otherwise balance cellular charge with imported polyamines, e.g. via mutations in MreB, MurA, MrdB, Mpl, NagC, NagA, or YbeX.

So, in one embodiment, the bacterial cell has a genetic modification which reduces the expression of one or more endogenous proteins selected from the group consisting of
an ATP-binding subunit of glycine betaine/proline ABC transporter
a membrane subunit of glycine betaine/proline ABC transporter
a periplasmic binding protein subunit of glycine betaine/proline ABC transporter
a stress-protein member of the CspA family
a phosphoenolpyruvate-protein phosphotransferase PtsP, enzyme INtr
a lipopolysaccharide biosynthesis protein
a small protein involved in stress responses
a NagC DNA-binding transcriptional dual regulator
a N-acetylglucosamine-6-phosphate deacetylase
an RNase PH
a UDP-N-acetylmuramate:L-alanyl-gamma-D-glutamyl-meso-diaminoheptanedioate-D-alanine ligase
a transport protein involved in Mg²⁺/Co²⁺ or other divalent cation efflux

In addition, without being limited to theory, improved tolerance toward polyamine can also be achieved by genetic modifications which
reduce ribosomal frameshifting during translation of proteins under stress conditions imposed by polyamines, e.g., via a genetic modification in rpsG;
reduce levels of one or more precursors for intracellular biosynthesis of polyamines, e.g., via a genetic modification in argG or ygaC;
alter the cytoskeletal scaffold to increase the surface to volume ratio of the cell, e.g. via a genetic modification in mreB, mrdB, or murA;
reducing the effect of polyamines on altered transcription termination induced by high concentrations of polyamines, e.g., via a genetic modification in nusA; and/or
reduce the effect of polyamines on the nucleoid by restoring levels of nucleoid-associated proteins such as H—NS and/or StpA, via genetic modifications in sspA or ygaC.
reduce cellular stresses imposed by a high level of extracellular binding of polyamines to the cell surface, via modifications to cell shape that increase the surface area to volume ratio, e.g. via genetic modifications in mreB, mrdA or murA.
reduce intracellular levels of polyamines via reduced alteration of transport protein levels due to alterations in cell shape or alterations in the cell wall, e.g. via mutations in mreB, murA, mrdB, or mpl.
reduce the effects of excessive polyamine bound to ribosomes, e.g. via genetic modifications in rpsG and rpsA

In one specific embodiment, the bacterial cell further comprises a recombinant biosynthetic pathway for producing a polyamine, such as, e.g., putrescine, HMDA, spermidine, agmatine, 1,3-diaminopropane, cadaverine, ethylenediamine, citrulline and/or ornithine. In one additional embodiment, the bacterial cell is of the Corynebacterium genera. In one additional embodiment, the bacterial cell is of the Escherichia genera. In one additional embodiment, the bacterial cell is of the Bacillus genera. In one additional embodiment, the bacterial cell is of the Ralstonia genera. In one additional embodiment, the bacterial cell is of the Pseudomonas genera.

### Example 1

Methods

1) Screening for Tolerance in Wild-Type Cells

Escherichia coli K-12 MG1655 was grown overnight in M9 minimal medium+1% glucose and subcultured the following morning to an initial OD600 of 0.05 in M9+1% glucose. Cells were grown to mid-exponential phase (OD600 0.7-1.0) and were back-diluted with fresh medium to an OD600 of 0.7. The diluted cells were used to inoculate M9+1% glucose containing varying concentrations of putrescine dihydrochloride or HMDA dihydrochloride, and growth was measured in FlowerPlates in a Biolector microbioreactor system (m2p-labs) at 37° C. with 1000 rpm shaking. The culture volume in each well was 1.4 mL.

2) Adaptive Laboratory Evolution of Tolerant Strains

Based on the screening results, E. coli K-12 MG1655 was grown overnight in M9 minimal medium and 150 μL was transferred the next day into 8 tubes containing 15 mL of M9+1% glucose+25 g/L putrescine on a Tecan Evo robotic platform custom-designed for performing adaptive laboratory evolutions (ALE). Cells were cultured on a 37° C. heat block with stirring by magnetic stir bars. Culture OD600 was monitored at times determined by a predictive custom script, and when the OD600 reached approximately 0.3, 150 μL of culture was inoculated into a new tube with the same media concentration. Instrument downtime would occasionally result in cells overgrowing to saturation or an OD600 greater than 0.3, and reinoculations were occasionally performed from cryogenic stocks of the population. When the growth rate was observed to substantially increase, the media concentration was changed. These concentration changes for putrescine were to 30 g/L and 38 g/L, while the changes for HMDA were to 30 g/L, 35 g/L, and 38 g/L. Approximately 100 μL of each population (8 per chemical) were plated on LB agar and incubated at 37° C. overnight.

3) Primary Screening of ALE Isolates

Five colonies from wild-type K-12 MG1655 and 10 individual colonies deriving from each population were inoculated into 300 μL M9+1% glucose in 96 well deepwell plates and incubated in a 300 rpm plate shaker at 37° C. The next day, cells were diluted 10× in M9+1% glucose and 30 μL was transferred into clear-bottomed 96 well half-deepwell plates (with rectangular wells) containing M9+1% glucose and M9+1% glucose+42.22 g/L putrescine or HMDA, such that the final concentration of putrescine or HMDA was 38 g/L. In addition, cryogenic glycerol stocks of the overnight culture were saved in a 96 well plate format. Half deepwell plates were incubated at 37° C. with 225 rpm shaking in a Growth Profiler (Enzyscreen), with optical scans of the plates taken at 15 minute intervals. Green pixel values integrated over a 1 mm diameter circular area in each well were converted to OD600 values using a previously determined calibration between OD600 and green pixel values. Resulting growth curves were visually inspected for isolates exhibiting the most robust or unique growth patterns within each population. In general, it was attempted to select three isolates per population for further analysis, however isolates from some populations exhibited poor growth and were not considered further.

4) Secondary Screening of ALE Isolates

Selected isolates from the primary screen were restruck onto LB agar from the cryogenic stock made from the overnight culture plate for the primary screen. Five K-12 MG1655 colonies and three individual colonies from each isolate were inoculated as biological replicates into a new 96 well deepwell plate containing 300 μL of M9+1% glucose, and grown overnight as for the primary screen. The next day, a cryogenic stock and half deepwell plates containing M9+1% glucose with or without putrescine or HMDA were inoculated using the plate of overnight cultures, and growth was measured as described for the primary screen. Resulting growth curves were visually inspected for isolates exhibiting robust and reproducible growth between replicates in high concentrations of putrescine or HMDA.

5) Re-Sequencing of ALE Isolates

A total of 20 isolates were selected from the secondary screen for whole-genome resequencing. An individual colony was taken from the LB agar plates prepared following the primary screen, inoculated into 2 mL LB, and grown overnight at 37° C. in a 250 rpm shaker. The following morning, 0.5 mL of cells were transferred to microcentrifuge tubes and centrifuged at 16000×g for 2 minutes. The supernatant was removed and pellets were stored at −20° C. until further processing. Genomic DNA was extracted from thawed cell pellets using a PureLink genomic DNA extraction kit, with further concentration and purification performed by ethanol precipitation. To generate libraries for sequencing, the Illumina TruSeq Nano kit was used according to the manufacturers' directions using an input quantity of 200 ng of genomic DNA from each isolate. Sequencing was performed on an Illumina MiSeq sequencer, with a minimum 20× average genomic coverage ensured for each isolate based on the number of reads. Fastq output files were analyzed for variants compared to the K-12 MG1655 reference genome (accession number NC_000913.3) using breseq.

6) Construction of Gene Knockouts

Probable important losses-of-function (Group 1) were determined by identifying genes across all isolates that harbored mutations, especially those occurring in multiple populations, and by the presence of at least one mutation that either generated a premature stop codon, a frameshift mutation, or the presence of an insertion element sequence within the gene. For those genes, the corresponding knockout strain from the Keio collection of single knockout mutants (where each gene is replaced with a cassette consisting of a kanamycin resistance gene flanked by FRT sites) was used as a donor strain for P1vir phage transduction. Briefly, the Keio strain was grown to early exponential phase in LB+5 mM CaCl₂) and 80 μL of a P1vir stock raised on K-12 MG1655 was added. After significant lysis was observed after 1.5 to 2 hours, the lysate was filter-sterilized to remove cells and stored at 4° C. Strain K-12 MG1655 was grown overnight in LB+5 mM CaCl₂) and 100 μL of the overnight culture was mixed with 100 μL of the P1vir lysate of the Keio collection mutant, and the mixture was incubated at 37° C. without shaking for 20 minutes. The entire mixture was then plated on LB agar containing 1.25 mM sodium pyrophosphate as a chelating agent and 25 μg/mL kanamycin. One colony was then restruck on LB+1.25 mM Na2P4O7+25 μg/mL kanamycin plate and analyzed for presence of the Keio cassette in place of the wild-type gene by colony PCR. When further knockouts were constructed in the same strain, the Keio cassette was flipped out to generate a scar sequence such that KanR marker could be recycled. This was performed by transforming with pCP20, which constitutively expresses a flippase recombinase, and plating cells on LB agar+100 μg/mL ampicillin and incubating at 30° C. The next day, one or more colonies were tested by colony PCR for loss of the Keio cassette, and successful mutants were then cured of pCP20 by elevated temperature curing at 40° C. Strains were verified to be cured of plasmid by plating on LB agar+100 μg/mL ampicillin and incubation at 30° C. P1vir transductions were then performed using these mutant strains as recipients.

7) Biolector Growth Screening of Evolved Isolates and Reconstructed Mutants

Biological triplicate cultures of each strain were grown to saturation overnight in 96 well deepwell plates containing 300 μL M9+1% glucose. The next day, cells were diluted 1:10 in deionized water in a clear 96 well plate and the OD600 was measured on a BioTek plate reader. 48 well FlowerPlates containing a final volume of 1.4 mL of M9+1% glucose (plus relevant chemical) were inoculated to OD600 0.03 (with plate reader pathlength, 200 μL volume) with the overnight culture and sealed with Breathseal film. Light backscatter intensity was monitored in a Biolector microbioreactor system at 37° C. with 1000 rpm shaking.

8) Keio Collection Screening for Group 1 (Loss-of-Function) Mutations

For primary screening, Keio collection mutants were inoculated directly from a cryogenic stock of the Keio collection into 300 μL LB medium containing 25 μg/mL kanamycin in 96 well deepwell plates and grown at 37° C. with 300 rpm shaking overnight. The Keio background strain, BW25113, was also inoculated into wells of this plate as a control. A cryogenic stock was made from each plate, and the cryogenic stock was replica plated into another 96 well deepwell plate containing 300 μL M9+1% glucose and grown overnight. The next day, cells were inoculated 1:100 into clear bottomed 96 well half-deepwell plates containing M9+1% glucose plus 32 g/L and 38 g/L putrescine or HMDA and cultivated in a Growth Profiler as previously described for screening of ALE isolates.

As a secondary screen, promising Keio collection mutants were struck on LB+25 μg/mL kanamycin from the cryogenic stock plate prepared during primary screening above and biological triplicate colonies were inoculated into a 96 well deepwell plate containing 300 μL M9+1% glucose. The next day, cells were inoculated into plates for cultivation on the Growth Profiler as described above.

9) Conjugation-Mediated Genome Shuffling

To assist in the identification of causative mutations in selected evolved isolates, a technique was employed by conjugating the wild-type background strain K-12 MG1655 with Hfr (“High frequency of recombination”) mutants of the evolved isolates. In order to generate the Hfr mutants of evolved isolates, conjugations were first performed between evolved isolates transformed with pBAD30 (confers ampicillin resistance) with strains CAG60452 and CAG60453 (obtained from Prof. Jeffrey Barrick, University of Texas at Austin) which are 2,6-diaminopimelic acid auxotrophs that harbor integrated F plasmids containing a spectinomycin resistance marker at genomic loci at opposing ends of the genome. Following conjugation, evolved isolates harboring the integrated F plasmid were obtained by plating on LB agar containing both spectinomycin and ampicillin. These strains were subsequently conjugated over 1-2 days with 140 rpm shaking at 37° C. with K-12 MG1655 harboring pACYCDuet-1 (confers chloramphenicol resistance). The resulting conjugation mixture was plated on M9 agar plates containing 25 μg/mL chloramphenicol plus 38 g/L putrescine or 38 g/L HMDA, depending on the evolved isolate employed, to isolate only the wild-type strain. Larger colonies appearing either independently or overlaid on a background of slower growing, likely wild-type cells were picked and restruck on new plates containing chloramphenicol and putrescine or HMDA. Individual isolates were tested for their growth phenotype in biological triplicates and selected isolates were whole-genome resequenced.

10) Multiplex Automated Genome Engineering (MAGE)

Genomic point mutants were generated using MAGE (REF), which involves multiple cycles of electroporation of cells expressing the β protein of λ Red recombinase with single stranded DNA oligonucleotides. The single-stranded oligonucleotides are believed to behave like Okazaki fragments during DNA replication, and their use enables a high enough efficiency of allelic replacement to preclude needing to select for cells that received the mutation.

In this work, K-12 MG1655 was transformed with pMA7SacB (manuscript in revision), a plasmid that harbors the β subunit of λ Red recombinase and Dam (which we have shown in the manuscript in revision to enable low off-target mutation rates and preclude the use of mutator strains as is usually done when performing MAGE) under control of an arabinose-inducible promoter, and SacB to enable removing the plasmid by sucrose counterselection following the identification of a desired mutant. K-12 MG1655/pMA7SacB was grown in 15 mL of LB medium plus 100 μg/mL ampicillin to mid-exponential phase at 37° C., induced for 10 minutes with 0.2% L-arabinose, chilled in an ice water bath, and washed and concentrated 3 times with autoclaved chilled MilliQ water in a typical electrocompetent cell preparation. 50 pmol of oligonucleotide was added to a 50 μL aliquot of cells in a 1 mm gap electroporation cuvette, and cells were electroporated at 1.8 kV. Cells were immediately recovered in 1 mL LB and the entire volume of cells was used to inoculate the next 15 mL LB culture. Cells were grown to mid-exponential phase and the remainder of the procedure repeated, and recovered cells following electroporation were outgrown overnight to allow full genome segregation. The following morning, cells were plated on LB medium.

Colonies appearing on LB medium were then screened for the presence of the desired introduced mutation. Colonies were resuspended in water for use as a template in a quantitative PCR (qPCR) with a HotStart Taq master mix containing SYBR Green. To achieve discrimination of a mutated base via the cycle threshold, both wild-type and mutant forward primers were designed and run as separate reactions with the same reverse primer binding approximately 80-100 bp downstream of the mutation. The mutant forward primer had the last base designed to be complementary to the mutated base and an additional mutation at the −3 position from the 3′ end of the primer such that primer binding would be maximally destabilized with the wild-type base. The wild-type primer typically had the −3 position from the 3′ end of the primer mutated to offer additional destabilization with the mutant base. This allowed discrimination of the desired mutant or wild-type base for each screened isolate by qualitatively observing a reversal in the fluorescence vs. cycle threshold curves by qPCR with the two primer sets. Individual isolates were verified to have the desired mutant sequence in the genome with no adjacent off-target mutations by Sanger sequencing.

11) Cross-Compound Tolerance Screening

96 well deepwell plates containing 300 μL of M9+1% glucose were inoculated directly from cryogenic stocks made from precultures for the secondary screening of ALE isolates and were grown overnight at 37° C. with 300 rpm shaking. The next day, cells were diluted 1:100 into 96 well half-deepwell plates containing the following final concentrations of each chemical in M9+1% glucose:

| | | |
|---|---|---|
| Butanol | 1.4% | v/v |
| Glutarate | 40 | g/L |
| p-coumarate | 7.5 | g/L |
| Putrescine | 32 | g/L |
| HMDA | 32 | g/L |
| Adipate | 45 | g/L |
| Isobutyrate | 7.5 | g/L |
| Hexanoate | 3 | g/L |
| Octanoate | 8 | g/L |
| 2,3-butanediol | 6% | v/v |
| 1,2-propanediol | 6% | v/v |

| | |
|---|---|
| sodium chloride | 0.6M |

Plates were cultivated in a Growth Profiler for 48 hours as described for screening of ALE isolates. Green pixel integrated values from each well were converted to OD600 values using a calibration curve and the resulting OD600 vs. elapsed time data was processed using custom scripts to determine the time required for each culture to reach an OD of 1.0 (tOD1). This value is a combined measure of growth rate and lag time in each culture. The median value was taken for biological triplicates of each isolate and was normalized to the median tOD1 for K-12 MG1655 controls (5 replicates). The ratio of tOD1(evolved)/tOD1(wild-type) is presented.

12) Flow Cytometry

In preliminary tests, overnight precultures of each strain picked from single colonies on LB plates were grown in M9+1% glucose to saturation overnight. Cells were diluted 1:100 directly into 100 μL phosphate buffered saline (PBS) and 1 μL of 10× diluted SYTOX Green was added. Flow cytometry was performed on a Fortessa flow cytometer (Becton Dickenson) with forward and side scatter channels set to 220 V. Events were thresholded with a minimum forward scatter value of 200.

In later screens of all strains, overnight cultures were replica plated from stored cryogenic plates containing all secondary screened PUTR isolates into 300 μL M9+1% glucose in 96 well deepwell plates. Cells were grown to saturation overnight and subcultured into new cultures containing M9 or M9+38 g/L putrescine (both plus 1% glucose). At different timepoints that represented exponential or stationary phase for the majority of strains in each condition, 100 μL of each culture was harvested, spun down, the media was removed, and resuspended in 100 μL of PBS. Resuspended cells were diluted 1:100 in PBS with SYTOX Green added as above, and cells were analyzed as described above.

13) Phase Contrast Microscopy

Cells were grown to exponential phase as described for flow cytometry. Glass slides were prepared with a thin layer of LB agar and a small volume of cell culture was spotted onto the agar and covered with a glass cover slip. Images were obtained under phase contrast on a Leica Microsystems fluorescence microscope with white light backlighting and 1000× total magnification with a 100× oil immersion lens.

14) Construction of Production Strains

Strain XQ52 and plasmid p15SpeC were generously provided by S. Y. Lee (Qian et al., 2009). For screening of putrescine production in ALE evolved isolates, p15SpeC was transformed into each isolate and K-12 MG1655 as a control. Briefly, cells were grown to exponential phase in LB medium, transferred to an ice water bath, centrifuged at 4000×g for 5 minutes in a refrigerated microcentrifuge, and the pellet was resuspended in 1/20th of the original culture volume of TSS buffer (5 g PEG 8000, 1.5 mL of 1 M MgCl2, 2.5 mL of DMSO brought up to 50 mL total volume with LB medium and filter-sterilized). Approximately 100 ng of plasmid DNA was added to the resuspended cells, and after approximately 10 minutes incubation in an ice-water bath, cells were heat shocked at 42° C. for 30 seconds and transferred to an ice water bath for 2 minutes. LB medium was added and cells were outgrown for ˜1 hour before plating on LB plates containing 50 μg/mL kanamycin. Mutations were additionally made in strain XQ52 by MAGE, as described previously, and p15SpeC was transformed as described above to generate an additional set of production strains.

15) Cell Culturing for Putrescine Production

For putrescine production under batch conditions, cells were inoculated directly from colonies on fresh transformation plates into 300 μL of LB medium containing 50 μg/mL kanamycin in 96 well deepwell plates, and grown in a plate shaker overnight at 37° C. with 300 rpm shaking. The following morning, cells were diluted 1:100 into a final volume of either 2.5 mL of R/2 medium (2 g/L (NH4)2HPO4, 6.75 g/L KH2PO4, 0.85 g/L citric acid, 0.7 g/L MgSO4.7H2O, 0.1 mM CaCl₂, trace elements as supplemented in M9 medium described previously, 10 g/L glucose, and 3 g/L (NH4)2SO4; pH adjusted to 6.80) containing 50 μg/mL kanamycin in 24 well MTP plates or 300 μL of R/2 medium in 96 well MTP plates. When strains were not lacI− (as in the XQ52 background), 100 μM isopropyl β-D-1-thiogalactopyranoside (IPTG) was added at inoculation to induce overexpression of SpeC on p15SpeC. Cultures were incubated in a plate shaker at 37° C. with 300 rpm shaking, and samples were taken after 24 h and/or 48 h for analysis of putrescine production. One-tenth volume of 100% trichloroacetic acid and a final volume of 0.5 g/L HMDA was added as an internal standard. After vortexing and centrifugation, supernatants were stored at −20□C before further processing for polyamine analysis.

For semi-batch growth with periodic glucose/ammonia feeding, cells were inoculated directly from colonies on fresh transformation plates into 2.5 mL of LB medium containing 50 μg/mL kanamycin in 24 well deepwell plates, and grown in a plate shaker overnight at 37° C. with 300 rpm shaking. The next morning, after withdrawing 100 μL for measuring OD600, the remaining culture volume was spun down in plates at 4000 rpm for 10 minutes, cell pellets were resuspended in 500 μL of R/2 medium (previously described), and cells were inoculated into 10.5 mL of R/2 medium containing 50 μg/mL kanamycin in Hamilton fermentors on a Hamilton Vantage™ based cultivation robot. IPTG was added to lacI+ strains to a final concentration of 100 μM. A feed solution containing 500 g/L glucose, 154.5 g/L (NH4)2SO4, and 7.27 g/L MgSO4.7H2O (filter-sterilized) was fed into the fermentors. After 24 and 48 hours cultivation, 0.5 mL of cell culture was collected, 10 μL of 100 g/L HMDA was added as an internal standard, and 50 μL of 100% (w/v) trichloroacetic acid was added. Following vortexing and centrifugation, supernatants were stored at −20° C. before further processing for polyamine analysis.

16) Derivatization and HPLC Analysis of Polyamines

50 μL of supernatants collected as described above were transferred to a glass tube containing 200 μL of 2 M NaOH. To this solution, 10 μL of 50% (v/v) benzoyl chloride in methanol was added to the tubes and they were immediately vortexed for 30 seconds to disperse the benzoyl chloride. The benzoylation reaction was allowed to proceed for 30 minutes, with vortexing approximately every 5 minutes. Benzoylated polyamines were then extracted into 1 mL of chloroform and 500 μL of the bottom chloroform layer was transferred to a new tube and evaporated to dryness under a nitrogen stream. To the dried residue in the tubes, 500 μL of 50% (v/v) acetonitrile in water was added. An external standard containing putrescine with 0.5 g/mL of HMDA as an internal standard was similarly prepared using the same procedure, and dilutions were made to enable the determination of a standard curve. 10 μL of each sample was injected on an Ultimate 3000 HPLC (Thermo Scientific) equipped with a Discovery® HS F5 column (2.1×150 mm, 3.0 μm particle size) (Supelco) with a UV detector (229 nm). The mobile phase consisted of 10 mM ammonium formate, pH 3 adjusted with formic acid (A) and acetonitrile (B), with the following linear gradients applied using a total flow rate of 0.5 mL/min: 10% B from 0 to 2 minutes, 10 to 45% B from 2 to 22 minutes, 45% B from 22 to 26 minutes, 45 to 10% B from 26 to 28 minutes, and 10% B from 28-30 minutes. Putrescine, cadaverine, HMDA, and excess benzoyl chloride appeared as peaks at retention times of 14.5, 15.9, 17.8, and 15.1 minutes, respectively.

17) Analysis of Growth Parameters (Growth Rate and Lag Time)

For data obtained with the Biolector microbioreactor system, self-baselined growth series were imported directly into a custom software platform that automatically detects growth phases and exports growth rates and lag times. In an earlier version of the software (values labeled in columns with “(1)”, a line was fit to a detected linear region in semilog space to determine the growth rate. An updated version of the software (values labeled in columns with “(2)”) implemented a direct exponential fit of a detected growth phase in linear space, resulting in higher weighting of the least squares fit to regions of the curve exhibiting higher growth. Additionally, the updated version of the software implemented an adaptive smoothing algorithm that split the data into variable sized windows that minimize the standard deviation of growth values within a time interval, and generated spline fits between points. Finally, the updated version of the software discarded regions where growth curves were fit but the signal-to-noise ratio was less than 1, to eliminate automatic detection of false growth phases. While automatic detection succeeded in detecting and fitting the dominant growth phase more than 95% of the time, all data was additionally manually curated to ensure that the main growth phase was always selected and that false growth phases were not detected when growth was essentially absent.

For data obtained with the Growth Profiler, improved image analysis was additionally implemented to obtain the updated growth parameters. In the Tables below, for values labeled in columns with “(1)”, integrated pixel values (which were later converted to OD₆₀₀ using a calibration curve) were obtained directly from image analysis capabilities in the Growth Profiler software. In the Tables below, for values labeled in columns with “(2)”, a new algorithm was implemented that automatically detected the pixel integration region in each well in each image by locating the darkest pixels in each well. These values were converted to OD₆₀₀ with a calibration run in the same manner. The new algorithm provided for an improved accuracy in determining the growth rate, since it eliminated a slowly oscillating frequency that was sometimes observed in the original data, potentially related to the practical setup when scanning the plates.

Results

a) Wild-Type Tolerance to Polyamines

The maximum measured concentration of putrescine at which K-12 MG1655 can grow was found to be 40 g/L (Table 3), with a nearly 26 hour lag time. Lag times and growth rates dropped steeply at concentrations above 30 g/L. At concentrations above 40 g/L, no growth was detected.

The maximum measured concentration of HMDA at which exponentially growing K-12 MG1655 can grow was found to be 40 g/L (Table 4). At concentrations above 40 g/L, there was a steep drop in growth, with zero growth detected at 50 g/L concentration.

TABLE 3
Growth of K-12 MG1655 in putrescine

| | mean (1) | | std. error (1) | | mean (2) | | std. error (2) | |
|---|---|---|---|---|---|---|---|---|
| putrescine | μ | t_{lag} | μ | t_{lag} | μ | t_{lag} | μ | t_{lag} |
| (g/L) | (h⁻¹) | (h) | (h⁻¹) | (h) | (h⁻¹) | (h) | (h⁻¹) | (h) |
| 0 | 0.926 | 1.3 | 0.178 | 0.1 | 0.651 | 0.5 | 0.026 | 0.2 |
| 10 | 0.698 | 1.4 | 0.086 | 0.0 | 0.538 | 1.0 | 0.015 | 0.1 |
| 20 | 0.577 | 2.2 | 0.020 | 0.2 | 0.392 | 1.5 | 0.011 | 0.1 |
| 30 | 0.350 | 3.4 | 0.057 | 0.7 | 0.317 | 6.2 | 0.007 | 0.3 |
| 40 | 0.108 | 12.3 | 0.041 | 3.0 | 0.148 | 25.8 | 0.016 | 5.8 |
| 50 | 0.023 | 19.8 | 0.040 | — | 0.000 | — | 0.000 | — |
| 75 | 0.000 | — | 0.000 | — | 0.000 | — | 0.000 | — |
| 100 | 0.000 | — | 0.000 | — | 0.000 | — | 0.000 | — |

TABLE 4
Growth of K-12 MG1655 in HMDA

| | mean (1) | | std. error (1) | | mean (2) | | std. error (2) | |
|---|---|---|---|---|---|---|---|---|
| HMDA | μ | t_{lag} | μ | t_{lag} | μ | t_{lag} | μ | t_{lag} |
| (g/L) | (h⁻¹) | (h) | (h⁻¹) | (h) | (h⁻¹) | (h) | (h⁻¹) | (h) |
| 0 | 0.946 | 1.4 | 0.032 | 0.0 | 0.698 | 0.8 | 0.005 | 0.1 |
| 10 | 0.806 | 1.3 | 0.059 | 0.1 | 0.605 | 0.8 | 0.021 | 0.1 |
| 20 | 0.601 | 1.4 | 0.047 | 0.1 | 0.394 | 0.3 | 0.005 | 0.1 |
| 30 | 0.475 | 1.7 | 0.056 | 0.2 | 0.241 | 0.0 | 0.005 | 0.0 |
| 40 | 0.216 | 2.6 | 0.026 | 0.2 | 0.136 | 2.3 | 0.003 | 0.7 |
| 50 | 0.000 | — | 0.000 | — | 0.000 | — | 0.000 | — |
| 75 | 0.000 | — | 0.000 | — | 0.000 | — | 0.000 | — |
| 100 | 0.000 | — | 0.000 | — | 0.000 | — | 0.000 | — |

Aiming for a starting growth rate of approximately 0.3 h-1, it was decided to begin evolutions at a concentration of 25 g/L putrescine and 25 g/L HMDA.

b) Resequencing of Tolerant Isolates

Variants detected in putrescine and HMDA evolved strains are presented in Tables 5 and 6, respectively. Each strain name corresponds to the chemical the strain was isolated from, the population the strain was isolated from, and the original number of the strain assigned during primary screening (e.g. PUTR3-1 is a putrescine-evolved strain isolated from population 3). In each table, strains are arranged such that all that were isolated from the same population are presented in the same rows. Strains with an asterisk (*) following their name are hypermutator strains, and only the mutation identified that can be associated with generating the hypermutator phenotype (here only in mutS or mutT in 2 HMDA populations) and those mutations that are shared with other mutations in the same gene in other strains are shown.

TABLE 5
Variants detected in putrecine-evolved isolates

| coordinate | gene | change | coordinate | gene | change | coordinate | gene | change |
|---|---|---|---|---|---|---|---|---|
| PUTR2-4 | | | PUTR2-6 | | | | | |
| 1907266 | cspC | 7 bp insertion | 1907266 | cspC | 7 bp insertion | | | |
| | | (→CGTCCTG) | | | (→CGTCCTG) | | | |
| 3400986 | mreB | N34K (A→C) | 3400986 | mreB | N34K (A→C) | | | |
| 4186706 | rpoC | V453I (G→A) | 4186706 | rpoC | V453I (G→A) | | | |
| PUTR3-1 | | | PUTR3-9 | | | PUTR3-10 | | |
| 1211308 | mcrA/icdC | noncoding SNP | 2678755 | yphF | IS5 element | 1211308 | mcrA/icdC | noncoding SNP |
| | | (G→T) | | | insertion | | | (G→T) |
| 1934806 | edd/zwf | noncoding SNP | 2774803 | yfjW | 1 bp deletion | 2661793 | iscR | L113F (C→A) |
| | | (C→T) | | | | | | |
| 2799867 | ygaC | R43L (C→A) | 3400453 | mreB | E212A (T→G) | 2799867 | ygaC | R43L (C→A) |
| 2804946 | proV | 1 bp insertion | 3816611 | rph/yicC | noncoding SNP | 2804946 | proV | 1 bp insertion |
| | | (→T) | | | (C→A) | | | (→T) |
| 3816611 | rph/yicC | noncoding SNP | 3823025 | spoT | R209H (G→A) | 2904286 | ygcE/queE | 122 bp deletion |
| | | (C→A) | | | | | | |
| 3823025 | spoT | R209H (G→A) | 3911364 | pstS | 1 bp insertion | 3816611 | rph/yicC | noncoding SNP |
| | | | | | (→T) | | | (C→A) |
| 4178239 | nusG | G166V (G→T) | 4257602 | lexA | N163I (A→T) | 3823025 | spoT | R209H (G→A) |
| 4257602 | lexA | N163I (A→T) | | | | 4178239 | nusG | G166V (G→T) |
| | | | | | | 4257602 | lexA | N163I (A→T) |
| | | | | | | 4267824 | tyrB | L237F (A→C) |
| PUTR4-3 | | | PUTR4-7 | | | PUTR4-8 | | |
| 457398 | clpP/dpX | 7 bp deletion | 665554 | mrdB | E254K (C→T) | 665554 | mrdB | E254K (C→T) |
| 665554 | mrdB | E254K (C→T) | 962473 | rpsA | D160V (A→T) | 962473 | rpsA | D160V (A→T) |
| 1907410 | cspC | IS5 element | 1907410 | cspC | IS5 element | 1236007 | ycgB | 50 bp deletion |
| | | insertion | | | insertion | | | |
| 2805131 | proV | 1 bp insertion | 2805131 | proV | 1 bp insertion | 1907410 | cspC | IS5 element |
| | | (→T) | | | (→T) | | | insertion |
| 4183154 | rpoB | R637L (G→T) | 4183154 | rpoB | R637L (G→T) | 2805131 | proV | 1 bp insertion |
| | | | | | | | | (→T) |
| | | | | | | 4183154 | rpoB | R637L (G→T) |
| | | | | | | 4392443 | glyV/glyX | 1 bp deletion |
| PUTR5-1 | | | PUTR5-6 | | | PUTR5-8 | | |
| 568660 | emrE/ybcK | noncoding SNP | 3214770 | rpoD | E575A (A→C) | 3214770 | rpoD | E575A (A→C) |
| | | (C→T) | | | | | | |
| 1755770 | pykF | D25N (G→A) | 4186551 | rpoC | V401G (T→G) | 4186551 | rpoC | V401G (T→G) |
| 2023551 | fliR | IS5 element | 4452005 | ytfR | noncoding SNP | | | |
| | | insertion | | | (G→A) | | | |
| 3401016 | mreB | I24M (A→C) | | | | | | |
| 3805049 | waaS | 1 bp deletion | | | | | | |
| 3823799 | spoT | R467L (G→T) | | | | | | |
| 3910569 | pstS | 7 bp deletion | | | | | | |
| 4522146 | yjhG | D650V (T→A) | | | | | | |
| PUTR6-2 | | | PUTR6-7 | | | PUTR6-10 | | |
| 2798597 | stpA/alaE | IS1 element | 576891 | nmpC/essD | noncoding SNP | 576891 | nmpC/essD | noncoding SNP |
| | | insertion | | | (C→T) | | | (C→T) |
| 3336073 | murA | G141A (C→G) | 962933 | rpsA | N313K (C→G) | 777151 | tolA | 48 bp deletion |
| 3377150 | sspA | V91F (C→A) | 1199680 | intE | IS1 element | 962933 | rpsA | N313K (C→G) |
| | | | | | insertion | | | |
| 3899249 | yieK | T49P (T→G) | 1879829 | yeaR | IS186 element | 1199680 | intE | IS1 element |
| | | | | | insertion | | | insertion |
| 3908805 | pstA | 2 bp deletion | 1907448 | yobF | IS5 element | 1879829 | yeaR | IS186 element |
| | | | | | insertion | | | insertion |
| | | | 2804858 | proV | 13 bp deletion | 1907448 | yobF | IS5 element |
| | | | | | | | | insertion |
| | | | 3079559 | cmtB/tktA | noncoding SNP | 2804858 | proV | 13 bp deletion |
| | | | | | (C→T) | | | |
| | | | 3197145 | glnE | 12 bp deletion | 3079559 | cmtB/tktA | noncoding SNP |
| | | | | | | | | (C→T) |
| | | | 3815859 | rph | 82 bp deletion | 3267294 | tdcA/tdcR | noncoding SNP |
| | | | | | | | | (G→T) |
| | | | 4186186 | rpoC | noncoding SNP | 3815859 | rph | 82 bp deletion |
| | | | | | (G→C) | | | |
| | | | | | | 4282760 | yjcF | R106G (T→C) |
| PUTR7-1 | | | PUTR7-7 | | | PUTR7-9 | | |
| 962922 | rpsA | D310Y (G→T) | 3214770 | rpoD | E575A (A→C) | 1673532 | mdtJ/tqsA | 181 bp deletion |
| 3316916 | nusA | M204R (A→C) | 3335317 | murA | Y393S (T→G) | 3214770 | rpoD | E575A (A→C) |
| 3400811 | mreB | H93N (G→T) | 4183154 | rpoB | R637L (G→T) | 3335317 | murA | Y393S (T→G) |
| 3823799 | spoT | R467H (G→A) | | | | 4183154 | rpoB | R637L (G→T) |
| PUTR8-3 | | | PUTR8-6 | | | PUTR8-10 | | |
| 2807247 | proX | 1 bp insertion | 83670 | leuL | 3 bp deletion | 562667 | sfmH | F11S (T→C) |
| | | (→T) | | | | | | |
| 3318960 | argG | noncoding SNP | 701231 | nagC | 1 bp deletion | 700785 | nagC | 47 bp deletion |
| | | (C→A) | | | | | | |
| 3400195 | mreB | A298V (G >A) | 2025435 | yodD/yedP | noncoding SNP | 2807247 | proX | 1 bp insertion |
| | | | | | (T→A) | | | (→T) |
| 3473612 | rpsG | L157* (A→C) | 2807247 | proX | 1 bp insertion | 3318960 | argG | noncoding SNP |
| | | | | | (→T) | | | (C→A) |
| 3815801 | pyrE/rph | 1 bp deletion | 3318960 | argG | noncoding SNP | 3400195 | mreB | A298V (G→A) |
| | | | | | (C→A) | | | |
| 3823811 | spoT | R471H (G→A) | 3400195 | mreB | A298V (G→A) | 3473612 | rpsG | L157* (A→C) |
| | | | 3473612 | rpsG | L157* (A→C) | 3815801 | pyrE/rph | 1 bp deletion |
| | | | 3815801 | pyrE/rph | 1 bp deletion | 3823811 | spoT | R471H (G→A) |
| | | | 3823811 | spoT | R471H (G→A) | | | |

TABLE 6
Variants detected in HMDA-evolved isolates

| coordinate | gene | change | coordinate | gene | change | coordinate | gene | change |
|---|---|---|---|---|---|---|---|---|
| HMDA1-10 | | | | | | | | |
| 962939 | rpsA | N315K (C→A) | | | | | | |
| 2694102 | purL | C481F (C→A) | | | | | | |
| 2804858 | proV | 13 bp deletion | | | | | | |
| 3816611 | rph/yicC | noncoding SNP | | | | | | |
| | | (C→A) | | | | | | |
| 3823025 | spoT | R209H (G→A) | | | | | | |
| 4181786 | rpoB | G181V (G→T) | | | | | | |
| 4257602 | lexA | N163I (A→T) | | | | | | |
| HMDA2-1 | | | HMDA2-8 | | | | | |
| 963273 | rpsA | 3 bp substitution | 963273 | rpsA | 3 bp substitution | | | |
| | | (→CGT) | | | (→CGT) | | | |
| 2804836 | proV | IS1 element | 2804836 | proV | IS1 element | | | |
| | | insertion | | | insertion | | | |
| 2968160 | ptsP | 1 bp deletion | 2968160 | ptsP | 1 bp deletion | | | |
| 3815801 | pyrE/rph | 1 bp deletion | 3815801 | pyrE/rph | 1 bp deletion | | | |
| HMDA3-4 | | | HMDA3-5 | | | HMDA3-6 | | |
| 702592 | nagA | 1 bp insertion | 691772 | ybeX | 12 bp deletion | 702592 | nagA | 1 bp insertion |
| | | (→G) | | | | | | (→G) |
| 2798606 | alaE [proW] | 7565 bp deletion | 701405 | nagC | E64* (C→A) | 2177307 | gatY/fbaB | IS1 element |
| | | | | | | | | insertion |
| 3815809 | pyrE/rph | 1 bp deletion | 2798606 | alaE [proW] | 7565 bp deletion | 2798606 | alaE [proW] | 7565 bp deletion |
| 3933122 | kup | P603T (C→A) | 2879763 | ygbT | L4F (G→A) | 3815809 | pyrE/rph | 1 bp deletion |
| 4188767 | rpoC | R1140C (C→T) | 2991218 | ygeF/ygeG | noncoding SNP | 3933122 | kup | P603T (C→A) |
| | | | | | (A→G) | | | |
| | | | 3815809 | pyrE/rph | 1 bp deletion | 4188767 | rpoC | R1140C (C→T) |
| | | | 3933122 | kup | P603T(C→A) | | | |
| HMDA4-2* | | | HMDA4-6* | | | HMDA4-9* | | |
| 962923 | rpsA | D310G (A→G) | 962923 | rpsA | D310G (A→G) | 962923 | rpsA | D310G (A→G) |
| 1729289 | lhr | R68Q (G→A) | 2805832 | proV | IS1 element | 2805832 | proV | IS1 element |
| | | | | | insertion | | | insertion |
| 2805832 | proV | IS1 element | 2859212 | mutS | 6 bp insertion | 2859212 | mutS | 6 bp insertion |
| | | insertion | | | (→GGCGTG) | | | (→GGCGTG) |
| 2859212 | mutS | 6 bp insertion | 3815859 | rph | 82 bp deletion | 3815859 | rph | 82 bp deletion |
| | | (→GGCGTG) | | | | | | |
| 3815859 | rph | 82 bp deletion | 3823861 | spoT | R488C (C→T) | 3823861 | spoT | R488C (C→T) |
| 3823861 | spoT | R488C (C→T) | 4185708 | rpoC | L120P (T→C) | 4185708 | rpoC | L120P (T→C) |
| 4181706 | rpoB | noncoding SNP | | | | | | |
| | | (C→T) | | | | | | |
| 4185708 | rpoC | L120P (T→C) | | | | | | |
| HMDA5-4 | | | HMDA5-5 | | | HMDA5-10 | | |
| 700602 | nagC | IS1 element | 691321 | ybeX | L155Q (A→T) | 2798597 | stpA/alaE | IS1 element |
| | | insertion | | | | | | insertion |
| 2798606 | alaE [proV] | 7067 bp deletion | 700602 | nagC | IS1 element | 2966573 | ptsP | D621A (T→G) |
| | | | | | insertion | | | |
| 2966573 | ptsP | D621A (T→G) | 2798606 | alaE [proV] | 7067 bp deletion | 3815809 | pyrE/rph | 1 bp deletion |
| 3310266 | pnp | E301D (C→A) | 2966573 | ptsP | D621A (T→G) | 3908248 | pstB | A40E (G→T) |
| 3815809 | pyrE/rph | 1 bp deletion | 3815809 | pyrE/rph | 1 bp deletion | 4485639 | pepA | S105A (A→C) |
| 4378331 | ampC | I205T (A→C) | | | | | | |
| HMDA6-3* | | | HMDA6-7* | | | | | |
| 111300 | mutT | L86P (T→C) | 111300 | mutT | L86P (T→C) | | | |
| 701839 | nagA | T305P (T→G) | 701839 | nagA | T305P (T→G) | | | |
| 2804831 | proV | 7 bp deletion | 2804831 | proV | 7 bp deletion | | | |
| 2879197 | ygbT | E192D (T→G) | 2879197 | ygbT | E192D (T→G) | | | |
| 3815859 | rph | 82 bp deletion | 3104042 | mutY | L344V (T→G) | | | |
| 3823987 | spoT | G530C (G→T) | 3815859 | rph | 82 bp deletion | | | |
| | | | 3823987 | spoT | G530C (G→T) | | | |
| HMDA7-1 | | | HMDA7-7 | | | HMDA7-10 | | |
| 2104070 | wbbK | 1 bp deletion | 3317072 | nusA | L152R (A→C) | 2104070 | wbbK | 1 bp deletion |
| 2818240 | argY/argV | 284 bp deletion | 3377173 | sspA | F83C (A→C) | 2818220 | argY/argV | 271 bp deletion |
| 3317072 | nusA | L152R (A→C) | 3473612 | rpsG | L157* (A→C) | 3317072 | nusA | L152R (A→C) |
| 3377173 | sspA | F83C (A→C) | | | | 3377173 | sspA | F83C (A→C) |
| 3473612 | rpsG | L157* (A→C) | | | | 3473612 | rpsG | L157* (A→C) |
| HMDA8-5 | | | HMDA8-9 | | | HMDA8-10 | | |
| 358399 | cynR | T98P (T→G) | 700980 | nagC | Y205* (G→C) | 700980 | nagC | Y205* (G→C) |
| 700980 | nagC | Y205* (G→C) | 1728512 | rnt | T56P (A→C) | 1728512 | rnt | T56P (A→C) |
| 1728512 | rnt | T56P (A→C) | 1732811 | lhr | S1242I (G→T) | 1732811 | lhr | S1242I (G→T) |
| 1732811 | lhr | S1242I (G→T) | 1744313 | mdtK | V286E (T→A) | 1744313 | mdtK | V286E (T→A) |
| 1744313 | mdtK | V286E (T→A) | 2804858 | proV | 13 bp deletion | 2804858 | proV | 13 bp deletion |
| 2522653 | xapR/xapB | noncoding SNP | 3815801 | pyrE/rph | 1 bp deletion | 3815801 | pyrE/rph | 1 bp deletion |
| | | (G→A) | | | | | | |
| 2804858 | proV | 13 bp deletion | | | | 4457112 | mpl | 4 bp deletion |
| 3815801 | pyrE/rph | 1 bp deletion | | | | | | |

c) Characterization of Selected Isolates

Each re-sequenced isolate was characterized using the Biolector system for growth at the screening concentration of chemical (38 g/L putrescine or HMDA) in biological triplicates. The average growth rate and lag time for the three replicates are shown in Tables 7 (putrescine) and 8 (HMDA), indicating standard deviation about the mean for the measurement at each time point, representing the growth characteristics of the wild-type K-12 MG1655 and the isolates from each population.

Large differences in growth behavior amongst evolved isolates can be noted. Better growing strains are defined by both the higher growth rate and the reduced lag time (i.e., at what time the cultures begin growing). Some isolates exhibit poor performance (e.g. PUTR5-1 and HMDA8-10). The phenotype to genotype relationship infers mutations that are of highest interest and those that are not of interest. Another example of this would, for example, be when two strains are growing identically (e.g. HMDA3-4 and HMDA3-6). This indicates that any differences in mutations between these two isolates are not important for tolerance. For HMDA3-4 and HMDA3-6, this suggests that the intergenic mutation between gatY and fbaB does not contribute to the tolerance phenotype.

TABLE 7
Averaged growth data of biological triplicates of putrescine-evolved
isolates grown in M9 + 1% glucose + 38 g/L putrescine

| | mean (1) | | std. error (1) | | mean (2) | | std. error (2) | |
|---|---|---|---|---|---|---|---|---|
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.100 | 12.0 | 0.003 | 5.6 | 0.112 | 10.4 | 0.044 | 9.3 |
| PUTR2-4 | 0.224 | 25.0 | 0.011 | 2.6 | 0.212 | 25.6 | 0.021 | 2.6 |
| PUTR2-6 | 0.232 | 21.6 | 0.014 | 2.4 | 0.237 | 23.3 | 0.016 | 1.6 |
| PUTR3-1 | 0.326 | 14.4 | 0.008 | 1.7 | 0.370 | 16.1 | 0.010 | 1.7 |
| PUTR3-9 | 0.181 | 14.6 | 0.049 | 10.6 | 0.197 | 19.3 | 0.048 | 3.9 |
| PUTR3-10 | 0.245 | 22.8 | 0.107 | 11.2 | 0.308 | 24.9 | 0.080 | 11.3 |
| PUTR4-3 | 0.283 | 8.0 | 0.061 | 1.7 | 0.272 | 10.5 | 0.032 | 2.6 |
| PUTR4-7 | 0.287 | 14.5 | 0.019 | 1.5 | 0.259 | 17.1 | 0.014 | 2.5 |
| PUTR4-8 | 0.237 | 17.4 | 0.034 | 2.3 | 0.244 | 25.7 | 0.018 | 9.0 |
| PUTR5-1 | 0.130 | 27.5 | 0.035 | 3.9 | 0.167 | 28.1 | 0.040 | 3.0 |
| PUTR5-6 | 0.202 | 10.5 | 0.010 | 1.8 | 0.223 | 11.2 | 0.003 | 1.4 |
| PUTR5-8 | 0.199 | 8.7 | 0.002 | 2.9 | 0.214 | 10.9 | 0.008 | 0.7 |
| PUTR6-2 | 0.192 | 19.0 | 0.021 | 5.5 | 0.234 | 23.0 | 0.022 | 7.8 |
| PUTR6-7 | 0.076 | 21.7 | 0.003 | 23.8 | 0.356 | 42.4 | 0.162 | 5.7 |
| PUTR6-10 | 0.242 | 10.6 | 0.019 | 0.6 | 0.288 | 19.2 | 0.012 | 1.2 |
| PUTR7-1 | 0.267 | 9.4 | 0.019 | 0.5 | 0.242 | 12.5 | 0.029 | 0.6 |
| PUTR7-7 | 0.267 | 3.5 | 0.042 | 0.4 | 0.282 | 12.3 | 0.021 | 0.9 |
| PUTR7-9 | 0.277 | 5.3 | 0.016 | 0.8 | 0.265 | 11.4 | 0.014 | 0.8 |
| PUTR8-3 | 0.236 | 24.1 | 0.041 | 2.1 | 0.274 | 24.2 | 0.028 | 4.3 |
| PUTR8-6 | 0.284 | 9.7 | 0.020 | 2.1 | 0.342 | 11.6 | 0.039 | 0.2 |
| PUTR8-10 | 0.285 | 6.6 | 0.020 | 1.9 | 0.335 | 11.1 | 0.026 | 0.2 |

TABLE 8
Averaged growth data of biological triplicates of HMDA-evolved
isolates grown in M9 + 1% glucose + 38 g/L HMDA

| | mean (1) | | std. error (1) | | mean (2) | | std. error (2) | |
|---|---|---|---|---|---|---|---|---|
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.048 | 38.2 | 0.068 | 13.9 | 0.051 | 29.5 | 0.004 | 1.6 |
| HMDA1-10 | 0.314 | 11.6 | 0.022 | 0.8 | 0.343 | 12.3 | 0.013 | 0.6 |
| HMDA2-1 | 0.266 | 32.8 | 0.014 | 2.0 | 0.324 | 33.3 | 0.011 | 3.1 |
| HMDA2-8 | 0.251 | 29.5 | 0.034 | 5.4 | 0.338 | 30.9 | 0.022 | 3.8 |
| HMDA3-4 | 0.428 | 18.7 | 0.096 | 1.8 | 0.335 | 18.4 | 0.052 | 2.0 |
| HMDA3-5 | 0.508 | 13.0 | 0.035 | 1.0 | 0.649 | 14.4 | 0.041 | 0.9 |
| HMDA3-6 | 0.443 | 19.7 | 0.047 | 1.7 | 0.381 | 19.7 | 0.074 | 2.1 |
| HMDA4-2 | 0.380 | 15.2 | 0.034 | 9.7 | 0.407 | 18.5 | 0.062 | 9.0 |
| HMDA4-6 | 0.322 | 22.6 | 0.008 | 9.6 | 0.355 | 24.8 | 0.015 | 5.3 |
| HMDA4-9 | 0.298 | 9.7 | 0.003 | 1.7 | 0.317 | 11.8 | 0.018 | 2.8 |
| HMDA5-4 | 0.488 | 27.6 | 0.042 | 4.3 | 0.414 | 27.4 | 0.066 | 3.4 |
| HMDA5-5 | 0.393 | 32.6 | 0.003 | 1.7 | 0.390 | 34.4 | 0.078 | 1.4 |
| HMDA5-10 | 0.250 | 26.3 | 0.001 | 2.8 | 0.278 | 32.8 | 0.049 | 8.6 |
| HMDA6-3 | 0.341 | 14.3 | 0.006 | 2.2 | 0.398 | 15.3 | 0.028 | 1.1 |
| HMDA6-7 | 0.320 | 12.4 | 0.036 | 2.1 | 0.382 | 14.9 | 0.025 | 0.9 |
| HMDA7-1 | 0.490 | 5.9 | 0.010 | 0.2 | 0.387 | 6.9 | 0.012 | 0.9 |
| HMDA7-7 | 0.353 | 11.8 | 0.068 | 4.1 | 0.342 | 13.0 | 0.043 | 2.8 |
| HMDA7-10 | 0.424 | 7.2 | 0.041 | 1.0 | 0.348 | 7.4 | 0.018 | 1.1 |
| HMDA8-5 | 0.212 | 24.3 | 0.145 | 11.7 | 0.245 | 23.2 | 0.126 | 8.2 |
| HMDA8-9 | 0.176 | 8.5 | 0.249 | 3.9 | 0.197 | 16.8 | 0.140 | 13.8 |
| HMDA8-10 | 0.206 | 32.6 | 0.049 | 3.9 | 0.229 | 30.8 | 0.068 | 5.4 |

d) Sole Carbon Source Plate Growth Assay

Wild-type, putrescine, and HMDA evolved strains were struck on M9 agar containing putrescine or HMDA as a sole carbon source. No growth was observed on HMDA plates indicating that E. coli cannot utilize HMDA as a sole carbon source. E. coli is known to be able to degrade putrescine as a sole carbon source, and slow growth was observed on putrescine containing plates. After a few weeks, widely varying growth trends could be observed between strains (Table 9), which can be correlated with mutation profiles. K-12 MG1655 exhibited the most robust growth on plates, together with PUTR4-3, PUTR5-6, PUTR5-8, and PUTR6-2. Strains that possess losses-of-function in ProV or ProX are indicated in Table 9, thus it is notable that 4 out of 5 of the best growing strains still possess functional ProVWX. This is suggestive of ProVWX, an ABC transporter having known promiscuous quaternary amine import properties, being involved in putrescine import.

PUTR2-4, PUTR2-6, PUTR5-1, PUTR7-1, PUTR7-7, and PUTR7-9 possess intact ProVWX however they still exhibit impaired growth. All of these strains also possess coding mutations in mreB or murA, indicating that these genes, possibly related to changes in cell shape (see the in a later section “Flow cytometric analysis of cell morphology”), are also resulting in diminished import or catabolism of putrescine. A marked difference in ability to grow on putrescine as a sole carbon source can also be observed between PUTR8-3, which exhibits moderate growth, and PUTR8-6 and PUTR8-10 which exhibit nearly completely abolished growth. These strains have similar sets of mutations, with PUTR8-3 lacking only the frameshift mutation in nagC. NagC is a transcriptional regulator that binds N-acetylglucosamine 6-phosphate, a precursor for peptidoglycan biosynthesis, and controls the expression of genes to coordinate the biosynthesis and degradation of this component. Thus cells lacking functional NagC may also possess cell wall modifications that reduce the import or catabolism of putrescine.

A mutational correlation analysis was additionally performed by assigning a qualitative growth defect score between 1 and 10 to each strain and determining the correlation coefficient for each mutation assuming a linear model for their impact on the growth phenotype. When this is performed by minimizing the sum of the square of the residuals between the calculated and assigned values for the growth defect score, the mutated gene with the highest correlation coefficient is mreB (6.52), followed by rpoB (3.09), rpoD (2.26), and nagC (1.89), suggesting that these genes were causative for the associated growth phenotypes on putrescine as a sole carbon source. Mutations in proV, proX, or proW were found to be non-causative for growth on putrescine as a sole carbon source in this fitted model (correlation coefficient of zero), which does not account for possible genetic interactions. Thus mutations in proV, proX, or proW are likely not involved in the direct import of putrescine and mutations in MreB are likely involved in reducing intracellular levels of putrescine.

TABLE 9
Growth of sequenced putrescine-evolved isolates on M9 agar
plates containing putrescine as a sole carbon source

| | | proVWX | nagC/nagA | mreB/murA |
|---|---|---|---|---|
| | growth score | mutation | mutation | mutation |
| MG1655 | ++++ | no | no | no |
| PUTR2-4 | + | no | no | yes |
| PUTR2-6 | + | no | no | yes |
| PUTR3-1 | + | yes | no | no |
| PUTR3-9 | + | no | no | yes |
| PUTR3-10 | + | yes | no | no |
| PUTR4-3 | +++ | yes | no | no |
| PUTR4-7 | + | yes | no | no |
| PUTR4-8 | + | yes | no | no |
| PUTR5-1 | + | no | no | yes |
| PUTR5-6 | +++ | no | no | no |
| PUTR5-8 | +++ | no | no | no |
| PUTR6-2 | ++++ | no | no | no |
| PUTR6-7 | ++ | yes | no | no |
| PUTR6-10 | ++ | yes | no | no |
| PUTR7-1 | + | no | no | yes |
| PUTR7-7 | + | no | no | yes |
| PUTR7-9 | + | no | no | yes |
| PUTR8-3 | ++ | yes | no | yes |
| PUTR8-6 | none | yes | yes | yes |
| PUTR8-10 | none | yes | yes | yes |

e) Knockout Strain Growth Performance—High Putrescine Concentrations

Group 1 (probable loss-of-function) mutations were identified from re-sequencing results as described in methods. Two different frameshift mutations were present in proV and one frameshift mutation was present in proX in populations 3, 4, 6, and 8, respectively (proV and proX encode different subunits of the same protein). Frameshift mutations and insertion sequence elements were identified in cspC in populations 2 and 4, and an insertion sequence element was identified in population 6 in yobF, a protein of unknown function found in the same operon as cspC. Two different frameshift deletions were identified in nagC in population 8. Insertion sequence elements were identified in yeaR in population 6. Two different frameshift mutations were identified in individual isolates in populations 3 and 5. Any additional mutations tested for imparting putrescine tolerance were identified in HMDA-evolved strains (description follows) and were also tested in putrescine due to the similarity of the two chemicals and similar sets of genes being mutated following evolution. Combinations of mutations were selected partly based on the presence of particular mutations with each other, so some gene disruptions were not tested alone (e.g. nagC).

Initially, single knockouts and a few double knockout combinations were screened with the Growth Profiler at two concentrations: 19 g/L and 38 g/L putrescine. Growth data for individual biological replicates are shown in Table 10.

In this testing format, it was found that of the knockouts tested, deletion of proV significantly increased the growth rate at 19 g/L and decreased the lag time in 38 g/L. Double knockouts, which all contained a deletion of proV and another gene, did not appear to exhibit improved growth relative to the proV deletion alone.

Strains including additional double and triple knockout strains that had been constructed based on both these Growth Profiler results and mutations found in HMDA evolved strains (see data below) were then tested in the Biolector testing format together with a selection of evolved strains (Table 11).

The best performing strains were the proV single deletion strain and the proV cspC double deletion strain. Using the original algorithm (see section 17), the proV yobF double-deletion strain was possible also among them (although significant variation between replicates). It should be noted that cspC and yobF are transcribed in the same operon, so there is a possibility that disruption of one affects expression of the other, and/or that they are related in function and are possibly even involved in the same overall cellular response.

A second Biolector experiment was performed repeating growth of several of the strains shown in the first experiment but also including a few additional strains (Table 12).

The best performing strain in terms of lag time in this run was the cspC single knockout, while the strain with the highest growth rate but non-improved lag time was the ptsP single knockout. Reduced lag times were also apparent in the proV single knockout, proV cspC double knockout, proV ptsP double knockout, and proV ptsP wbbK triple knockout strains.

The Keio collection of gene knockouts is a commercial collection of knockouts in nearly all non-essential genes and ORFs in E. coli strain BW25113. This strain is a K-12 derivative and possesses known mutations relative to the K-12 MG1655 background. All Keio collection strains with knockouts in genes that were found to be mutated in Table 5, minus knockouts in genes that were already screened in the K-12 MG1655 background in Tables 10, 11, and 12, were screened for growth against the BW25113 control in M9+1% glucose+32 g/L and also plus 38 g/L putrescine in the Growth Profiler screening format. Averaged growth data for 3 biological replicate cultures were calculated for each strain at 32 g/L and 38 g/L (Table 13). No significant improvements in growth rate or reductions in lag time were observed.

A list of all gene disruption mutants in both the K-12 MG1655 and BW25113 background strains that exhibited increased tolerance to putrescine is shown in Table 14.

TABLE 10
Preliminary screening of predicted loss-of-function mutations found
in evolved strains in different concentrations of putrescine

| | 19 g/L putrescine | | | | 38 g/L putrescine | | | |
|---|---|---|---|---|---|---|---|---|
| | mean (1) | | std. error (1) | | mean (1) | | std. error (1) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.528 | 2.3 | 0.045 | 0.6 | 0.244 | 6.6 | 0.008 | 1.4 |
| MG1655 proV::kan | 0.586 | 2.1 | 0.027 | 0.3 | 0.197 | 5.9 | 0.011 | 5.0 |
| MG1655 cspC::kan | 0.531 | 1.9 | 0.056 | 0.7 | 0.125 | 16.6 | 0.007 | 2.2 |
| MG1655 yeaR::kan | 0.535 | 2.2 | 0.020 | 0.4 | 0.212 | 4.5 | 0.015 | 0.8 |
| MG1655 pstS::kan | 0.456 | 3.2 | 0.024 | 1.7 | 0.176 | 10.2 | 0.033 | 0.1 |
| MG1655 ΔproV cspC::kan | 0.613 | 2.0 | 0.040 | 0.6 | 0.151 | 21.0 | 0.005 | 6.3 |
| MG1655 ΔproV yobF::kan | 0.562 | 1.9 | 0.010 | 0.3 | 0.211 | 9.8 | 0.011 | 4.2 |
| MG1655 ΔproV nagC::kan | 0.522 | 1.8 | 0.008 | 0.6 | 0.186 | 6.6 | 0.022 | 5.3 |
| | mean (2) | | std. error (2) | | mean (2) | | std. error (2) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.457 | 7.9 | 0.015 | 0.2 | 0.172 | 22.6 | 0.009 | 0.4 |
| MG1655 proV::kan | 0.535 | 7.0 | 0.009 | 0.2 | 0.161 | 21.1 | 0.012 | 0.3 |
| MG1655 cspC::kan | 0.453 | 7.7 | 0.011 | 0.1 | 0.157 | 38.6 | 0.077 | 6.8 |
| MG1655 yeaR::kan | 0.459 | 8.1 | 0.011 | 0.0 | 0.156 | 23.6 | 0.003 | 0.5 |
| MG1655 pstS::kan | 0.322 | 8.9 | 0.010 | 0.5 | 0.085 | 26.3 | 0.024 | 1.9 |
| MG1655 ΔproV cspC::kan | 0.533 | 7.1 | 0.008 | 0.1 | 0.142 | 34.5 | 0.005 | 1.3 |
| MG1655 ΔproV yobF::kan | 0.534 | 7.2 | 0.005 | 0.0 | 0.175 | 25.5 | 0.009 | 0.1 |
| MG1655 ΔproV nagC::kan | 0.456 | 7.8 | 0.006 | 0.3 | 0.123 | 25.8 | 0.028 | 3.0 |

TABLE 11
First Biolector growth screen of loss-of-function mutants inferred from putrescine evolved isolates

| | mean (1) | | std. error (1) | | mean (2) | | std. error (2) | |
|---|---|---|---|---|---|---|---|---|
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.110 | 17.6 | 0.002 | 1.2 | 0.105 | 18.9 | 0.020 | 3.5 |
| PUTR3-1 | 0.374 | 11.8 | 0.013 | 0.5 | 0.372 | 13.2 | 0.014 | 0.3 |
| PUTR4-3 | 0.335 | 6.7 | 0.039 | 0.8 | 0.267 | 9.0 | 0.005 | 0.2 |
| PUTR5-6 | 0.221 | 5.6 | 0.012 | 0.0 | 0.240 | 9.2 | 0.000 | 0.4 |
| PUTR7-7 | 0.316 | — | 0.024 | — | 0.332 | 9.1 | 0.011 | 1.4 |
| PUTR8-10 | 0.318 | 5.6 | 0.003 | 0.7 | 0.340 | 19.0 | 0.045 | 18.0 |
| MG1655 cspC::kan | 0.133 | 20.5 | 0.010 | 1.4 | 0.103 | 14.6 | 0.013 | 1.5 |
| MG1655 proV::kan | 0.105 | 24.4 | 0.008 | 2.8 | 0.127 | 28.7 | 0.017 | 3.2 |
| MG1655 yeaR::kan | 0.071 | 24.2 | — | — | 0.078 | 31.0 | 0.017 | 26.1 |
| MG1655 pstS::kan | 0.102 | 14.9 | 0.009 | 3.1 | 0.068 | 17.4 | 0.009 | 1.2 |
| MG1655 ΔproV cspC::kan | 0.147 | 21.6 | 0.021 | 1.6 | 0.121 | 27.7 | 0.060 | 5.9 |
| MG1655 ΔproV yobF::kan | 0.132 | 20.5 | 0.039 | 0.1 | 0.105 | 27.2 | 0.054 | 11.6 |
| MG1655 ΔproV nagC::kan | 0.135 | 17.3 | 0.018 | 2.4 | 0.094 | 26.5 | 0.053 | 2.6 |
| MG1655 ΔproV wbbK::kan | 0.112 | 17.0 | 0.031 | 1.1 | 0.115 | 18.0 | 0.023 | 7.2 |
| MG1655 ΔproV ptsP::kan | 0.100 | 27.8 | 0.021 | 1.7 | 0.088 | 32.8 | 0.026 | 2.8 |

TABLE 12
Second Biolector growth screen of loss-of-function mutants inferred from putrescine evolved isolates
A:

| | | | | | mean | | std. error | |
|---|---|---|---|---|---|---|---|---|
| | mean (1) | | std. error (1) | | (phase 2) (1) | | (phase 2) (1) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.118 | 39.9 | 0.016 | 0.6 | 0.121 | 14.5 | 0.016 | 1.5 |
| PUTR3-1 | 0.400 | 10.0 | 0.019 | 0.6 | — | — | — | — |
| PUTR4-3 | 0.351 | 4.6 | 0.040 | 1.1 | — | — | — | — |
| PUTR5-6 | 0.205 | 1.9 | 0.011 | 0.5 | — | — | — | — |
| PUTR7-7 | 0.312 | 2.0 | 0.055 | 1.8 | — | — | — | — |
| PUTR8-10 | 0.304 | 4.8 | 0.012 | 1.6 | — | — | — | — |
| MG1655 proV::kan | 0.136 | 20.7 | 0.008 | 1.8 | — | — | — | — |
| MG1655 cspC::kan | 0.160 | 17.3 | 0.008 | 1.1 | — | — | — | — |
| MG1655 ptsP::kan | 0.177 | 29.6 | 0.023 | 3.9 | — | — | — | — |
| MG1655 wbbK::kan | 0.099 | 38.9 | 0.011 | 3.1 | 0.101 | 12.6 | 0.029 | 4.7 |
| MG1655 ΔproV cspC::kan | 0.149 | 24.8 | 0.004 | 5.2 | — | — | — | — |
| MG1655 ΔproV nagC::kan | 0.119 | 38.6 | 0.019 | 3.0 | 0.143 | 10.2 | 0.000 | 0.2 |
| MG1655 ΔproV ptsP::kan | 0.139 | 20.1 | 0.042 | 9.2 | — | — | — | — |
| MG1655 ΔproV wbbK::kan | 0.111 | 29.3 | 0.034 | 12.0 | 0.128 | 14.2 | 0.002 | 0.8 |
| MG1655 ΔproV ΔptsP wbbK::kan | 0.151 | 22.3 | 0.008 | 1.8 | — | — | — | — |
| MG1655 ΔproV ΔptsP nagC::kan | 0.152 | 35.1 | 0.008 | 7.7 | 0.114 | 16.6 | 0.036 | 3.4 |

B:

| | |
|---|---|
| mean (2) | std. error (2) |

| strain | μ (h⁻¹) | t_{lag} (h) | μ (h⁻¹) | t_{lag} (h) |
|---|---|---|---|---|
| MG1655 | 0.171 | 32.5 | 0.024 | 1.9 |
| PUTR3-1 | 0.390 | 13.1 | 0.016 | 0.2 |
| PUTR4-3 | 0.261 | 7.1 | 0.002 | 0.2 |
| PUTR5-6 | 0.215 | 6.7 | 0.004 | 0.3 |
| PUTR7-7 | 0.297 | 8.7 | 0.021 | 0.3 |
| PUTR8-10 | 0.354 | 8.6 | 0.007 | 0.2 |
| MG1655 proV::kan | 0.145 | 24.9 | 0.009 | 1.5 |
| MG1655 cspC::kan | 0.125 | 14.8 | 0.003 | 0.6 |
| MG1655 ptsP::kan | 0.208 | 31.4 | 0.082 | 5.0 |
| MG1655 wbbK::kan | 0.131 | 32.1 | 0.013 | 4.1 |
| MG1655 ΔproV cspC::kan | 0.143 | 24.4 | 0.021 | 7.0 |
| MG1655 ΔproV nagC::kan | 0.170 | 35.7 | 0.018 | 1.5 |
| MG1655 ΔproV ptsP::kan | 0.152 | 25.5 | 0.014 | 3.8 |
| MG1655 ΔproV wbbK::kan | 0.132 | 19.7 | 0.041 | 10.8 |
| MG1655 ΔproV ΔptsP wbbK::kan | 0.140 | 22.4 | 0.006 | 1.9 |
| MG1655 ΔproV ΔptsP nagC::kan | 0.174 | 34.3 | 0.030 | 2.4 |

TABLE 13
Keio collection mutants exhibiting qualitatively improved growth
in Growth Profiler screening with 32 g/L and 38 g/L putrescine

| | 32 g/L putrescine | | | | 38 g/L putrescine | | | |
|---|---|---|---|---|---|---|---|---|
| | mean (1) | | std. error (1) | | mean (1) | | std. error (1) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| BW25113 | 0.336 | 3.9 | 0.002 | 1.4 | 0.248 | 8.0 | 0.002 | 1.3 |
| BW25113 yjhG::kan | 0.245 | 5.8 | 0.037 | 0.5 | — | — | — | — |
| BW25113 ygcE::kan | 0.284 | 3.8 | 0.021 | 2.7 | 0.167 | 12.9 | 0.026 | 9.6 |
| BW25113 waaS::kan | — | — | — | — | 0.280 | 7.7 | 0.018 | 1.5 |
| BW25113 rpoD::kan | 0.296 | 3.4 | 0.002 | 1.1 | 0.238 | 5.1 | 0.018 | 2.4 |
| BW25113 sfmH::kan | 0.310 | 3.3 | 0.010 | 2.7 | 0.179 | 4.2 | 0.027 | 5.0 |
| BW25113 tdcR::kan | 0.352 | 2.4 | 0.027 | 1.1 | 0.214 | 7.0 | 0.014 | 0.6 |
| BW25113 proX::kan | 0.303 | 2.8 | — | — | 0.285 | 8.3 | 0.039 | 1.2 |
| BW25113 yobF::kan | 0.339 | 3.2 | 0.030 | 0.9 | 0.226 | 8.1 | 0.075 | 2.6 |
| BW25113 ytfR::kan | 0.289 | 4.1 | 0.006 | 1.2 | 0.192 | 9.8 | 0.032 | 2.4 |
| BW25113 rph::kan | 0.311 | 2.3 | 0.061 | 1.0 | 0.205 | 6.5 | 0.023 | 4.2 |
| BW25113 mdtJ::kan | 0.288 | 3.6 | 0.002 | 0.6 | 0.276 | 5.9 | 0.007 | 1.9 |
| BW25113 yicC::kan | 0.375 | 5.1 | 0.037 | 5.0 | 0.286 | 8.7 | 0.007 | 0.9 |
| BW25113 essD::kan | 0.241 | 5.0 | 0.067 | 1.3 | 0.257 | 11.6 | — | — |
| BW25113 yjcF::kan | 0.301 | 3.5 | 0.032 | 0.7 | 0.225 | 7.0 | 0.032 | 1.4 |
| BW25113 iscR::kan | 0.295 | 3.6 | 0.006 | 0.4 | 0.195 | 6.2 | 0.018 | 0.3 |
| BW25113 yedP::kan | 0.337 | 5.0 | 0.039 | 0.5 | 0.234 | 5.4 | 0.038 | 1.8 |
| | mean (2) | | std. error (2) | | mean (2) | | std. error (2) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| BW25113 | 0.308 | 12.9 | 0.012 | 0.1 | 0.198 | 22.6 | 0.029 | 1.2 |
| BW25113 yjhG::kan | 0.209 | 18.7 | 0.023 | 1.1 | 0.107 | 35.6 | 0.052 | 10.3 |
| BW25113 ygcE::kan | 0.276 | 13.8 | 0.026 | 1.0 | 0.180 | 22.6 | 0.026 | 1.4 |
| BW25113 waaS::kan | 0.259 | 13.9 | 0.013 | 0.2 | 0.181 | 23.3 | 0.004 | 0.2 |
| BW25113 rpoD::kan | 0.246 | 23.9 | 0.086 | 18.7 | 0.209 | 21.1 | 0.010 | 0.1 |
| BW25113 sfmH::kan | 0.285 | 13.9 | 0.072 | 3.2 | 0.185 | 23.5 | 0.049 | 6.5 |
| BW25113 tdcR::kan | 0.270 | 13.6 | 0.010 | 0.1 | 0.196 | 21.2 | 0.009 | 0.4 |
| BW25113 proX::kan | 0.280 | 13.4 | 0.025 | 0.6 | 0.162 | 22.9 | 0.024 | 0.5 |
| BW25113 yobF::kan | 0.293 | 13.6 | 0.009 | 0.3 | 0.173 | 22.4 | 0.006 | 1.0 |
| BW25113 ytfR::kan | 0.286 | 13.6 | 0.009 | 0.2 | 0.187 | 22.1 | 0.019 | 0.5 |
| BW25113 rph::kan | 0.289 | 13.8 | 0.003 | 0.1 | 0.157 | 23.9 | 0.002 | 0.5 |
| BW25113 mdtJ::kan | 0.256 | 15.6 | 0.029 | 3.1 | 0.139 | 33.3 | 0.048 | 14.3 |
| BW25113 yicC::kan | 0.299 | 13.4 | 0.007 | 0.7 | 0.199 | 22.4 | 0.022 | 1.4 |
| BW25113 essD::kan | 0.262 | 14.4 | 0.026 | 0.6 | 0.188 | 22.9 | 0.008 | 0.4 |
| BW25113 yjcF::kan | 0.215 | 19.1 | 0.031 | 1.2 | 0.141 | 32.3 | 0.016 | 2.5 |
| BW25113 iscR::kan | 0.301 | 13.4 | 0.002 | 0.3 | 0.192 | 22.9 | 0.015 | 1.0 |
| BW25113 yedP::kan | 0.263 | 14.5 | 0.014 | 0.5 | 0.155 | 28.5 | 0.008 | 0.4 |

TABLE 14
Gene deletion mutants exhibiting improved
growth in high concentrations of putrescine

| Strain genotype | Improved growth parameter |
|---|---|
| K-12 MG1655 proV::kan | Growth rate and/or lag time |
| K-12 MG1655 cspC::kan | Lag time |
| K-12 MG1655 ptsP::kan | Growth rate |
| K-12 MG1655 ΔproV wbbK::kan | Lag time |
| K-12 MG1655 ΔproV cspC::kan | Growth rate and/or lag time |
| K-12 MG1655 ΔproV yobF: :kan | Growth rate |
| K-12 MG1655 ΔproV ptsP::kan | Lag time |
| K-12 MG1655 ΔproV ΔptsP wbbK::kan | Llag time |

The strains in Table 15 are a list of those tested but that were assessed to offer no significant improvement in growth in high concentrations of putrescine.

TABLE 15
Gene deletion mutants that were tested but did not
improve growth in high concentrations of putrescine

| | Growth rate | Lag time |
|---|---|---|
| Strain genotype | improvement | improvement |
| K-12 MG1655 yeaR::kan | none | none |
| K-12 MG1655 pstS::kan | none | none |
| K-12 MG1655 wbbK::kan | none | none |
| K-12 MG1655 ΔproV nagC::kan | negative | negative |
| K-12 MG1655 ΔproV ΔptsP nagC::kan | negative | negative |
| All available Keio collection strains with | none | none |
| knockouts in individual genes shown in | | |
| Table 5, except for those listed in Table | | |
| 14 | | |

f) Knockout Strain Performance—HMDA

Two different frameshift mutations and two different insertion sequence elements in proV were identified in populations 1, 2, 4, 6, and 8. Another large deletion spanning proV and part of proW was also identified in population 3. Insertion sequence elements and SNPs generating premature stop codons in nagC were present in isolates from populations 3 and 5, and all isolates from population 8. A frameshift mutation and coding SNP were identified in nagA in populations 3 (the isolates that did not have the nagC mutation) and 6. One frameshift mutation and one coding SNP were identified in ptsP in populations 2 and 5. A frameshift mutation in wbbK was found in population 7. Any additional mutations tested for imparting HMDA tolerance were identified in putrescine-evolved strains (description follows) and were also tested in HMDA due to the similarity of the two chemicals and similar sets of genes being mutated following evolution. A nagA deletion mutant was not tested due to previous work in our lab showing that this deletion mutant behaves very similarly to the nagC deletion mutant, with both genes involved in the same pathway. We previously isolated transposon insertion mutants of nagC and nagA from a library in E. coli W following selection on 0.6 M NaCl and confirmed improved growth of clean deletion mutants in that condition (Lennen and Herrgård, Appl. Environ. Microbiol., 2014).

Initially, single knockouts and a few double knockout combinations were screened in the Growth Profiler at two concentrations: 19 g/L and 38 g/L HMDA. Growth data are shown in Table 16.

In this testing format, all strains shown in Table 16 exhibited improved growth at 19 g/L, with increased growth rates and equivalent or reduced lag times. At 38 g/L, only the proV and ptsP single knockout strains, and proV+ptsP double knockout strains exhibited improved growth, with both increased growth rates and decreased lag times. The proV nagC double knockout strain notably exhibited completely abolished growth in 38 g/L HMDA. The proV+ptsP double knockout exhibited a much higher growth rate than other strains.

Based on the results from this first run in the Growth Profiler, some additional combination knockout strains were constructed and tested in the same format. The growth data for 38 g/L based on the averaged of three biological replicates with the standard deviation between values at each timepoint are shown in Table 17.

It was observed that of the strains tested, K-12 ΔproV ptsP::kan remained the best growing strain. The proV wbbK double knockout strain exhibited a slight improvement in growth rate and reduction in lag time compared to the proV single knockout.

Strains including an additional triple knockout combination based on the Growth Profiler results were then tested in the Biolector testing format in two separate experiments together with a selection of evolved strains (Table 18).

Greatly improved growth over the wild-type was observed for the proV and ptsP single deletion mutants, with moderately improved growth for the wbbK single deletion mutant. The proV wbbK double mutant performed worse than the proV single mutant, however the proV ptsP wbbK triple mutant performed both better than both combinations of double mutants. Due to some large variations between replicates for many strains, the strain genotypes were confirmed by colony PCR and the experiment was also repeated again on another date (Table 19).

Again, the proV and ptsP single mutants exhibited significantly improved growth compared to the wild-type, with the wbbK mutant exhibiting a small improvement. The proV ptsP and proV ptsP wbbK double and triple mutants performed better than the proV wbbK double deletion strain, which also performed significantly worse than the proV single deletion strain. An improved growth rate was observed in the proV ptsP nagC triple deletion mutant under this growth condition. A list of the gene disruption mutations that were found to provide increased tolerance to HMDA is shown in Table 21, and tested mutants that did not improve growth are listed in Table 22.

All Keio collection strains with knockouts in genes that were found to be mutated in Table 2 were screened for growth against the BW25113 control in M9+1% glucose+32 g/L and also plus 38 g/L HMDA in the Growth Profiler screening format. Averaged growth curves for 3 biological replicate cultures are shown individually for each strain at 32 g/L and 38 g/L (Table 20). Moderate to large improvements in growth rate and lag time was observed in the mpl, rph, and ybeX deletion strains in 38 g/L HMDA, and for the rph and ybeX deletion strains in 32 g/L HMDA.

TABLE 16
Preliminary screening of predicted loss-of-function mutations
found in evolved strains in different concentrations of HMDA

| | 19 g/L HMDA | | | | 38 g/L HMDA | | | |
|---|---|---|---|---|---|---|---|---|
| | mean (1) | | std. error (1) | | mean (1) | | std. error (1) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.550 | 2.1 | 0.035 | 0.5 | 0.119 | 33.1 | 0.011 | 2.1 |
| MG1655 proV::kan | 0.633 | 2.2 | 0.022 | 0.3 | 0.184 | 28.6 | 0.064 | 6.0 |
| MG1655 ptsP::kan | 0.622 | 2.2 | 0.023 | 0.1 | 0.138 | 22.8 | 0.011 | 0.9 |
| MG1655 wbbK::kan | 0.509 | 2.2 | 0.026 | 0.2 | 0.133 | 34.5 | 0.034 | 4.4 |
| MG1655 ΔproV nagC::kan | 0.543 | 1.8 | 0.038 | 0.3 | 0.000 | — | 0.000 | — |
| MG1655 ΔproV ptsP::kan | 0.729 | 2.6 | 0.035 | 0.3 | 0.299 | 33.0 | 0.016 | 3.0 |
| | mean (2) | | std. error (2) | | mean (2) | | std. error (2) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.451 | 7.5 | 0.017 | 0.2 | 0.134 | 47.4 | 0.024 | 0.9 |
| MG1655 proV::kan | 0.545 | 6.8 | 0.051 | 0.1 | 0.194 | 36.5 | 0.041 | 2.6 |
| MG1655 ptsP::kan | 0.520 | 6.8 | 0.020 | 0.1 | 0.154 | 37.4 | 0.039 | 2.4 |
| MG1655 wbbK::kan | 0.478 | 7.3 | 0.016 | 0.1 | 0.101 | 42.5 | 0.011 | 0.7 |
| MG1655 ΔproV nagC::kan | 0.525 | 7.6 | 0.018 | 0.1 | 0.000 | — | 0.000 | — |
| MG1655 ΔproV ptsP::kan | 0.658 | 6.4 | 0.009 | 0.1 | 0.331 | 37.2 | 0.007 | 2.1 |

TABLE 17
Second preliminary screening of predicted loss-of-function mutations
found in evolved strains in different concentrations of HMDA

| | 19 g/L HMDA | | | | 38 g/L HMDA | | | |
|---|---|---|---|---|---|---|---|---|
| | mean (1) | | std. error (1) | | mean (1) | | std. error (1) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.569 | 4.0 | 0.011 | 0.2 | 0.197 | 2.4 | 0.053 | 4.8 |
| MG1655 proV::kan | 0.586 | 4.1 | 0.028 | 0.3 | 0.307 | 3.3 | 0.026 | 1.9 |
| MG1655 ptsP::kan | 0.558 | 4.3 | 0.029 | 0.9 | 0.284 | 5.0 | 0.022 | 1.1 |
| MG1655 wbbK::kan | 0.575 | 3.8 | 0.081 | 0.6 | 0.209 | 7.3 | 0.026 | 8.3 |
| MG1655 ΔproV ptsP::kan | 0.667 | 3.5 | 0.030 | 0.2 | 0.308 | 3.7 | 0.022 | 0.7 |
| MG1655 ΔproV wbbK::kan | 0.650 | 3.5 | 0.040 | 0.2 | 0.317 | 5.4 | 0.018 | 0.8 |
| MG1655 ΔproV nagC::kan | 0.599 | 3.7 | 0.025 | 0.4 | 0.279 | 3.4 | 0.014 | 0.2 |
| MG1655 ΔproV ΔptsP wbbK::kan | 0.732 | 3.7 | 0.027 | 0.2 | 0.320 | 11.9 | 0.019 | 3.7 |
| MG1655 ΔproV ΔptsP nagC::kan | 0.630 | 3.8 | 0.029 | 0.1 | 0.247 | 5.8 | 0.016 | 1.8 |
| | mean (2) | | std. error (2) | | mean (2) | | std. error (2) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.496 | 7.7 | 0.004 | 0.0 | 0.130 | 19.8 | 0.002 | 0.9 |
| MG1655 proV::kan | 0.553 | 8.8 | 0.025 | 0.1 | 0.238 | 17.8 | 0.013 | 0.1 |
| MG1655 ptsP::kan | 0.554 | 8.7 | 0.033 | 0.9 | 0.232 | 17.5 | 0.004 | 1.7 |
| MG1655 wbbK::kan | 0.479 | 8.5 | 0.022 | 1.9 | 0.141 | 18.8 | 0.005 | 4.4 |
| MG1655 ΔproV ptsP::kan | 0.619 | 6.6 | 0.024 | 0.0 | 0.343 | 13.6 | 0.004 | 0.1 |
| MG1655 ΔproV wbbK::kan | 0.612 | 7.0 | 0.008 | 0.1 | 0.256 | 15.4 | 0.007 | 1.1 |
| MG1655 ΔproV nagC::kan | 0.525 | 7.1 | 0.011 | 0.0 | 0.216 | 15.7 | 0.004 | 0.5 |
| MG1655 ΔproV ΔptsP wbbK::kan | 0.571 | 6.6 | 0.047 | 0.2 | 0.298 | 17.7 | 0.001 | 1.8 |
| MG1655 ΔproV ΔptsP nagC::kan | 0.537 | 7.1 | 0.008 | 0.1 | 0.238 | 20.3 | 0.002 | 0.9 |

TABLE 18
First Biolector growth screen of loss-of-function mutants inferred from HMDA evolved isolates

| | mean (1) | | std. error (1) | | mean (2) | | std. error (2) | |
|---|---|---|---|---|---|---|---|---|
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.081 | 39.4 | 0.004 | 4.8 | 0.099 | 37.1 | 0.009 | 1.4 |
| HMDA1-10 | 0.370 | 5.7 | 0.031 | 0.8 | 0.407 | 7.5 | 0.003 | 0.3 |
| HMDA3-5 | 0.504 | 7.7 | 0.021 | 0.7 | 0.627 | 10.0 | 0.033 | 0.7 |
| HMDA5-4 | 0.516 | 10.5 | 0.023 | 1.3 | 0.710 | 13.3 | 0.033 | 1.5 |
| HMDA7-1 | 0.522 | 5.3 | 0.007 | 0.5 | 0.397 | 5.2 | 0.012 | 0.2 |
| MG1655 proV::kan | 0.174 | 13.9 | 0.001 | 4.6 | 0.205 | 19.0 | 0.005 | 0.9 |
| MG1655 ptsP::kan | 0.174 | 13.1 | 0.005 | 3.6 | 0.208 | 17.7 | 0.003 | 1.2 |
| MG1655 wbbK::kan | 0.112 | 29.7 | 0.004 | 2.4 | 0.128 | 26.2 | 0.005 | 1.3 |
| MG1655 ΔproV nagC::kan | 0.105 | 33.7 | 0.005 | 5.9 | 0.139 | 33.2 | 0.013 | 4.4 |
| MG1655 ΔproV ptsP::kan | 0.208 | 20.6 | 0.016 | 1.6 | 0.216 | 22.1 | 0.013 | 1.4 |
| MG1655 ΔproV wbbK::kan | 0.135 | 29.5 | 0.040 | 7.0 | 0.149 | 29.2 | 0.054 | 1.3 |
| MG1655 ΔproV ΔptsP wbbK::kan | 0.213 | 12.7 | 0.015 | 5.3 | 0.250 | 17.5 | 0.023 | 1.6 |
| MG1655 ΔproV ΔptsP nagC::kan | 0.248 | 25.6 | 0.027 | 4.1 | 0.297 | 25.5 | 0.000 | 1.3 |

TABLE 19
Second Biolector growth screen of loss-of-function mutants inferred from HMDA evolved isolates

| | mean (1) | | std. error (1) | | mean (2) | | std. error (2) | |
|---|---|---|---|---|---|---|---|---|
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.098 | 33.3 | 0.029 | 5.3 | 0.078 | 22.9 | 0.028 | 10.0 |
| HMDA1-10 | 0.392 | 6.6 | 0.045 | 1.1 | 0.407 | 7.7 | 0.034 | 0.7 |
| HMDA3-5 | 0.527 | 10.4 | 0.016 | 0.4 | 0.670 | 11.5 | 0.015 | 0.3 |
| HMDA5-4 | 0.522 | 13.1 | 0.014 | 0.3 | 0.754 | 15.4 | 0.097 | 0.2 |
| HMDA7-1 | 0.441 | 6.0 | 0.038 | 0.1 | 0.419 | 6.1 | 0.020 | 0.7 |
| MG1655 proV::kan | 0.145 | 18.1 | 0.009 | 0.6 | 0.124 | 16.7 | 0.017 | 1.5 |
| MG1655 ptsP::kan | 0.159 | 18.4 | 0.004 | 1.8 | 0.153 | 18.4 | 0.011 | 1.5 |
| MG1655 wbbK::kan | 0.095 | 26.5 | 0.004 | 3.1 | 0.088 | 19.4 | 0.013 | 1.6 |
| MG1655 ΔproV nagC::kan | 0.000 | — | 0.000 | — | 0.154 | 58.0 | 0.008 | 4.0 |
| MG1655 ΔproV ptsP::kan | 0.137 | 23.5 | 0.047 | 4.5 | 0.155 | 20.1 | 0.011 | 0.1 |
| MG1655 ΔproV wbbK::kan | 0.117 | 28.2 | 0.018 | 8.1 | 0.114 | 23.4 | 0.012 | 2.0 |
| MG1655 ΔproV ΔnagC ptsP::kan | 0.173 | 25.8 | 0.062 | 6.0 | 0.227 | 23.6 | 0.025 | 1.1 |
| MG1655 ΔproV ΔptsP wbbK::kan | 0.148 | 28.4 | 0.034 | 11.5 | 0.165 | 20.6 | 0.022 | 0.1 |

TABLE 20
Keio collection mutants exhibiting qualitatively improved growth in preliminary Growth
Profiler screening (either with 32 g/L or 38 g/L HMDA) grown in 32 g/L or 38 g/L HMDA

| | 32 g/L HMDA | | | | 38 g/L HMDA | | | |
|---|---|---|---|---|---|---|---|---|
| | mean (1) | | std. error (1) | | mean (1) | | std. error (1) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| BW25113 | 0.329 | 4.6 | 0.021 | 0.8 | 0.136 | 22.0 | 0.009 | 3.1 |
| BW25113 ybeX::kan | 0.422 | 3.8 | 0.068 | 0.3 | 0.268 | 6.0 | 0.038 | 2.2 |
| BW25113 mpl::kan | 0.287 | 5.1 | 0.011 | 2.0 | 0.129 | 10.8 | 0.009 | 6.8 |
| BW25113 pstB::kan | 0.356 | 3.7 | 0.057 | 1.2 | 0.107 | 32.8 | 0.023 | 0.0 |
| BW25113 rph::kan | 0.334 | 3.4 | 0.007 | 0.2 | 0.122 | 18.5 | 0.010 | 2.6 |
| | mean (2) | | std. error (2) | | mean (2) | | std. error (2) | |
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| BW25113 | 0.271 | 14.7 | 0.005 | 0.2 | 0.105 | 46.3 | 0.025 | 1.4 |
| BW25113 mpl::kan | 0.232 | 16.6 | 0.029 | 2.0 | 0.119 | 38.2 | 0.014 | 2.4 |
| BW25113 pstB::kan | 0.260 | 14.1 | 0.014 | 0.7 | 0.110 | 43.7 | 0.038 | 4.0 |
| BW25113 rph::kan | 0.329 | 12.7 | 0.006 | 0.2 | 0.131 | 38.0 | 0.023 | 0.0 |
| BW25113 ybeX::kan | 0.387 | 12.0 | 0.011 | 0.1 | 0.179 | 19.9 | 0.002 | 0.3 |

TABLE 21
Gene deletion mutants exhibiting improved
growth in high concentrations of HMDA

| Strain genotype | Improved growth parameter |
|---|---|
| K-12 MG1655 proV::kan | Growth rate and lag time |
| K-12 MG1655 ptsP::kan | Growth rate and lag time |
| K-12 MG1655 wbbK::kan | Growth rate and lag time |
| K-12 MG1655 ΔproV ptsP::kan | Growth rate and lag time |
| K-12 MG1655 ΔproV ΔptsP wbbK::kan | Growth rate and lag time |
| K-12 MG1655 ΔproV ΔptsP nagC::kan | Growth rate and lag time |
| BW25113 ybeX::kan | Growth rate and lag time |
| BW25113 mpl::kan | Growth rate and lag time |
| BW25113 rph::kan | Growth rate and lae time |

TABLE 22
Tested gene deletion mutants that did not exhibit
improved growth (or improved growth over single
or double mutants when in higher combinations)

| | Growth rate | Lag time |
|---|---|---|
| Strain genotype | improvement | improvement |
| K-12 MG1655 ΔproV nagC::kan | negative to | negative to |
| | neutral | neutral |
| K-12 MG1655 ΔproV wbbK::kan | negative vs. | negative vs. |
| | ΔproV | ΔproV |
| All available Keio collection strains | None | none |
| with individual knockouts in genes | | |
| shown in Table 6, except for those | | |
| listed in Table 21 | | |

A summary of the genes discussed thus far with a description of the known gene function is included in Table 23.

TABLE 23
Descriptions of genes disrupted in mutants with improved
growth in high concentrations of putrescine and HMDA

| Gene | | |
|---|---|---|
| name | Description | Notes |
| proV | Glycine betaine/proline ABC transporter periplasmic | Other subunits of the same protein are |
| | binding protein | ProW and ProX |
| cspC | Multicopy suppressor of mukB; cold shock protein | In the same operon as yobF |
| | homolog constitutively expressed at 37 C.; | |
| | antitermination protein; affects rpoS and uspA | |
| | expression | |
| ptsP | PTS PEP-protein phosphotransferase Enzyme I (Ntr) | |
| wbbK | Involved in lipopolysaccharide biosynthesis | |
| yobF | DUF2527 family heat-induced protein, function | In the same operon as cspC |
| | unknown | |
| nagC | N-acetylglucosamine-inducible nag divergent operon | In the same operon as nagA |
| | transcriptional repressor | |
| rph | Pseudogene reconstruction, RNase PH | likely due to increased transcription of |
| | | downstream pyrE* |
| yicC | UPF0701 family protein, function unknown | In divergent operon from rph, may also |
| | | be related to pyrE expression* |
| yjcF | Pentapeptide repeats protein, function unknown | |
| iscR | isc operon transcriptional repressor; suf operon | |
| | transcriptional activator; icsR regulon regulator; | |
| | oxidative stress- and iron starvation-inducible; | |
| | autorepressor; contains Fe—S cluster | |
| yedP | Predicted mannosyl-3-phosphoglycerate phosphatase; | |
| | function unknown; HAD19 | |
| ybeX | Heat shock protein, putative Co2+ and Mg2+ efflux | Beneficial for HMDA |
| | protein; contains two CBS domains | |
| mpl | UDP-N-acetylmuramate: L-alanyl-gamma-D- | |
| | glutamyl-meso-diaminopimelate ligase; recycles cell | |
| | wall peptidoglycan | |

*K-12 strains have a frameshift mutation in rph that decreases transcription of pyrE leading to known growth defect in minimal media

g) Investigation of Causative Point Mutations

It was desired to investigate which coding mutations were also causative in a selection of the best-performing strains. Two putrescine-evolved isolates, PUTR3-1 and PUTR8-10, and two HMDA-evolved isolates, HMDA1-10 and HMDA7-1, were selected for performing conjugation-mediated genome shuffling with the wild-type background strain K-12 MG1655. This technique generates a library of mutants that undergo random transfers and recombination of segments of the genome of the evolved strains, allowing the possibility of isolating strains with only some portion of the original set of mutations that are also tolerant. One drawback of this technique is that mutations that are close to each other in the genome are frequently transferred together, and it can be difficult to effectively isolate mutants that underwent multiple conjugation events to transfer the required mutations.

Selected isolates that were obtained as described in the methods were grown in the Biolector with 38 g/L putrescine or HMDA and were whole-genome sequenced. The mutations present in each isolate (e.g. PUTR3-1_1) are annotated in the plots. New mutations that were not present in the evolved isolate are indicated in red, while mutations that were present in the original evolved isolate are shown in black (with the full genotype of the evolved isolate also displayed). It was decided to not resequence any isolates following conjugation with HMDA1-10, as growth screening revealed no isolates with a tolerance phenotype approaching that of HMDA1-10.

For PUTR3-1 (Table 24), most resequenced conjugants exhibit growth approaching the evolved isolate PUTR3-1, and all conjugants harbor the coding mutation in ygaC (R43L). Four out of 6 also harbor the mutation in the intergenic region between edd and zwf, including isolate PUTR3-1_12, which exhibits the highest growth rates of all conjugated isolates. Based on these results, it was decided to introduce the ygaC and edd/zwf point mutations into the ΔproV background strain (see next section), due to deletion in proV already having been shown to improve growth in putrescine.

For PUTR8-10 (Table 25), it appeared to be more difficult for multiple conjugation events to occur that would transfer all necessary mutations required for the PUTR8-10 phenotype to the wild-type background. A number of conjugated isolates clustered together with their growth behavior (PUTR8-10_1, 4, 6, 9, and 12), and all of these isolates harbored more mutations from PUTR8-10 than the PUTR8-10_10 isolate. It is possible that these poorer growing isolates harbor a combination of mutations (for example, they all have the intergenic mutation between pyrE and rph) that reduces growth without the presence of every mutation in PUTR8-10. Because the #10 isolate exhibited the best growth, it was decided to introduce the mreB (A298V), rpsG (L157*), and spoT (R471H) mutations into the ΔproV background strain (see next section), due to deletion in proV already having been shown to improve growth in putrescine and because the frameshift mutation in proX, another subunit of the ProVWX ABC transporter, is extremely likely to be functionally equivalent to disrupting proV. It was also decided to reconstruct the argG non-coding point mutation.

For HMDA7-1 (Table 26), the majority of conjugated isolates exhibited growth behavior approximately equivalent to HMDA7-1. All strains exhibited a common core set of 3 mutations in nusA (L152R), sspA (F83C), and rpsG (L157*). As a result, it was decided to introduce these three mutations into the ΔproV background strain (see next section), due to deletion in proV already having been shown to improve growth in HMDA.

TABLE 24
Biolector growth screen and detected variants following resequencing in K-12 MG1655 isolates
that had been conjugated with strain PUTR3-1 and selected for growth on 38 g/L putrescine

| | | mean (1) | | std. error (1) | | mean (2) | | std. error (2) | |
|---|---|---|---|---|---|---|---|---|---|
| strain | mutations | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | — | 0.205 | 16.2 | 0.105 | 0.6 | 0.000 | — | 0.000 | — |
| PUTR3-1 | mcrA/icdC G→T, | 0.393 | 12.1 | 0.029 | 0.6 | 0.343 | 13.8 | 0.009 | 0.3 |
| | edd/zwf C→T, ygaC | | | | | | | | |
| | R43L, proV 1 bp ins., | | | | | | | | |
| | rph/yicC C→A, spoT | | | | | | | | |
| | R209H, nusG G166V, | | | | | | | | |
| | lexN N163I | | | | | | | | |
| PUTR3-1_1 | edd/zwf C→T, fliK 9 bp | 0.282 | 11.4 | 0.003 | 0.4 | 0.206 | 10.1 | 0.005 | 0.4 |
| | amp., ygaC R43L, proV | | | | | | | | |
| | 1 bp ins. | | | | | | | | |
| PUTR3-1_3 | iscR L113F, ygaC R43L, | 0.280 | 11.7 | 0.016 | 0.3 | 0.221 | 10.3 | 0.004 | 0.4 |
| | proV 1 bp ins. | | | | | | | | |
| PUTR3-1_7 | edd/zwf C→T, ygaC | 0.243 | 11.9 | 0.001 | 0.2 | 0.201 | 10.8 | 0.008 | 0.1 |
| | R43L, proV 1 bp ins. | | | | | | | | |
| PUTR3-l_10 | edd/zwf C→T, fliK 9 bp | 0.280 | 11.1 | 0.002 | 0.2 | 0.241 | 12.2 | 0.020 | 0.4 |
| | amp., iscR L113F, ygaC | | | | | | | | |
| | R43L, proV 1 bp ins. | | | | | | | | |
| PUTR3-1_12 | edd/zwf C→T, ygaC | 0.196 | 11.7 | 0.011 | 0.2 | 0.234 | 9.6 | 0.006 | 0.5 |
| | R43L | | | | | | | | |
| PUTR3-1_13 | not resequenced | 0.250 | 12.0 | 0.017 | 0.6 | 0.133 | 8.4 | 0.007 | 0.4 |
| PUTR3-1_15 | fliK 9 bp amp., iscR | 0.248 | 11.9 | 0.010 | 0.8 | 0.191 | 9.2 | 0.027 | 0.9 |
| | L113F, ygaC R43L, proV | | | | | | | | |
| | 1 bp ins. | | | | | | | | |

TABLE 25
Biolector growth screen and detected variants following resequencing in K-12 MG1655 isolates
that had been conjugated with strain PUTR8-10 and selected for growth on 38 g/L putrescine

| | | mean (1) | | std. error (1) | | mean (2) | | std. error (2) | |
|---|---|---|---|---|---|---|---|---|---|
| strain | mutations | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | none | 0.205 | 16.2 | 0.105 | 0.6 | 0.000 | — | 0.000 | 0.6 |
| PUTR8-10 | sfmH F11S, nagC | 0.363 | 4.5 | 0.035 | 0.9 | 0.347 | 7.8 | 0.016 | 0.2 |
| | 47 bp del., proX 1 | | | | | | | | |
| | bp ins., argG | | | | | | | | |
| | C→A, mreB | | | | | | | | |
| | A298V, rpsG | | | | | | | | |
| | L157*, pyrE/rph | | | | | | | | |
| | 1 bp del., spoT | | | | | | | | |
| | R471H | | | | | | | | |
| PUTR8-10_1 | proX 1 bp ins., | 0.218 | 11.3 | 0.016 | 1.7 | 0.192 | 9.4 | 0.022 | 0.4 |
| | argG C→A, mreB | | | | | | | | |
| | A298V, rpsG | | | | | | | | |
| | L157*, pyrE/rph | | | | | | | | |
| | 1 bp del., spoT | | | | | | | | |
| | R471H | | | | | | | | |
| PUTR8-10_4 | argG C→A, mreB | 0.202 | 9.7 | 0.092 | 6.6 | 0.206 | 11.4 | 0.028 | 0.2 |
| | A298V, rpsG | | | | | | | | |
| | L157*, pyrE/rph | | | | | | | | |
| | 1 bp del., spoT | | | | | | | | |
| | R471H | | | | | | | | |
| PUTR8-10_6 | argG C→A, mreB | 0.216 | 11.6 | 0.012 | 2.9 | 0.180 | 15.5 | 0.066 | 11.4 |
| | A298V, rpsG | | | | | | | | |
| | L157*, pyrE/rph | | | | | | | | |
| | 1 bp del., spoT | | | | | | | | |
| | R471H | | | | | | | | |
| PUTR8-10_9 | proX 1 bp ins., | 0.211 | 7.6 | 0.018 | 0.0 | 0.201 | 11.4 | 0.025 | 0.3 |
| | argG C→A, mreB | | | | | | | | |
| | A298V, rpsG | | | | | | | | |
| | L157*, pyrE/rph | | | | | | | | |
| | 1 bp del., spoT | | | | | | | | |
| | R471H | | | | | | | | |
| PUTR8-10_10 | proX 1 bp ins., | 0.271 | 6.6 | 0.013 | 0.6 | 0.198 | 10.4 | 0.011 | 0.3 |
| | mreB A298V, | | | | | | | | |
| | rpsG L157*, spoT | | | | | | | | |
| | R471H | | | | | | | | |
| PUTR8-10_12 | proX 1 bp ins., | 0.243 | 8.9 | 0.025 | 0.9 | 0.270 | 9.7 | 0.027 | 0.9 |
| | argG C→A, mreB | | | | | | | | |
| | A298V, rpsG | | | | | | | | |
| | L157*, pyrE/rph | | | | | | | | |
| | 1 bp del., spoT | | | | | | | | |
| | R471H | | | | | | | | |

TABLE 26
Biolector growth screen and detected variants following resequencing in K-12 MG1655 isolates
that had been conjugated with strain HMDA7-1 and selected for growth on 38 g/L HMDA

| | | mean (1) | | std. error (1) | | mean (2) | | std. error (2) | |
|---|---|---|---|---|---|---|---|---|---|
| strain | mutations | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | none | 0.000 | — | 0.000 | — | 0.000 | — | 0.000 | — |
| HMDA7-1 | wbbK 1 bp del., | 0.221 | 2.7 | 0.045 | 1.0 | 0.254 | 7.1 | 0.070 | 0.6 |
| | argY/argV 284 bp | | | | | | | | |
| | del., nusA L152R, | | | | | | | | |
| | sspA F83C, rpsG | | | | | | | | |
| | L157* | | | | | | | | |
| HMDA7-1_4 | wbbK 1 bp del., | 0.189 | 2.7 | 0.066 | 0.5 | 0.167 | 2.4 | 0.035 | 3.3 |
| | rfbB D58Y, | | | | | | | | |
| | argY/argV 284 bp | | | | | | | | |
| | del., nusA L152R, | | | | | | | | |
| | sspA F83C, rpsG | | | | | | | | |
| | L157* | | | | | | | | |
| HMDA7-1_5 | nusA L152R, sspA | 0.268 | 3.3 | 0.065 | 0.7 | 0.234 | 5.8 | 0.030 | 0.2 |
| | F83C, rpsG L157* | | | | | | | | |
| HMDA7-1_7 | argY/argV 284 bp | 0.278 | 2.9 | 0.022 | 0.7 | 0.265 | 5.4 | 0.012 | 0.9 |
| | del., nusA L152R, | | | | | | | | |
| | sspA F83C, rpsG | | | | | | | | |
| | L157* | | | | | | | | |
| HMDA7-1_8 | nusA L152R, sspA | 0.245 | 2.2 | 0.072 | 1.2 | 0.151 | 3.4 | 0.024 | 0.7 |
| | F83C, rpsG L157* | | | | | | | | |
| HMDA7-1_11 | argY/argV 284 bp | 0.266 | 2.8 | 0.034 | 0.9 | 0.195 | 1.9 | 0.023 | 0.2 |
| | del., nusA L152R, | | | | | | | | |
| | sspA F83C, rpsG | | | | | | | | |
| | L157* | | | | | | | | |
| HMDA7-1_13 | argY/argV 284 bp | 0.286 | 3.2 | 0.048 | 1.1 | 0.226 | 5.7 | 0.016 | 2.0 |
| | del., nusA L152R, | | | | | | | | |
| | sspA F83C, rpsG | | | | | | | | |
| | L157*, yjiJ IS150 | | | | | | | | |
| | ins. | | | | | | | | |

h) Reconstruction and Testing of Mutants Harboring Point Mutations

For PUTR3-1, the ygaC (R43L) and intergenic edd/zwf mutations were first introduced individually into K-12 MG1655 ΔproV, and next the combination of the two mutations was made also in the ΔproV background. These mutants were tested against K-12 MG1655 and PUTR3-1 in a growth screen in the Biolector testing format with 38 g/L putrescine (Table 27). It was evident that the edd/zwf mutation exhibited no discernible phenotype, while the ygaC and ygaC plus edd/zwf mutant strains exhibited an identical phenotype, thus we could assign the ygaC (R43L) mutation as causative and responsible for the majority of the growth improvement in this strain in high concentrations of putrescine. The growth rate is dramatically improved in this mutant over the K-12 MG1655 background but it is still a little lower than the original PUTR3-1 evolved strain. The ygaC mutation has also been constructed in K-12 MG1655.

For PUTR8-10, the rpsG (L157*), argG (non-coding), mreB (A298V), and spoT (R471H) mutations were first introduced individually into K-12 MG1655 ΔproV (and the rpsG mutation was also introduced individually into K-12 MG1655), and next the double combinations of the mreB, spoT, and argG mutations with the rpsG mutation were constructed. These mutants were tested against K-12 MG1655 and PUTR8-10 as described for PUTR3-1 (Table 27). Mutants harboring the spoT mutation by itself and in combination with the rpsG mutation could not grow in 38 g/L putrescine. Thus we can conclude that this mutation needs to be present together with other mutations in PUTR8-10 to either have a neutral or positive growth benefit. The argG mutation by itself afforded a moderately improved growth rate increase, while the rpsG and mreB afforded dramatically improved growth rates when present individually. For rpsG mutants, growth rate was equivalently improved in both the K-12 MG1655 and ΔproV background strains, indicating that disruption of proV afforded no additional growth advantage in the presence of these mutations (also suggested by the conjugated isolate results in the previous section). Both the argG and mreB double mutants with rpsG exhibited further improved growth characteristics, with the rpsG and mreB double combination being the best tested to date, with a growth rate and lag time approaching that of PUTR8-10. The triple combination of the rpsG, mreB, and argG mutations is being constructed and will be tested in the near future. It is believed that the continued growth of PUTR8-10 where the other mutants enter stationary phase may be a result of the spoT mutation, which encodes an enzyme that both synthesizes and hydrolyzes (p)ppGpp, an molecule that binds RNA polymerase and signals cells to undergo the stringent response. An impairing of the stringent response in PUTR8-10 would explain its continued growth when other cells stop growing and enter the stationary phase.

For HMDA7-1, the nusA (L152R), sspA (F83C) were attempted to be introduced individually into K-12 MG1655 ΔproV. The rpsG (L157*) mutant had already been constructed for investigating PUTR8-10 in both K-12 MG1655 and the ΔproV background. While the sspA (F83C) mutant could be readily constructed, it was not possible to isolate a nusA (L152R) mutant out of over 100 screened colonies. With a significant screening effort, it was possible to isolate the nusA mutant in the strain already harboring the sspA mutant. Thus this mutation alone is likely greatly reducing fitness during MAGE and subsequent plating, which is performed using LB medium. Thus the sspA and rpsG single mutants (both in K-12 MG1655 and the ΔproV background strain) and sspA nusA double mutant in K-12 MG1655 ΔproV were tested for growth in the Biolector in 38 g/L HMDA (Table 28). Both the sspA mutant and the rpsG mutants exhibited greatly improved growth, with the ΔproV mutation affording a negligible additional growth benefit. The nusA and sspA double mutant strain exhibited dramatically improved growth over the sspA single mutant. Additional combinations with the rpsG mutation are currently being constructed and will be tested in the near future. A nusA sspA rpsG triple mutant which was validated to have received the rpsG mutation was found by Sanger sequencing to have an additional mutated base in the nusA locus, highlighting the instability of the nusA (L152R) mutation and probable negative fitness cost in LB medium.

A summary of point mutant strains that improve tolerance to putrescine and HMDA is provided in Table 29. Without being limited to theory, these are believed to not be complete losses-of-function. The strains that did not exhibit improved growth are also shown in Tables 27 and 28. Descriptions of the gene names and functions are provided in Table 29.

TABLE 27
Growth of tested point mutant strains in high concentrations of putrescine

| | mean (1) | | std. error (1) | | mean (2) | | std. error (2) | |
|---|---|---|---|---|---|---|---|---|
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.074 | 21.8 | 0.011 | 4.6 | 0.000 | — | 0.000 | — |
| MG1655 proV::kan | 0.094 | 18.2 | 0.026 | 0.8 | 0.091 | 14.7 | 0.015 | 9.0 |
| PUTR3-1 | 0.317 | 11.3 | 0.021 | 0.5 | 0.345 | 13.4 | 0.013 | 0.8 |
| MG1655 ΔproV ygaC* | 0.221 | 12.7 | 0.017 | 0.3 | 0.251 | 12.9 | 0.005 | 0.8 |
| MG1655 ΔproV edd/zwf* | 0.106 | 16.3 | 0.014 | 4.9 | 0.100 | 14.4 | 0.022 | 0.1 |
| MG1655 ΔproV ygaC* edd/zwf* | 0.217 | 12.8 | 0.010 | 1.4 | 0.238 | 13.2 | 0.021 | 0.5 |
| PUTR8-10 | 0.274 | 2.7 | 0.015 | 0.9 | 0.318 | 7.8 | 0.010 | 0.5 |
| MG1655 rpsG* | 0.253 | 2.7 | 0.032 | 4.2 | 0.230 | 9.8 | 0.013 | 0.7 |
| MG1655 ΔproV rpsG* | 0.216 | 3.2 | 0.014 | 1.3 | 0.182 | 7.4 | 0.021 | 1.4 |
| MG1655 ΔproV mreB* | 0.266 | 11.5 | 0.010 | 0.1 | 0.270 | 11.9 | 0.009 | 0.3 |
| MG1655 ΔproV spoT* | 0.000 | — | 0.000 | — | 0.000 | — | 0.000 | — |
| MG1655 ΔproV argG* | 0.132 | 13.4 | 0.018 | 3.7 | 0.124 | 13.5 | 0.033 | 2.6 |
| MG1655 ΔproV rpsG* mreB* | 0.235 | 6.4 | 0.006 | 1.9 | 0.267 | 8.0 | 0.004 | 0.7 |
| MG1655 ΔproV rpsG* spoT* | 0.119 | 2.0 | 0.007 | 3.2 | 0.094 | 8.4 | 0.022 | 5.2 |
| MG1655 ΔproV rpsG* argG* | 0.201 | 3.4 | 0.024 | 5.0 | 0.258 | 10.3 | 0.005 | 1.5 |

TABLE 28
Growth of tested point mutant strains in high concentrations of HMDA

| | mean (1) | | std. error (1) | | mean (2) | | std. error (2) | |
|---|---|---|---|---|---|---|---|---|
| strain | μ (h⁻¹) | t_{lag} (h) | μ (h⁻¹) | t_{lag} (h) | μ (h⁻¹) | t_{lag} (h) | μ (h⁻¹) | t_{lag} (h) |
| MG1655 | 0.511 | 31.6 | 0.049 | 6.2 | 0.664 | 32.8 | 0.074 | 5.9 |
| MG1655 proV::kan | 0.453 | 26.1 | 0.098 | 4.0 | 0.605 | 27.8 | 0.155 | 4.2 |
| HMDA7-1 | 0.541 | 4.7 | 0.074 | 0.3 | 0.387 | 4.5 | 0.017 | 0.8 |
| MG1655 ΔproV rpsG* | 0.276 | 8.2 | 0.016 | 1.1 | 0.271 | 12.0 | 0.001 | 1.6 |
| MG1655 ΔproV sspA* | 0.207 | 11.4 | 0.022 | 8.9 | 0.271 | 18.5 | 0.008 | 0.7 |
| MG1655 ΔproV sspA* nusA* | 0.316 | 6.5 | 0.009 | 4.5 | 0.289 | 9.7 | 0.007 | 1.2 |
| MG1655 rpsG* | 0.252 | 6.2 | 0.030 | 2.2 | 0.225 | 9.1 | 0.001 | 1.2 |

TABLE 29
Descriptions of genes disrupted in mutants with improved
growth in high concentrations of putrescine and HMDA

| Gene | | |
|---|---|---|
| name | Description | Notes |
| ygaC | Function unknown; Fur regulon | |
| rpsG | 30S ribosomal subunit protein S7, | see note below* |
| | mutated stop codon | |
| argG | Argininosuccinate synthase | the last enzyme of arginine |
| | | biosynthesis; arginine is an |
| | | intracellular precursor for |
| | | native putrescine production |
| mreB | Cell wall structural actin-like | see note below** |
| | protein in MreBCD complex; | |
| | mecillinam resistance protein | |
| sspA | DUF2527 family heat-induced | In the same operon as cspC |
| | protein, function unknown | |
| nusA | N-acetylglucosamine-inducible nag | In the same operon as nagA |
| | divergent operon transcriptional | |
| | repressor | |

*K-12 MG1655 has a mutation in rpsG relative to other E. coli strains that results in a lengthened ORF; evolution in various stress conditions appears to result in retruncation (via a premature stop codon). The neighboring W156* mutation has been observed in strains evolved to Na⁺ (Wu et al., Appl. Environ. Microbiol., 80: 2880-2888, 2014) and in one of our p-coumarate evolved populations (COUM4). We have shown that for p-coumarate, the mutation is strongly selected for in the presence of p-coumarate but not in M9 glucose medium.
**Different coding mutations in MreB have previously been observed during evolutions on high NaCl concentrations (I336L, T171S, S185F, K96Q; Winkler et al., Appl. Environ. Microbiol., 80: 3729-3740, 2014). Other MreB mutations that we have isolated from putrescine evolutions are N34K, E212A. I24M, and H93N. None occurred in our HMDA evolved isolates.

i) Reconstruction of ybeX and Mpl Knockouts in Existing Most Tolerant Strains

The Keio collection screening hits in HMDA, ybeX and mpl, were constructed in K-12 MG1655 as single knockouts. Additionally, single ybeX or mpl knockouts or the combination of both the ybeX and mpl knockouts were constructed in K-12 MG1655 harboring the single rpsG (L157*) mutation, the rpsG (L157*) and mreB (A298V) mutations, the ygaC (R43L) mutation, the nusA (L152R) and sspA (F83C) mutations, and in the proV cspC and proV ptsP double knockout strains. All of these strains were tested in the Biolector growth testing format in M9+38 g/L putrescine and M9+38 g/L HMDA.

In 38 g/L putrescine (Table 30 and Table 31), it is apparent that the ybeX mutation does not improve growth by itself, while the mpl single knockout strain exhibits a moderately improved growth rate and greatly reduced lag time. The ybeX mutation similarly reduces or results in unchanged growth relative to the background controls when in combination with other beneficial mutations. The mpl mutation uniformly improves growth in combination with other beneficial mutations, with the exception of the rpsG (L157*) mreB (A298V) strain, where the growth rate was unchanged. It should also be noted that the nusA (L152R) and sspA (F83C) mutations, in addition to the evolved strain HMDA7-1, exhibit improved growth in 38 g/L putrescine in addition to HMDA, illustrating the cross-resistance of these strains and mutants across inhibitory concentrations of different polyamines in most cases.

In 38 g/L HMDA (Table 32 and Table 33), the ybeX mutation significantly improves growth by itself. The mpl single knockout also exhibits improved growth, although to a lesser extent than the ybeX knockout strain. Both the ybeX and mpl knockouts additively improve growth rates and lag times in background strains, and the combination of both the ybeX and mpl knockouts generally further improves growth over the single knockouts in either gene. It should also be noted that the evolved strains PUTR3-1 and PUTR8-10 and other reconstructed strains that were originally only tested in 38 g/L putrescine, also exhibit greatly improved growth in 38 g/L HMDA. In particular, strains K-12 MG1655 ΔproV nusA (L152R) sspA (F83C) ΔybeX mpl::kan and K-12 MG1655 ΔproV rpsG (L157*) mreB (A298V) LybeX mpl::kan exhibited similar growth rates and lag times to the evolved isolate HMDA7-1, illustrating a full reconstruction of the tolerance phenotype in evolved isolates via a combination of additive mutations from multiple isolates.

TABLE 30
Growth rates and lag times of selected knockout/MAGE mutants in M9 +
38 g/L putrescine, as measured in the Biolector testing format.

| | mean (2) | | std. error (2) | |
|---|---|---|---|---|
| | μ | t_{lag} | μ | t_{lag} |
| strain | (h⁻¹) | (h) | (h⁻¹) | (h) |
| MG1655 | 0.075 | 23.8 | 0.018 | 1.7 |
| HMDA7-1 | 0.204 | 7.4 | 0.016 | 0.9 |
| MG1655 ΔproV rpsG* | 0.234 | 9.4 | 0.010 | 0.7 |
| MG1655 ΔproV nusA* sspA* | 0.095 | 0.0 | 0.009 | 0.0 |
| MG1655 ybeX::kan | 0.058 | 12.2 | 0.017 | 10.7 |
| MG1655 mpl::kan | 0.116 | 7.5 | 0.004 | 0.8 |
| MG1655 ΔproV rpsG* ybeX::kan | 0.182 | 10.9 | 0.006 | 0.8 |
| MG1655 ΔproV rpsG* mpl::kan | 0.196 | 5.8 | 0.009 | 0.9 |
| MG1655 ΔproV rpsG* ΔybeX mpl::kan | 0.139 | 4.4 | 0.006 | 1.4 |
| MG1655 ΔproV nusA* sspA* ybeX::kan | 0.088 | 0.0 | 0.020 | 0.0 |
| MG1655 ΔproV nusA* sspA* mpl::kan | 0.138 | 1.1 | 0.029 | 1.6 |
| MG1655 ΔproV nusA* sspA* ΔybeX | 0.144 | 4.4 | 0.028 | 3.3 |
| mpl::kan | | | | |
| MG1655 ΔproV ptsP::kan | 0.090 | 30.9 | 0.031 | 0.7 |
| MG1655 ΔproV ΔptsP ybeX::kan | 0.064 | 33.1 | 0.036 | 23.2 |
| MG1655 ΔproV ΔptsP mpl::kan | 0.133 | 18.2 | 0.019 | 1.3 |
| MG1655 ΔproV ΔptsP ΔybeX mpl::kan | 0.086 | 18.5 | 0.006 | 3.5 |

TABLE 31
Growth rates and lag times of selected knockout/MAGE mutants in M9 +
38 g/L putrescine, as measured in the Biolector testing format.

| | mean (2) | | std. error (2) | |
|---|---|---|---|---|
| | μ | t_{lag} | μ | t_{lag} |
| strain | (h⁻¹) | (h) | (h⁻¹) | (h) |
| MG1655 | 0.081 | 25.1 | 0.053 | 10.3 |
| PUTR3-1 | 0.339 | 13.6 | 0.013 | 0.9 |
| MG1655 ΔproV ygaC* | 0.229 | 12.8 | 0.021 | 0.2 |
| MG1655 ΔproV ygaC* ybeX::kan | 0.152 | 13.4 | 0.022 | 0.9 |
| MG1655 ΔproV ygaC* mpl::kan | 0.224 | 11.8 | 0.011 | 0.4 |
| MG1655 ΔproV ygaC* ΔybeX mpl::kan | 0.129 | 11.8 | 0.006 | 0.5 |
| PUTR8-10 | 0.332 | 8.6 | 0.006 | 0.2 |
| MG1655 ΔproV rpsG* mreB* | 0.280 | 7.7 | 0.004 | 0.6 |
| MG1655 ΔproV rpsG* mreB* ybeX::kan | 0.269 | 10.0 | 0.010 | 0.6 |
| MG1655 ΔproV rpsG* mreB* mpl::kan | 0.273 | 9.5 | 0.005 | 0.3 |
| MG1655 ΔproV rpsG* mreB* ΔybeX | 0.242 | 10.0 | 0.009 | 0.9 |
| mpl::kan | | | | |
| MG1655 ΔproV cspC::kan | 0.159 | 28.3 | 0.046 | 2.9 |
| MG1655 ΔproV ΔcspC ybeX::kan | 0.155 | 27.7 | 0.049 | 0.6 |
| MG1655 ΔproV ΔcspC mpl::kan | 0.192 | 17.7 | 0.017 | 1.7 |
| MG1655 ΔproV ΔcspC ΔybeX mpl::kan | 0.186 | 18.4 | 0.017 | 1.3 |

TABLE 32
Growth rates and lag times of selected knockout/MAGE mutants in
M9 + 38 g/L HMDA, as measured in the Biolector testing format.

| | mean (2) | | std. error (2) | |
|---|---|---|---|---|
| | μ | t_{lag} | μ | t_{lag} |
| strain | (h⁻¹) | (h) | (h⁻¹) | (h) |
| MG1655 | 0.092 | 42.7 | 0.034 | 1.9 |
| HMDA7-1 | 0.419 | 6.2 | 0.007 | 0.6 |
| MG1655 ΔproV rpsG* | 0.249 | 12.3 | 0.007 | 0.7 |
| MG1655 ΔproV nusA* sspA* | 0.317 | 14.9 | 0.064 | 10.6 |
| MG1655 ybeX::kan | 0.171 | 6.2 | 0.005 | 0.9 |
| MG1655 mpl::kan | 0.131 | 23.6 | 0.010 | 0.5 |
| MG1655 ΔproV rpsG* ybeX::kan | 0.280 | 6.9 | 0.009 | 0.3 |
| MG1655 ΔproV rpsG* mpl::kan | 0.287 | 8.9 | 0.020 | 0.6 |
| MG1655 ΔproV rpsG* ΔybeX mpl::kan | 0.315 | 6.6 | 0.007 | 0.0 |
| MG1655 ΔproV nusA* sspA* ybeX::kan | 0.292 | 5.5 | 0.031 | 1.5 |
| MG1655 ΔproV nusA* sspA* mpl::kan | 0.341 | 11.9 | 0.027 | 0.7 |
| MG1655 ΔproV nusA* sspA* ΔybeX | 0.410 | 5.6 | 0.037 | 0.6 |
| mpl::kan | | | | |
| MG1655 ΔproV ptsP::kan | 0.139 | 30.3 | 0.067 | 18.0 |
| MG1655 ΔproV ΔptsP ybeX::kan | 0.243 | 19.1 | 0.031 | 2.9 |
| MG1655 ΔproV ΔptsP mpl::kan | 0.306 | 23.8 | 0.003 | 2.8 |
| MG1655 ΔproV ΔptsP ΔybeX mpl::kan | 0.340 | 16.2 | 0.012 | 2.3 |

TABLE 33
Growth rates and lag times of selected knockout/MAGE mutants in
M9 + 38 g/L HMDA, as measured in the Biolector testing format.

| | mean (2) | | std. error (2) | |
|---|---|---|---|---|
| | μ | t_{lag} | μ | t_{lag} |
| strain | (h⁻¹) | (h) | (h⁻¹) | (h) |
| MG1655 | 0.108 | 29.3 | 0.003 | 2.7 |
| PUTR3-1 | 0.461 | 21.0 | 0.006 | 0.7 |
| MG1655 ΔproV ygaC* | 0.256 | 18.6 | 0.005 | 1.1 |
| MG1655 ΔproV ygaC* ybeX::kan | 0.391 | 9.2 | 0.009 | 0.3 |
| MG1655 ΔproV ygaC* mpl::kan | 0.260 | 14.0 | 0.009 | 0.9 |
| MG1655 ΔproV ygaC* ΔybeX mpl::kan | 0.356 | 10.8 | 0.048 | 1.4 |
| PUTR8-10 | 0.402 | 13.4 | 0.012 | 0.7 |
| MG1655 ΔproV rpsG* mreB* | 0.354 | 9.9 | 0.004 | 0.6 |
| MG1655 ΔproV rpsG* mreB* ybeX::kan | 0.414 | 8.5 | 0.005 | 0.5 |
| MG1655 ΔproV rpsG* mreB* mpl::kan | 0.324 | 10.5 | 0.000 | 0.1 |
| MG1655 ΔproV rpsG* mreB* ΔybeX | 0.405 | 8.7 | 0.020 | 0.5 |
| mpl::kan | | | | |
| MG1655 ΔproV cspC::kan | 0.191 | 40.8 | 0.032 | 3.0 |
| MG1655 ΔproV ΔcspC ybeX::kan | 0.302 | 12.9 | 0.003 | 1.9 |
| MG1655 ΔproV ΔcspC mpl::kan | 0.258 | 26.4 | 0.002 | 3.4 |
| MG1655 ΔproV ΔcspC ΔybeX mpl::kan | 0.360 | 13.5 | 0.012 | 1.5 |

j) Flow Cytometric Analysis of Cell Morphology

Cell morphological changes are suspected in many putrescine evolved strains due to the common occurrence of mutations in MreB and other cell wall related genes (e.g. MrdB, MurA, McrA). MreB has a well-known role in forming cytosolic protein filaments that interact with the inner membrane, assisting in the maintenance of cylindrical cell shape. Mutations that disrupt mreB have most commonly been observed to result in spherical cells and often cell lysis. Other genes are more directly related to peptidoglycan synthesis and maintenance. Cell morphology in a population can be analyzed in a quantitative manner through the measurement of forward and side scattered light. Forward scatter is related to cell size, with higher forward scatter intensities correlated with larger cell dimensions. Side scatter is related to the cell refractive index, with increased side scatter intensities correlating with a higher order of internal complexity (for example, curvature of membrane structures). In ordinary wild-type cells, cell shape varies as a function of the phase of growth. Exponentially growing cells are typically longer and more cylindrical. Stationary phase cells are typically smaller and more spherical.

A preliminary analysis of PUTR3-1 (no known cell wall related mutations present), PUTR4-3 (coding mutations in MrdA), and PUTR8-10 (MreB A298V) in stationary phase cells grown in M9+1% glucose indicated a larger average cell size in PUTR8-10 and a smaller average cell size in PUTR4-3, with PUTR3-1 having an approximately equivalent cell size to wild-type cells. This is suggestive of the MrdA and MreB coding mutations having resulted in these phenotypes.

Additional screens of all sequenced putrescine evolved strains indicate similar results for some strains, with smaller cell size in e.g. the PUTR4, PUTR7-7 and PUTR7-9, and PUTR6-2 strains in stationary phase (which all harbor mutations in either mrdA and murA) and larger cell size in exponential phase than wild-type cells.

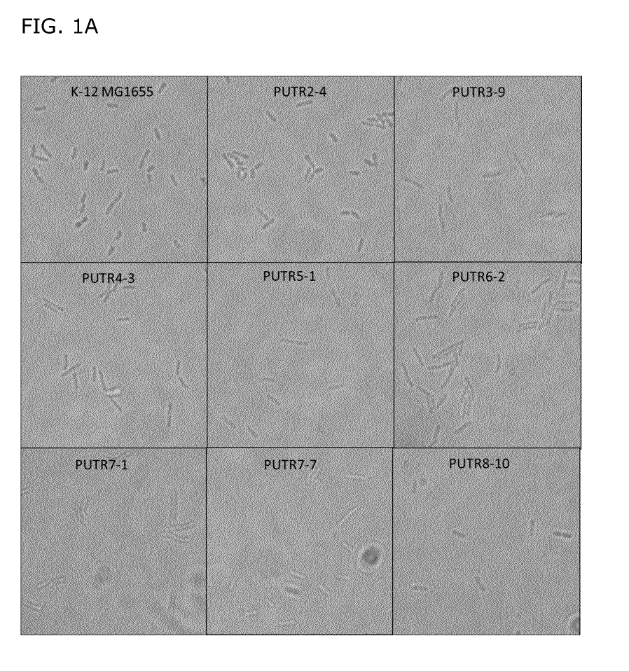

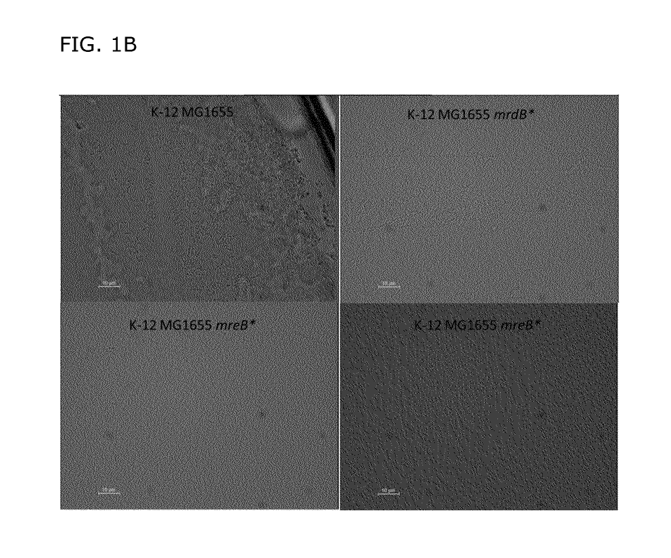

A flow cytometric screen was conducted for putrescine and HMDA-evolved isolates that were identified to have mutations in genes related to the cell wall or maintenance of cell shape. Forward scatter intensities are non-linearly correlated with cell size, and are shown for each isolate in Table 34. PUTR3-9, PUTR4-3, PUTR5-1, PUTR6-2, PUTR7-1, and PUTR7-7 all exhibited reduced exponential phase (in M9 medium) forward scatter intensities (FSC) as compared with K-12 MG1655. These isolates were also analyzed by phase contrast microscopy during exponential phase in M9 medium and it was found that all strains with reduced FSC values exhibited a more elongated and narrower cell shape (FIG. 1).

The MrdB-E254K mutation was introduced by MAGE into K-12 MG1655, and this strain was grown in 38 g/L putrescine in the Biolector testing format (Table 35). While it is difficult to capture the greatly improved growth profile observed in this strain in this table, it is apparent that the lag time was greatly improved. The strain also grew to a density nearly equivalent to PUTR4-3, whereas K-12 MG1655 remained at a very low cell density. Thus this mutation appeared to be one of the most causative mutations in the PUTR4-3 background. It was also apparent by phase contrast microscopy that the MrdB-E254K mutation alone reconstitutes the cell morphology found in PUTR4-3, demonstrating that the identified cell wall mutations are likely responsible for the morphological phenotypes observed in other isolates.

TABLE 34
Forward scatter intensity of selected putrescine and HMDA
evolved isolates containing cell wall or cell shape related
mutations indicated, as measured by flow cytometry of
exponential phase cultures grown in M9 medium.

FSC intensity

| strain | cell wall mutation | average | std. error |
|---|---|---|---|
| MG1655 | — | 433.3 | 11.6 |
| PUTR2-4 | MreB-N34K | 521.0 | 9.0 |
| PUTR3-9 | MreB-E212A | 316.3 | 20.5 |
| PUTR4-3 | MrdB-E254K | 322.7 | 4.2 |
| PUTR5-1 | MreB-I24M | 339.7 | 9.2 |
| PUTR6-2 | MurA-G141A | 330.0 | 19.7 |
| PUTR7-1 | MreB-H93N | 342.7 | 7.8 |
| PUTR7-7 | MurA-Y393S | 340.3 | 28.6 |
| PUTR8-10 | MreB-A298V | 482.7 | 12.5 |
| HMDA5-4 | AmpC-I205T | 466.7 | 3.2 |

TABLE 35
Growth rates and lag times of K-12 MG1655, PUTR4-3, and K-12
MG1655 harboring the MrdA-E254K mutation in M9 + 38
g/L putrescine, as measured in the Biolector testing format.

| | |
|---|---|
| mean (2) | std. error (2) |

| | μ | t_{lag} | μ | t_{lag} |
|---|---|---|---|---|
| strain | (h⁻¹) | (h) | (h⁻¹) | (h) |
| MG1655 | 0.153 | 18.9 | 0.008 | 0.3 |
| PUTR4-3 | 0.273 | 14.2 | 0.005 | 0.8 |
| MG1655 mrdB* | 0.151 | 9.2 | 0.013 | 0.9 |

k) Cross-Compound Tolerance Testing

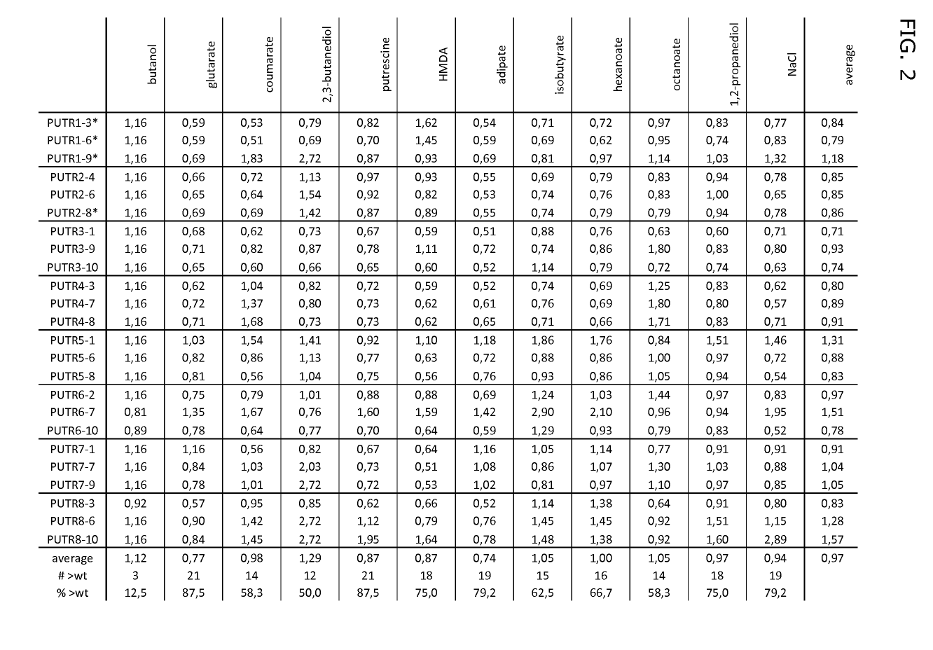

Every secondary screened evolved isolate from the putrescine and HMDA evolutions was grown in the presence of every other compound in the study as indicated in the Methods. The normalized tOD1(evolved strain)/tOD1(wild-type) are presented in FIGS. 2 and 3 for the putrescine and HDMA evolved isolates, respectively. Lower values are indicative of a larger improvement in growth of the evolved isolate (left column) in that chemical condition (top row), whereas higher values are indicative of a lower improvement or decrease in growth compared to the wild-type. Averaged ratios across conditions and strains shown at the right and bottom of the plot allow for overall by-chemical and by-strain trends to be observed. Strain names that are followed by an asterisk (*) were not re-sequenced, and strain names in italics were found to be hypermutator strains.

Generally, a wide range of patterns can be observed for growth of putrescine-evolved isolates in the different chemicals (see the Table in FIG. 2). As expected, most strains (21 out of 24) exhibit improved growth in putrescine (although it should be noted that the growth improvements will be diminished at this reduced concentration of 32 g/L). Additionally, 21 evolved isolates exhibit improved growth in 40 g/L glutarate, 18 exhibit improved growth in 32 g/L HMDA, 19 exhibit improved growth in adipate, 18 exhibit improved growth in 1,2-propanediol, and 19 exhibit improved growth in NaCl. These conditions are the majority of the chemicals present at high concentrations (30-60 g/L), therefore we can conclude that many of the mutations in PUTR strains are more generally improving tolerance to osmotic stress. Many mutations in PUTR strains appear to be maladaptive for growth in the presence of butanol and 2,3-butanediol, in particular. PUTR6-7 was a notable exception, exhibiting improved growth in butanol and 2,3-butanediol and reduced growth in the majority of high osmolarity conditions. Overall, re-sequenced strains PUTR5-1, PUTR6-7, PUTR8-6, and PUTR8-10 exhibited the poorest cross-compound tolerance.

Generally similar patterns can be observed for HMDA-evolved isolates, with 18 (out of 24) strains exhibiting improved growth in glutarate, 21 with improved growth in putrescine, and all 24 with improved growth in adipate (FIG. 3). A notable exception is that many HMDA strains exhibited poor growth in 1,2-propanediol and NaCl, and 21 strains exhibited greatly improved growth in p-coumarate (particularly populations 5 through 8). A large number of strains also exhibited improved growth in isobutyrate and octanoate, suggesting the possibility that many HMDA strains possess a general tolerance mechanism towards acids. Those HMDA strains that are tolerant to, for example p-coumarate, also have improved tolerance toward isobutyrate, hexanoate, or octanoate. Genome-wide association studies may correlate particular mutations with the observed growth phenotypes.

Additionally, each evolved isolate was tested for cross-tolerance toward other polyamines and amine-containing compounds of biotechnological interest. First, K-12 MG1655 was tested in the Growth Profiler screening format for growth in the presence of a range of concentrations of each compound: 1,3-diaminopropane, 1,5-diaminopentane (cadaverine), spermidine, citrulline, ethylenediamine, carnitine, and ornithine. Variable concentrations of these compounds elicited growth inhibition in E. coli K-12 MG1655 (Table 36). Agmatine was additionally tested, but was found to be non-toxic up to 50 g/L concentration. Based on these results, a screening concentration was selected for the evolved isolates for which wild-type cells could achieve at a growth rate of 0.2-0.3 h⁻¹ (versus uninhibited growth at 0.7-0.9 h⁻¹ in M9 glucose minimal medium). These concentrations were: 35 g/L 1,3-diaminopropane, 35 g/L cadaverine, 40 g/L spermidine, 80 g/L citrulline, 18 g/L ethylenediamine, and 10 g/L ornithine. Evolved isolates of putrescine and HMDA grown in additional chain length diamines (ethylenediamine, 1,3-diaminopropane, and cadaverine) and the native triamine metabolite spermidine are shown in Tables 37 through 40. The majority of evolved isolates exhibit greatly improved growth rates and often-reduced lag times in all of these compounds compared with K-12 MG1655. A notable exception were isolates from population HMDA7, which exhibited abolished growth in 18 g/L ethylenediamine and poor growth than the majority of evolved isolates (although improved over K-12 MG1655) in 1,3-diaminopropane. The compounds citrulline and ornithine are polyamine-containing amino acids. Again, the majority of putrescine and HMDA-evolved isolates exhibited improved growth rates in the presence of inhibitory levels of these compounds for wild-type K-12 MG1655 (Tables 37 through 40). Among the only exceptions were PUTR7-1 in citrulline and PUTR5-1 and PUTR6-2 in ornithine.

TABLE 36
Growth rates and lag times of K-12 MG1655 in varying concentrations
of agmatine, ethylenediamine, carnitine, citrulline, and ornithine,
as measured in the Growth Profiler testing format.

| | μ | t_{lag} | μ | t_{lag} |
|---|---|---|---|---|
| | (h⁻¹) | (h) | (h⁻¹) | (h) |
| agmatine (g/L) | | | | |
| 0 | 0.747 | 5.1 | 0.030 | 0.2 |
| 10 | 0.700 | 5.3 | 0.039 | 0.2 |
| 20 | 0.630 | 5.6 | 0.070 | 0.1 |
| 25 | 0.648 | 6.1 | 0.101 | 0.3 |
| 30 | 0.610 | 6.3 | 0.056 | 0.2 |
| 40 | 0.632 | 7.0 | 0.046 | 0.2 |
| 50 | 0.523 | 7.9 | 0.036 | 0.2 |
| ethylenediamine (g/L) | | | | |
| 0 | 0.810 | 6.1 | 0.038 | 0.3 |
| 5 | 0.689 | 7.0 | 0.052 | 0.3 |
| 10 | 0.614 | 8.5 | 0.015 | 0.2 |
| 20 | 0.152 | 32.9 | 0.017 | 1.9 |
| 25 | 0.000 | — | 0.000 | — |
| carnitine (g/L) | | | | |
| 0 | 0.885 | 6.0 | 0.052 | 0.3 |
| 5 | 0.786 | 6.6 | 0.024 | 0.2 |
| 10 | 0.545 | 7.5 | 0.019 | 0.3 |
| 20 | 0.000 | — | 0.000 | — |
| citrulline (g/L) | | | | |
| 0 | 0.855 | 6.3 | 0.028 | 0.4 |
| 25 | 0.759 | 6.7 | 0.030 | 0.2 |
| 30 | 0.702 | 7.0 | 0.023 | 0.3 |
| 40 | 0.677 | 7.5 | 0.084 | 0.6 |
| 45 | 0.508 | 8.6 | 0.149 | 0.1 |
| 50 | 0.538 | 9.2 | 0.082 | 0.2 |
| 60 | 0.437 | 10.4 | 0.099 | 0.1 |
| 75 | 0.355 | 12.4 | 0.083 | 0.2 |
| ornithine (g/L) | | | | |
| 0 | 0.783 | 7.1 | 0.042 | 0.3 |
| 5 | 0.439 | 8.2 | 0.028 | 0.4 |
| 10 | 0.260 | 17.1 | 0.005 | 0.8 |
| 20 | 0.214 | 44.2 | 0.014 | 2.8 |
| 25 | 0.150 | 53.2 | 0.015 | 4.7 |
| 30 | 0.076 | 82.6 | 0.009 | 1.5 |
| 40 | 0.000 | — | 0.000 | — |

TABLE 37
Growth rates and lag times of putrescine-evolved isolates
in inhibitory concentrations (as shown) of ethylenediamine,
1,3-diaminopropane, cadaverine, and spermidine, as tested
in the Growth Profiler testing format.
A:

| | 18 g/L ethylenediamine | | | | 35 g/L 1,3-diaminopropane | | | |
|---|---|---|---|---|---|---|---|---|
| | mean (2) | | std. error (2) | | mean (2) | | std. error (2) | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.221 | 13.2 | 0.024 | 0.2 | 0.000 | — | 0.000 | — |
| PUTR2-4 | 0.191 | 13.8 | 0.021 | 1.5 | 0.178 | 54.8 | 0.064 | 2.4 |
| PUTR2-6 | 0.241 | 13.4 | 0.018 | 0.6 | 0.172 | 42.0 | 0.053 | 14.3 |
| PUTR3-1 | 0.431 | 8.6 | 0.019 | 0.6 | 0.223 | 26.6 | 0.003 | 0.8 |
| PUTR3-9 | 0.248 | 10.5 | 0.020 | 0.5 | 0.117 | 34.6 | 0.014 | 2.8 |
| PUTR3-10 | 0.403 | 9.1 | 0.009 | 0.4 | 0.232 | 27.4 | 0.008 | 3.6 |
| PUTR4-3 | 0.413 | 9.8 | 0.025 | 0.5 | 0.229 | 19.1 | 0.007 | 0.7 |
| PUTR4-7 | 0.367 | 12.0 | 0.008 | 0.2 | 0.225 | 23.8 | 0.006 | 3.3 |
| PUTR4-8 | 0.349 | 11.4 | 0.004 | 0.2 | 0.229 | 19.1 | 0.008 | 1.5 |
| PUTR5-1 | 0.405 | 9.8 | 0.032 | 0.6 | 0.094 | 32.2 | 0.002 | 5.3 |
| PUTR5-6 | 0.395 | 9.8 | 0.046 | 0.3 | 0.124 | 34.5 | 0.002 | 1.6 |
| PUTR5-8 | 0.408 | 9.4 | 0.009 | 0.2 | 0.156 | 31.4 | 0.024 | 1.3 |
| PUTR6-2 | 0.375 | 12.3 | 0.041 | 0.7 | 0.199 | 22.6 | 0.010 | 3.1 |
| PUTR6-7 | 0.486 | 9.1 | 0.015 | 1.4 | 0.251 | 23.7 | 0.014 | 2.4 |
| PUTR6-10 | 0.462 | 9.4 | 0.020 | 0.5 | 0.222 | 27.2 | 0.009 | 1.9 |
| PUTR7-1 | 0.356 | 9.6 | 0.007 | 0.1 | 0.140 | 19.3 | 0.011 | 1.3 |
| PUTR7-7 | 0.414 | 10.4 | 0.024 | 0.3 | 0.221 | 20.5 | 0.007 | 0.7 |
| PUTR7-9 | 0.380 | 10.1 | 0.042 | 0.7 | 0.226 | 20.2 | 0.003 | 0.3 |
| PUTR8-3 | 0.449 | 8.3 | 0.005 | 0.5 | 0.181 | 23.0 | 0.056 | 3.5 |
| PUTR8-6 | 0.369 | 8.3 | 0.029 | 1.1 | 0.138 | 23.8 | 0.008 | 0.6 |
| PUTR8-10 | 0.327 | 9.0 | 0.033 | 0.5 | 0.112 | 22.9 | 0.035 | 1.6 |

B:

| | 35 g/L cadaverine | | | | 40 g/L spermidine | | | |
|---|---|---|---|---|---|---|---|---|
| | mean (2) | | std. error (2) | | mean (2) | | std. error (2) | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.189 | 17.1 | 0.010 | 0.7 | 0.229 | 16.5 | 0.038 | 0.8 |
| PUTR2-4 | 0.345 | 13.7 | 0.043 | 0.7 | 0.214 | 16.0 | 0.023 | 0.6 |
| PUTR2-6 | 0.346 | 12.1 | 0.017 | 2.5 | 0.209 | 14.8 | 0.004 | 2.5 |
| PUTR3-1 | 0.477 | 10.0 | 0.024 | 0.1 | 0.369 | 11.3 | 0.011 | 0.0 |
| PUTR3-9 | 0.343 | 11.9 | 0.004 | 0.2 | 0.232 | 15.2 | 0.023 | 0.6 |
| PUTR3-10 | 0.469 | 10.2 | 0.011 | 0.4 | 0.385 | 11.1 | 0.007 | 0.5 |
| PUTR4-3 | 0.434 | 9.4 | 0.003 | 0.1 | 0.357 | 10.6 | 0.011 | 0.3 |
| PUTR4-7 | 0.448 | 10.2 | 0.024 | 0.5 | 0.363 | 11.7 | 0.010 | 0.5 |
| PUTR4-8 | 0.432 | 9.9 | 0.010 | 0.4 | 0.344 | 11.2 | 0.020 | 0.3 |
| PUTR5-1 | 0.329 | 12.7 | 0.006 | 0.3 | 0.215 | 14.0 | 0.042 | 0.6 |
| PUTR5-6 | 0.367 | 10.3 | 0.037 | 0.4 | 0.350 | 11.2 | 0.012 | 0.0 |
| PUTR5-8 | 0.355 | 10.3 | 0.018 | 0.1 | 0.338 | 11.2 | 0.012 | 0.2 |
| PUTR6-2 | 0.391 | 10.6 | 0.021 | 0.3 | 0.336 | 12.6 | 0.015 | 0.3 |
| PUTR6-7 | 0.449 | 10.0 | 0.013 | 0.6 | 0.350 | 11.2 | 0.009 | 0.6 |
| PUTR6-10 | 0.408 | 9.5 | 0.047 | 0.3 | 0.365 | 11.5 | 0.021 | 0.7 |
| PUTR7-1 | 0.352 | 10.5 | 0.077 | 0.3 | 0.302 | 11.3 | 0.015 | 0.2 |
| PUTR7-7 | 0.342 | 10.4 | 0.015 | 0.2 | 0.336 | 11.6 | 0.004 | 0.1 |
| PUTR7-9 | 0.376 | 9.9 | 0.024 | 0.1 | 0.327 | 11.8 | 0.003 | 0.4 |
| PUTR8-3 | 0.467 | 8.7 | 0.043 | 0.0 | 0.319 | 10.9 | 0.026 | 0.7 |
| PUTR8-6 | 0.437 | 8.8 | 0.074 | 0.6 | 0.308 | 10.5 | 0.007 | 0.7 |
| PUTR8-10 | 0.488 | 8.6 | 0.025 | 0.2 | 0.300 | 10.3 | 0.012 | 0.1 |

TABLE 38
Growth rates and lag times of HMDA-evolved isolates
in inhibitory concentrations (as shown) of ethylenediamine,
1,3-diaminopropane, cadaverine, and spermidine, as tested
in the Growth Profiler testing format.

| | 18 g/L ethylenediamine | | | | 35 g/L 1,3-diaminopropane | | | |
|---|---|---|---|---|---|---|---|---|
| | mean (2) | | std. error (2) | | mean (2) | | std. error (2) | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.162 | 15.8 | 0.019 | 0.4 | 0.000 | 0.0 | 0.000 | 0.0 |
| HMDA1-10 | 0.339 | 10.1 | 0.021 | 0.1 | 0.210 | 17.3 | 0.019 | 0.7 |
| HMDA2-1 | 0.224 | 17.7 | 0.057 | 3.0 | 0.076 | 26.2 | 0.008 | 0.9 |
| HMDA2-8 | 0.165 | 13.7 | 0.020 | 1.3 | 0.073 | 25.3 | 0.004 | 0.8 |
| HMDA3-4 | 0.308 | 12.3 | 0.041 | 0.3 | 0.106 | 41.3 | 0.046 | 18.5 |
| HMDA3-5 | 0.319 | 12.4 | 0.012 | 0.5 | 0.000 | — | 0.000 | — |
| HMDA3-6 | 0.276 | 13.3 | 0.054 | 0.6 | 0.083 | 40.0 | 0.081 | 2.4 |
| HMDA4-2 | 0.286 | 18.5 | 0.013 | 0.3 | 0.118 | 20.4 | 0.004 | 0.9 |
| HMDA4-6 | 0.298 | 18.8 | 0.008 | 0.7 | 0.167 | 18.8 | 0.014 | 0.7 |
| HMDA4-9 | 0.212 | 18.1 | 0.019 | 4.6 | 0.139 | 18.6 | 0.023 | 0.6 |
| HMDA5-4 | 0.406 | 12.5 | 0.020 | 0.6 | 0.000 | — | 0.000 | — |
| HMDA5-5 | 0.439 | 12.9 | 0.027 | 0.5 | 0.000 | — | 0.000 | — |
| HMDA5-10 | 0.439 | 16.9 | 0.020 | 0.7 | 0.100 | 29.2 | 0.030 | 2.0 |
| HMDA6-3 | 0.312 | 11.9 | 0.006 | 0.9 | 0.195 | 34.8 | 0.051 | 1.5 |
| HMDA6-7 | 0.350 | 11.1 | 0.008 | 0.5 | 0.174 | 33.6 | 0.020 | 2.3 |
| HMDA7-1 | 0.000 | — | 0.000 | — | 0.054 | 29.5 | 0.020 | 0.7 |
| HMDA7-7 | 0.000 | — | 0.000 | — | 0.055 | 31.0 | 0.005 | 2.5 |
| HMDA7-10 | 0.000 | — | 0.000 | — | 0.062 | 30.8 | 0.013 | 0.9 |
| HMDA8-5 | 0.203 | 24.9 | 0.104 | 7.8 | 0.134 | 23.3 | 0.023 | 0.4 |
| HMDA8-9 | 0.104 | 16.7 | 0.090 | 14.6 | 0.130 | 23.1 | 0.023 | 1.3 |
| HMDA8-10 | 0.158 | 29.4 | 0.062 | 5.2 | 0.106 | 19.4 | 0.037 | 2.0 |

| | 35 g/L cadaverine | | | | 40 g/L spermidine | | | |
|---|---|---|---|---|---|---|---|---|
| | mean (2) | | std. error (2) | | mean (2) | | std. error (2) | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.169 | 17.1 | 0.032 | 0.7 | 0.232 | 17.4 | 0.013 | 0.5 |
| HMDA1-10 | 0.365 | 8.8 | 0.007 | 0.2 | 0.243 | 49.5 | 0.045 | 4.2 |
| HMDA2-1 | 0.406 | 9.8 | 0.022 | 0.2 | 0.082 | 23.6 | 0.009 | 0.6 |
| HMDA2-8 | 0.418 | 9.7 | 0.011 | 0.4 | 0.073 | 23.4 | 0.010 | 1.4 |
| HMDA3-4 | 0.521 | 9.2 | 0.004 | 0.6 | 0.359 | 12.4 | 0.020 | 1.1 |
| HMDA3-5 | 0.532 | 9.6 | 0.004 | 0.4 | 0.393 | 16.0 | 0.004 | 1.0 |
| HMDA3-6 | 0.524 | 9.3 | 0.019 | 0.3 | 0.363 | 11.6 | 0.045 | 1.0 |
| HMDA4-2 | 0.469 | 8.5 | 0.025 | 0.3 | 0.275 | 10.8 | 0.007 | 0.4 |
| HMDA4-6 | 0.498 | 8.3 | 0.024 | 0.0 | 0.261 | 12.5 | 0.017 | 0.8 |
| HMDA4-9 | 0.475 | 8.2 | 0.011 | 0.1 | 0.241 | 12.0 | 0.008 | 0.0 |
| HMDA5-4 | 0.526 | 10.2 | 0.011 | 0.3 | 0.372 | 16.2 | 0.037 | 1.5 |
| HMDA5-5 | 0.533 | 9.2 | 0.008 | 0.7 | 0.398 | 12.8 | 0.037 | 1.5 |
| HMDA5-10 | 0.404 | 10.6 | 0.008 | 0.1 | 0.336 | 12.2 | 0.009 | 0.1 |
| HMDA6-3 | 0.429 | 9.4 | 0.021 | 0.1 | 0.214 | 12.7 | 0.011 | 0.8 |
| HMDA6-7 | 0.461 | 9.5 | 0.010 | 0.2 | 0.247 | 12.8 | 0.011 | 0.2 |
| HMDA7-1 | 0.433 | 8.7 | 0.007 | 0.2 | 0.241 | 12.0 | 0.006 | 0.3 |
| HMDA7-7 | 0.439 | 9.2 | 0.000 | 0.4 | 0.227 | 11.9 | 0.023 | 0.1 |
| HMDA7-10 | 0.460 | 8.9 | 0.011 | 0.4 | 0.241 | 12.6 | 0.015 | 0.7 |
| HMDA8-5 | 0.496 | 9.3 | 0.002 | 0.1 | 0.291 | 14.9 | 0.029 | 0.7 |
| HMDA8-9 | 0.498 | 9.3 | 0.020 | 0.2 | 0.284 | 15.2 | 0.013 | 0.1 |
| HMDA8-10 | 0.497 | 8.7 | 0.015 | 0.6 | 0.248 | 15.0 | 0.024 | 0.4 |

TABLE 39
Growth rates and lag times of putrescine-evolved isolates
in inhibitory concentrations (as shown) of citrulline and
ornithine, as tested in the Growth Profiler testing format.

| | 80 g/L citrulline | | | | 10 g/L ornithine | | | |
|---|---|---|---|---|---|---|---|---|
| | mean (2) | | std. error (2) | | mean (2) | | std. error (2) | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.347 | 10.3 | 0.029 | 0.2 | 0.331 | 12.8 | 0.003 | 0.3 |
| PUTR2-4 | 0.493 | 11.4 | 0.024 | 0.3 | 0.697 | 6.9 | 0.116 | 0.5 |
| PUTR2-6 | 0.508 | 11.0 | 0.016 | 0.5 | 0.775 | 7.1 | 0.116 | 0.1 |
| PUTR3-1 | 0.601 | 8.0 | 0.021 | 0.5 | 0.612 | 4.7 | 0.101 | 0.2 |
| PUTR3-9 | 0.414 | 8.7 | 0.077 | 0.3 | 0.521 | 5.4 | 0.041 | 0.1 |
| PUTR3-10 | 0.599 | 8.2 | 0.002 | 0.7 | 0.638 | 4.8 | 0.060 | 0.2 |
| PUTR4-3 | 0.571 | 8.2 | 0.012 | 0.4 | 0.928 | 5.4 | 0.110 | 0.1 |
| PUTR4-7 | 0.569 | 9.4 | 0.018 | 0.3 | 0.837 | 5.8 | 0.020 | 0.1 |
| PUTR4-8 | 0.536 | 9.2 | 0.034 | 0.3 | 0.794 | 5.6 | 0.037 | 0.2 |
| PUTR5-1 | 0.385 | 9.9 | 0.007 | 0.1 | 0.405 | 7.6 | 0.078 | 0.7 |
| PUTR5-6 | 0.463 | 9.5 | 0.009 | 0.1 | 0.855 | 5.6 | 0.018 | 0.1 |
| PUTR5-8 | 0.432 | 8.6 | 0.088 | 0.3 | 0.885 | 5.5 | 0.008 | 0.1 |
| PUTR6-2 | 0.433 | 11.0 | 0.007 | 1.2 | 0.391 | 17.1 | 0.050 | 0.7 |
| PUTR6-7 | 0.575 | 8.7 | 0.014 | 0.8 | 0.938 | 4.8 | 0.040 | 0.3 |
| PUTR6-10 | 0.534 | 8.5 | 0.003 | 0.0 | 0.864 | 5.2 | 0.018 | 0.2 |
| PUTR7-1 | 0.202 | 13.1 | 0.058 | 1.1 | 0.659 | 6.1 | 0.029 | 0.0 |
| PUTR7-7 | 0.471 | 11.1 | 0.004 | 0.1 | 0.865 | 5.8 | 0.028 | 0.1 |
| PUTR7-9 | 0.460 | 11.4 | 0.030 | 0.5 | 0.782 | 5.5 | 0.052 | 0.1 |
| PUTR8-3 | 0.542 | 7.9 | 0.031 | 0.4 | 0.724 | 4.9 | 0.099 | 0.2 |
| PUTR8-6 | 0.574 | 7.5 | 0.016 | 0.2 | 0.720 | 4.7 | 0.025 | 0.2 |
| PUTR8-10 | 0.575 | 8.0 | 0.008 | 0.6 | 0.720 | 4.9 | 0.037 | 0.1 |

TABLE 40
Growth rates and lag times of HMDA-evolved isolates in inhibitory
concentrations (as shown) of citrulline and ornithine, as
tested in the Growth Profiler testing format.

| | 80 g/L citrulline | | | | 10 g/L ornithine | | | |
|---|---|---|---|---|---|---|---|---|
| | mean (2) | | std. error (2) | | mean (2) | | std. error (2) | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.296 | 12.2 | 0.007 | 0.1 | 0.375 | 13.3 | 0.020 | 0.4 |
| HMDA1-10 | 0.601 | 9.1 | 0.019 | 0.1 | 0.764 | 6.3 | 0.047 | 0.1 |
| HMDA2-1 | 0.445 | 11.6 | 0.103 | 1.4 | 0.725 | 6.0 | 0.018 | 0.3 |
| HMDA2-8 | 0.516 | 9.8 | 0.008 | 0.2 | 0.760 | 5.6 | 0.013 | 0.1 |
| HMDA3-4 | 0.637 | 11.3 | 0.013 | 0.7 | 0.911 | 7.8 | 0.031 | 0.3 |
| HMDA3-5 | 0.547 | 10.1 | 0.167 | 0.2 | 0.835 | 5.9 | 0.006 | 0.1 |
| HMDA3-6 | 0.655 | 10.7 | 0.030 | 0.2 | 0.901 | 8.0 | 0.033 | 0.1 |
| HMDA4-2 | 0.561 | 16.2 | 0.021 | 0.6 | 0.814 | 10.3 | 0.058 | 0.3 |
| HMDA4-6 | 0.525 | 16.7 | 0.116 | 0.9 | 0.801 | 12.5 | 0.070 | 0.6 |
| HMDA4-9 | 0.583 | 13.0 | 0.005 | 2.8 | 0.761 | 8.5 | 0.031 | 0.9 |
| HMDA5-4 | 0.627 | 9.5 | 0.089 | 0.4 | 0.750 | 5.6 | 0.013 | 0.1 |
| HMDA5-5 | 0.654 | 9.6 | 0.015 | 0.2 | 0.783 | 4.9 | 0.035 | 0.1 |
| HMDA5-10 | 0.513 | 11.7 | 0.021 | 0.2 | 0.818 | 6.5 | 0.094 | 0.4 |
| HMDA6-3 | 0.402 | 10.8 | 0.255 | 3.1 | 0.852 | 5.2 | 0.081 | 0.3 |
| HMDA6-7 | 0.565 | 8.2 | 0.006 | 0.4 | 0.810 | 5.3 | 0.030 | 0.1 |
| HMDA7-1 | 0.518 | 27.1 | 0.010 | 1.0 | 0.810 | 7.1 | 0.012 | 0.1 |
| HMDA7-7 | 0.462 | 27.7 | 0.103 | 1.0 | 0.810 | 7.3 | 0.009 | 0.1 |
| HMDA7-10 | 0.507 | 27.7 | 0.007 | 1.5 | 0.807 | 7.2 | 0.014 | 0.2 |
| HMDA8-5 | 0.487 | 15.8 | 0.012 | 0.7 | 0.715 | 6.3 | 0.034 | 0.1 |
| HMDA8-9 | 0.463 | 16.8 | 0.088 | 1.5 | 0.746 | 6.4 | 0.106 | 0.3 |
| HMDA8-10 | 0.497 | 15.6 | 0.002 | 0.5 | 0.887 | 6.6 | 0.078 | 0.1 |

l) Biological Production of Polyamines

E. coli has been metabolically engineered to produce up to 24.2 g/L putrescine from glucose (Qian et al., Biotechnol. Bioeng. 104:651-662, 2009). A schematic of the modifications employed in the overproducing E. coli K-12 W3110 strain is shown in FIG. 1 of Qian et al. (2009), which figure is hereby specifically incorporated by reference. Briefly, the native E. coli pathway leading to L-ornithine was employed, with the ArgB, ArgC, ArgD, and ArgE overexpressed by replacing the native promoter of the argBCDE operon with an inducible Ptrc promoter. The promoter for the speF-potE promoter was also replaced with an inducible Ptrc promoter, with PotE being a putrescine export protein. The native promoter for speC, encoding an ornithine decarboxylase responsible for converting L-ornithine to putrescine, was also replaced with an inducible Ptrc promoter to increase its expression, and this gene was additionally overexpressed off a plasmid (p15SpeC). The argI, speE, speG, and puuPA operons were deleted from the genome to prevent putrescine conversion to other products, conversion of L-ornithine to L-arginine, and putrescine re-import via the PuuP importer. The best producing strain in fed-batch fermentations (XQ52/p15SpeC) also contained a deletion of rpoS, which encodes the stationary phase sigma factor.

XQ52 and p15SpeC were generously donated by S. Y. Lee (Qian et al., 2009) and were used to conduct two types of screens for putrescine production. In the first screen, evolved isolates were transformed with p15SpeC to allow a low level of putrescine overproduction in an otherwise unmodified background strain. They were compared for putrescine overproduction with K-12 MG1655 harboring p15SpeC, and XQ52 harboring p15SpeC as a positive control in a batch screen as described in the Methods (Table 41. After 24 hours, a number of evolved isolates exhibited higher putrescine titers than the K-12 MG1655 control, most notably PUTR3-1, PUTR5-8, PUTR6-7, PUTR7-7, and PUTR7-9. After 48 hours, strains with the highest production were PUTR5-6, PUTR5-8, PUTR7-1, PUTR7-7, and PUTR7-9. This includes all isolates that contained the E575A mutation in RpoD (encoding the housekeeping sigma factor, sigma 70), indicating its causation in improved endogenous production of putrescine. PUTR5-6 and PUTR5-8 contained an additional mutation in RpoC (V401G), while PUTR7-7 and PUTR7-9 contained additional mutations in MurA (Y393S) and RpoB (R637L). Without being limited to theory, the MurA mutation was believed to be responsible for the reduced FSC values observed in Table 34. PUTR7-1, by contrast, harbored mutations in RpsA (D310Y), NusA (M204R), MreB (H93N), and SpoT (R467H). Without being limited to theory, the MreB-H93N mutation was also believed to be responsible for the reduced FSC values observed in Table 34.

TABLE 41
Batch production screen for putrescine overproduction in XQ52,
K-12 MG1655, and evolved isolates harboring plasmid p15SpeC,
with titers measured after 24 and 48 hours cultivation.

putrescine titer (g/L)

| | 24 h | 48 h |
|---|---|---|
| XQ52/p15SpeC | 0.35 | 1.01 |
| MG1655/p15SpeC | 0.15 | 0.22 |
| PUTR2-4/p15SpeC | 0.18 | 0.31 |
| PUTR2-6/p15SpeC | 0.20 | 0.27 |
| PUTR3-1/p15SpeC | 0.30 | 0.32 |
| PUTR3-9/p15SpeC | 0.23 | 0.28 |
| PUTR3-10/p15SpeC | 0.22 | 0.34 |
| PUTR4-3/p15SpeC | 0.12 | 0.15 |
| PUTR4-7/p15SpeC | 0.18 | 0.19 |
| PUTR4-8/p15SpeC | 0.21 | 0.27 |
| PUTR5-1/p15SpeC | 0.13 | 0.18 |
| PUTR5-6/p15SpeC | 0.16 | 0.43 |
| PUTR5-8/p15SpeC | 0.29 | 0.41 |
| PUTR6-2/p15SpeC | 0.15 | 0.13 |
| PUTR6-7/p15SpeC | 0.25 | 0.23 |
| PUTR6-10/p15SpeC | 0.16 | 0.32 |
| PUTR7-1/p15SpeC | 0.07 | 0.44 |
| PUTR7-7/p15SpeC | 0.39 | 0.48 |
| PUTR7-9/p15SpeC | 0.35 | 0.39 |
| PUTR8-3/p15SpeC | 0.16 | 0.19 |
| PUTR8-6/p15SpeC | 0.19 | 0.24 |
| PUTR8-10/p15SpeC | 0.14 | 0.22 |

The best producing strains from Table 36 were grown in semi-batch cultivation with a glucose/ammonium sulfate/magnesium sulfate feed solution as described in Methods. In this condition, higher cell densities and putrescine titers were achieved (Table 42). The PUTR7-9 background exhibited the highest level of production (4.46 g/L compared to 3.73 g/L in K-12 MG1655), as well as the highest specific production normalized to cell density. The PUTR3-10 background also exhibited a slightly higher titer than K-12 MG1655.

TABLE 42
Semi-batch production screen (with glucose/ammonium
sulfate/magnesium sulfate feeding) for putrescine overproduction
in K-12 MG1655 and selected evolved isolates harboring
plasmid p15SpeC. Titers and specific production were
measured after 48 hours cultivation.

putrescine production at 48 h

| strain | g/L | g/L/OD₆₀₀ |
|---|---|---|
| K-12 MG1655/p15SpeC | 3.73 | 0.131 |
| PUTR3-1/p15SpeC | 3.67 | 0.118 |
| PUTR3-10/p15SpeC | 3.86 | 0.122 |
| PUTR5-6/p15SpeC | 2.32 | 0.082 |
| PUTR5-8/p15SpeC | 3.53 | 0.110 |
| PUTR7-1/p15SpeC | 2.85 | 0.129 |
| PUTR7-7/p15SpeC | 3.52 | 0.110 |
| PUTR7-9/p15SpeC | 4.46 | 0.141 |

In a second type of screen, the highly modified background strain XQ52 was modified by MAGE to introduce the most beneficial point mutations found for improving tolerance.

Plasmid p15SpeC was reintroduced into each background strain to generate the final production strain. In batch screening (Table 43, the ygaC and sspA mutant backgrounds were found to have slightly higher titers than XQ52 after 24 hours. After 48 hours, the mreB, argG, rpsG, and rpsG argG mutants exhibited the highest putrescine titers, with all mutants exhibiting higher titers than XQ52. In semi-batch cultivation with glucose/ammonium sulfate/magnesium sulfate feeding (Table 44, higher cell densities and putrescine titers were achieved, although they were again much lower than those published by Qian et al. (2009) and below exogenously toxic concentrations of putrescine. After 24 hours, the argG mutant exhibited a moderately increased titer compared with the XQ52 background. However after 48 hours, XQ52 exhibited the highest production.

TABLE 43
Batch production screen for putrescine overproduction
in evolved isolates PUTR4-3, PUTR8-10, XQ52, K-12
MG1655, and XQ52 harboring MAGE-generated tolerance
mutations, containing plasmid p15SpeC. Titers were
measured after 24 and 48 hours cultivation.

| | putrescine titer (g/L) | | | |
|---|---|---|---|---|
| | mean | | standard error | |
| strain | 24 h | 48 h | 24 h | 48 h |
| PUTR8-10/p15SpeC | 0.49 | 0.43 | 0.03 | 0.10 |
| PUTR4-3/p15SpeC | 0.43 | 0.27 | 0.01 | 0.02 |
| XQ52/p15SpeC | 0.93 | 1.51 | 0.20 | 0.10 |
| XQ52 argG*/p15SpeC | 0.93 | 1.85 | 0.11 | 0.10 |
| XQ52 mreB*/p15SpeC | 0.66 | 1.87 | 0.08 | 0.02 |
| XQ52 ygaC*/p15SpeC | 0.97 | 1.80 | 0.05 | 0.05 |
| XQ52 rpsG*/p15SpeC | 0.67 | 1.84 | 0.05 | 0.01 |
| XQ52 sspA*/p15SpeC | 1.07 | 1.63 | 0.01 | 0.01 |
| XQ52 rpsG* argG*/p15SpeC | 0.65 | 1.84 | 0.05 | 0.06 |
| XQ52 rpsG* mreB*/p15SpeC | 0.58 | 1.72 | 0.03 | 0.06 |

TABLE 44
Semi-batch production screen (with glucose/ammonium
sulfate/magnesium sulfate feeding) for putrescine
overproduction in XQ52 and XQ52 with MAGE-generated
tolerance mutations, harboring plasmid p15SpeC.
Titers and specific production were measured after
48 hours cultivation.

| | putrescine | | putrescine | |
|---|---|---|---|---|
| | production | | production | |
| | at 24 h | | at 48 h | |
| strain | g/L | g/L/OD₆₀₀ | g/L | g/L/OD₆₀₀ |
| XQ52/p15SpeC | 2.21 | 0.157 | 7.66 | 0.267 |
| XQ52 argG*/p15SpeC | 2.67 | 0.232 | 7.50 | 0.268 |
| XQ52 mreB*/p15SpeC | 1.83 | 0.208 | 4.74 | 0.251 |
| XQ52 ygaC*/p15SpeC | 1.91 | 0.203 | 6.31 | 0.237 |
| XQ52 rpsG*/p15SpeC | 1.81 | 0.226 | 6.07 | 0.244 |
| XQ52 sspA*/p15SpeC | 1.90 | 0.183 | 5.53 | 0.241 |
| XQ52 rpsG* argG*/p15SpeC | 1.42 | 0.170 | 5.57 | 0.230 |
| XQ52 rpsG* mreB*/p15SpeC | 1.05 | 0.158 | 5.72 | 0.242 |

Production of putrescine from the Gram-positive bacteria Corynebacterium glutamicum, with a maximum reported titer of 88 g/L, has been reported (Kind et al., 2014; Jensen et al., 2015; Nguyen et al., 2015; Schneider et al., 2012; Meiswinkel et al., 2013). There has been intensive interest in employing this microorganism for the production of putrescine and other polyamines due to their derivation from L-glutamate and L-lysine, two amino acids that are almost exclusively produced at high titer in this organism.

Cadaverine is a 5-carbon diamine intermediate in chain length between putrescine and HMDA. It was not employed in our evolution experiments due to its high cost, however it is highly likely that many of the putrescine and HMDA evolved strains are cross-tolerant to cadaverine, and this will be tested in the future. Cadaverine is natively produced in E. coli and derives directly from L-lysine via CadA (lysine decarboxylase). It has been reported that up to 9.6 g/L was produced in fed-batched fermentations using engineered E. coli K-12 W3110 (Qian et al., Biotechnol Bioeng. 108:93-103, 2011). The modifications to this strain were deletion of speE, speG, puuA, and ygjG which convert cadaverine to other products, and puuP, which re-imports cadaverine from the extracellular medium, as shown in FIG. 1 of Qian et al. (2011), which is specifically incorporated by reference. Various genes in the pathway leading to L-lysine were overexpressed however only the replacement of the native dapA promoter with the Ptrc promoter was necessary to achieve the highest reported titer. CadA was additionally overexpressed off a plasmid (p15CadA). Attempts were made to reduce acetate production by deletion of iclR, however this modification did not improve cadaverine production.

Cadaverine has more successfully been produced in Corynebacterium glutamicum, with a maximum reported titer of 88 g/L by fed-batch fermentation (Kind et al., Metab. Eng. 25:113-123, 2014). This was obtained in a pre-existing highly engineered lysine-overproducing strain that possessed various genome modifications resulting in deregulation and redirection of flux into lysine production. Additional modifications to this strain included the genome integration of a codon-optimized E. coli ldcC (an alternative lysine decarboxylase in E. coli to CadA), deletion of a C. glutamicum N-acetyltransferase that converts cadaverine to N-acetylcadaverine, deletion of lysE encoding the lysine exporter, and overexpression of a C. glutamicum permease responsible for exporting cadaverine.

A summary of known biological pathways for producing polyamines and other and monomers for the production of polymers is shown in FIG. 2 of Chung et al. (2015), which is hereby incorporated by reference in its entirety. In addition, Chae et al. (2015) and Qian et al. (2009, 2011), also incorporated by reference in their entireties, have reported metabolic engineering of E. coli for the production of 1,3-diaminopropane, putrescine and cadaverine.

Finally, as to biological production of hexamethylenediamine, FIGS. 10, 11, 13, 20, 21, 22, 24, 25, and 26 of US patent application publication No. 2012/0282661 A1 (Genomatica Inc.), which are hereby incorporated by reference in their entireties, describe biological pathways leading to HMDA from different precursors. This publication describes a recombinant cell that can produce 6-aminocaproic acid, and a recombinant cell that comprises an enzyme with 2-oxoheptane-1,7-dioate aminotransferase activity, or 2-oxoheptane-1,7-dioate decarboxylase activity, and 6-aminocaproic acid is a precursor for HMDA via a few enzymatic steps. Additional examples are shown for production of HMDA via succinyl-CoA and acetyl-CoA, 4-aminobutyryl-CoA and acetyl-CoA, glutamate, glutaryl-CoA, pyruvate and 4-aminobutanal, and 2-amino-7-oxosubarate. Additional pathways describing routes to some of these precursors from natively occurring precursors are also described.

### LIST OF REFERENCES

Adkins J, Jordan J, Nielsen D R. Engineering Escherichia coli for renewable production of the 5-carbon polyamide building-blocks 5-aminovalerate and glutarate. Biotechnol. Bioeng. 110:1726-1734 (2013).
Al Zaid Siddiquee K, Arauzo-Bravo M J, Shimizu K. Metabolic flux analysis of pykF gene knockout Escherichia coli based on 13C-labeling experiments together with measurements of enzyme activities and intracellular metabolite concentrations. Appl. Microbiol. Biotechnol. 63:407-417 (2004).
Atsumi S, Wu T Y, Machado I M P, Huang W C, Chen P Y, Pellegrini M, Liao J C. Evolution, genomic analysis, and reconstruction of isobutanol tolerance in Escherichia coli. Mol. Sys. Bio. 6:449 (2010).
Baba T et al., Construction of Escherichia coli K-12 in-frame, single-gene knockout mutants: the Keio collection. Mol. Syst. Biol. 2:2006.0008 (2006).
Chae et al., Metabolic engineering of Escherichia coli for the production of 1,3-diaminopropane, a three carbon diamine. Sci Rep. 2015; 5: 13040.
Chung et al. Bio-based production of monomers and polymers by metabolically engineered microorganisms. Curr. Opin. Biotechnol., 36:73-84, 2015.
Datsenko K A, Wanner B L. One-step inactivation of chromosomal genes in Escherichia coli K-12 using PCR products. Proc. Natl. Acad. Sci. USA 97:6640-6645 (2000).
Doukyu N, Ishikawa K, Watanabe R, Ogino H. Improvement in organic solvent tolerance by double disruption of proV and marR genes in Escherichia coli. J. Appl. Microbiol. 112:464-474 (2012).
Dragosits M, Mozhayskiy V, Quinones-Soto S, Park J, Tagkopoulos I. Evolutionary potential, cross-stress behavior and the genetic basis of acquired stress resistance in Escherichia coli. Mol. Syst. Biol. 9:643 (2013).
Dragosits M, Mattanovich D. Adaptive laboratory evolution—principles and applications for biotechnology, Microbial Gell Factories 12:64 (2013).
Eberhardt D, Jensen J V K, Wendisch V F. L-citrulline production by metabolically engineered Corynebacterium glutamicum from glucose and alternative carbon sources. AMB Express 4:85 (2014).
Haft R J F, et al. Correcting direct effects of ethanol on translation and transcription machinery confers ethanol tolerance in bacteria. Proc. Natl. Acad. Sci. USA 111:E2576-E2585 (2014).
Hwang J H, Hwang G H, Cho J Y. Effect of increased glutamate availability on L-ornithine production in Corynebacterium glutamicum. J. Microbiol. Biotechnol. 18:704-710 (2008).
Jensen J V, Eberhardt D, Wendisch V F. Modular pathway engineering of Corynebacterium glutamicum for production of the glutamate-derived compounds ornithine, proline, putrescine, citrulline, and arginine. J. Biotechnol. 214:85-94 (2015).
Jensen S I, Lennen R M, Herrgård M J, Nielsen A T. Seven deletions in seven days: Fast generation of Escherichia coli strains tolerant to acetate and osmotic stress. Sci. Rep., accepted.
Kind S, Neubauer S, Becker J, Yamamoto M, Volkert M, Abendroth Gv, Zelder O, Wittmann C. From zero to hero—production of bio-based nylon from renewable resources using engineered Corynebacterium glutamicum. Metab. Eng. 25:113-123 (2014).
LaCroix R A, et al. Use of adaptive laboratory evolution to discover key mutations enabling rapid growth of Escherichia coli K-12 MG1655 on glucose minimal medium. Appl. Environ. Microbiol. 81:17-30 (2015).
Lee J W, et al. Microbial production of building block chemicals and polymers. Curr. Opin. Biotechnol. 22:758-767 (2011).
Lennen R M, Herrgård M J. Combinatorial strategies for improving multiple-stress resistance in industrially relevant Escherichia coli strains. Appl. Environ. Microbiol. 80:6223-6242 (2014).
Meiswinkel T M, Rittmann D, Lindner S N, Wendisch V F. Crude glycerol-based production of amino acids and putrescine by Corynebacterium glutamicum. Bioresour. Technol. 145:254-258 (2013).
Minty J J, et al. Evolution combined with genomic study elucidates genetic bases of isobutanol tolerance in Escherichia coli. Microbial Cell Factories 10:18 (2011).
Nguyen A Q, Schneider J, Reddy G K, Wendisch V F. Fermentative production of the diamine putrescine: system metabolic engineering of Corynebacterium glutamicum. Metabolites 5:211-231 (2015).
Nguyen A Q, Schneider J, Wendisch V F. Elimination of polyamine N-acetylation and regulatory engineering improved putrescine production by Corynebacterium glutamicum. 3. Biotechnol. 201:75-85 (2015).

Nicolaou S A, et al. A comparative view of metabolite and substrate stress and tolerance in microbial bioprocessing: from biofuels and chemicals, to biocatalysis and bioremediation. Metab. Eng. 12:307-331 (2010).
Qian Z G, Xia X X, Lee S Y. Metabolic engineering of Escherichia coli for the production of putrescine: a four carbon diamine. Biotechnol. Bioeng. 104:651-662 (2009).
Qian Z G, et al., Biotechnol Bioeng. Metabolic engineering of Escherichia coli for the production of cadaverine: A five carbon diamine. 108:93-103, 2011.
Quandt E M, Deatherage D E, Ellington A D, Georgiou G, Barrick J E. Recursive genomewide recombination and sequencing reveals a key refinement step in the evolution of a metabolic innovation in Escherichia coli. Proc. Natl. Acad. Sci. USA 111:2217-2222 (2014).
Rath D, Jawali N. Loss of expression of cspC, a cold shock family gene, confers a gain of fitness in Escherichia coli K-12 strains. 3. Bacteriol. 188:6780-6785 (2006).
Sandberg T E, et al. Evolution of Escherichia coli to 42° C. and subsequent genetic engineering reveals adaptive mechanisms and novel mutations. Mol. Biol. Evol. 31:2647-2662 (2014).
Schneider J, Eberhardt D, Wendisch V F. Improving putrescine production by Corynebacterium glutamicum by fine-tuning ornithine transcarbamoylase activity using a plasmid addiction system. Appl. Microbiol. Biotechnol. 95:169-178 (2012).

Shen C R, Lan E I, Dekishima Y, Baez A, Cho K M, Liao J C. Driving forces enable high-titer anaerobic 1-butanol synthesis in Escherichia coli. Appl. Environ. Microbiol. 77:2905-2915 (2011).
Shenhar Y, Biran D, Zon E Z. Resistance to environmental stress requires the RNA chaperones CspC and CspE. Environ. Microbiol. Rep. 4:532-539 (2012).
Tenaillon O, Rodriguez-Verdugo A, Gaut R L, McDonald P, Bennett A F, Long A D, Gaut B S. The molecular diversity of adaptive convergence. Science 335:457-461 (2012).
Thomason L C, Constantino N, Court D L. E. coli genome manipulation by P1 transduction. Curr. Protoc. Molec. Biol. 1.17 (2007).
Thomason L, Court D L, Bubunenko M, Constantino N, Wilson H, Datta S, Oppenheim A. Recombineering: Genetic engineering in bacteria using homologous recombination. Curr. Protoc. Molec. Biol. 1.16 (2007).
Van Dien S. From the first drop to the first truckload: commercialization of microbial processes for renewable chemicals. Gurr. Opin. Biotechno. 24:1-8 (2013).
Wang H H, Isaacs F J, Carr P A, Sun Z Z, Xu G, Forest C R, Church G M. Programming cells by multiplex genome engineering and accelerated evolution. Nature 460:894-898 (2009).
Warnecke T, Gill R T. Organic acid toxicity, tolerance, and production in Escherichia coli biorefining applications. Microbial Gell Factories 4:25 2005.
Wendisch V F. Microbial production of amino acid-related compounds. Adv. Biochem. Eng. Biotechnol. 2016 Nov. 22 (Epub ahead of print)
Winkler 3D, et al. Evolved osmotolerant Escherichia coli mutants frequently exhibit defective Nacetylglucosamine catabolism and point mutations in the cell shape-regulating protein MreB. Appl. Environ. Microbiol. 80:3729-3740 (2014).
Woodruff L B, Boyle N R, Gill R T. Engineering improved ethanol production in Escherichia coli with a genome-wide approach. Metab. Eng. 17:1-11 (2013).
Wu X, Altman R, Eiteman M A, Altman E. Adaptation of Escherichia coli to elevated sodium concentrations increases cation tolerance and enables greater lactic acid production. Appl. Environ. Microbiol. 80:2880-2888 (2014).
Zhang K, Woodruff A P, Xiong M, Zhou 3, Dhande Y K. A synthetic metabolic pathway for production of the platform chemical isobutyric acid. GhemSusGhem 4:1068-1070 (2011).
Zingaro K A, et al. Dissecting the assays to assess microbial tolerance to toxic chemicals in bioprocessing. Trends Biotechnol. 31:643-653 (2013).
US 2012/0282661 A1 (Genomatica Inc.)
WO 2009/086075 A1 and US 2009/0162911 A1 (E I Du Pont De Nemours and Company)
US 2003/0068611 A1 (E I DuPont De Nemours and Company)
WO 2009/143455 A2 (Microbia Inc.)
WO 2011/154503 A1 (Evonik Degussa GmbH)
US 2014/0135526 A1 (OPX Biotechnologies Inc.)
WO 2014/029592 A1 (Evonik Degussa GmbH)
EP 2 032 711 A1 (TNO)
EP 2 580 315 A2 (Cobalt Technologies Inc.)

1. A bacterial cell comprising a recombinant biosynthetic pathway for producing an aliphatic polyamine and at least one genetic modification which reduces expression of an endogenous gene selected from the group consisting of proV, proW, proX, cspC, ptsP, wbbK, yobF, nagC, nagA, rph, ybeX and mpl, or a combination of any thereof.

2. The bacterial cell of claim 1, comprising a genetic modification which reduces expression of ybeX, proV, cspC, ptsP, wbbK, mpl or rph.

3. The bacterial cell of claim 2, comprising genetic modifications which reduce the expression of
a) proV and at least one of ptsP, cspC, mpl, and ybeX;
b) proV, ptsP, and at least one of mpl and ybeX;
c) proV, cspC, and at least one of mpl and ybeX;
d) ybeX and at least one of proV, ptsP, cspC, and mpl;
e) proV, ptsP, ybeX, and mpl; or
f) proV, cspC, ybeX, and mpl.

4. The bacterial cell of claim 1, wherein the genetic modification comprises a knock-down or knock-out of the endogenous gene.

5. The bacterial cell of claim 1, further comprising a mutation in at least one of YgaC, RpsG, MreB, NusA, SspA, MrdB, RpoD, RpoC, RpoB, MurA, RpsA, SpoT, argG, rph or the pyrE/rph intergenic region.

6. A bacterial cell comprising at least one mutation selected from YgaC-R43L, RpsG-L157*, MreB-A298V, MreB-N34K, MreB-E212A, MreB-I24M, MreB-H93N, NusA-L152R, NusA-M204R, SspA-F83C, SspA-V91F, MrdB-E254K, RpoD-E575A, RpoC-V401G, RpoC-V453I, RpoC-R1140C, RpoC-L120P, RpoB-R637L, RpoB-G181V, MurA-G141A, MurA-Y393S, RpsA-D310Y, RpsA-D310G, RpsA-D160V, RpsA-N313K, RpsA-N315K, RpsA-E427R, SpoT-R209H, SpoT-R467H, SpoT-R471H, SpoT-R488C, SpoT-G530C and argG-C324A.

7. The bacterial cell of claim 1, wherein the genetic modification provides for an increased growth rate, a reduced lag time, or both, of the cell in at least one of putrescine, hexamethylenediamine (HMDA), 1,3-diaminopropane, spermidine, agmatine, cadaverine, ethylenediamine, citrulline, and ornithine as compared to the bacterial cell without the genetic modification.

8. The bacterial cell of claim 1, comprising a recombinant biosynthetic pathway for producing at least one of putrescine, HMDA, 1,3-diaminopropane, spermidine, agmatine, cadaverine, ethylenediamine, citrulline, and ornithine.

9. The bacterial cell of claim 1, comprising
a) a genetic modification which reduces expression of proV, and at least one mutation selected from RpsG-L157* and MreB-A298V, optionally wherein the aliphatic polyamine is putrescine;
b) a genetic modification which reduces expression of proV, and mutations RpsG-L157* and MreB-A298V, optionally wherein the aliphatic polyamine is putrescine;
c) a genetic modification which reduces expression of pro V, and a mutation YgaC-R43L, optionally wherein the aliphatic polyamine is putrescine;
d) a genetic modification which reduces expression of ybeX, and at least one of proV, ptsP, cspC, and mpl, and at least one mutation selected from SspA-F83C, NusA-L152R, RpsG-L157*, YgaC-R43L, and MreB-A298V, optionally wherein the aliphatic polyamine is HMDA;
e) a genetic modification which reduces expression of ybeX and mpl, and mutations NusA-L152R and SspA-F83C, optionally wherein the aliphatic polyamine is HMDA;
f) a genetic mutation which reduces expression of ybeX and mpl, and mutations RpsG-L157* and MreB-A298V, optionally wherein the aliphatic polyamine is HMDA; or
g) a genetic mutation which reduces expression of ybeX and mpl, and mutation YgaC-R43L, optionally wherein the aliphatic polyamine is HMDA.

10. The bacterial cell of claim 1, which is of the Escherichia, Bacillus, Ralstonia, Pseudomonas or Corynebacterium genus.

11. A process for preparing a bacterial cell according to claim 1, comprising genetically modifying an E. coli cell to
a) introduce a recombinant biosynthetic pathway for producing a polyamine; and
b) knock-down or knock-out at least one endogenous gene selected from the group consisting of proV, proW, proX, cspC, ptsP, wbbK, yobF, nagC, nagA, rph, ybeX and mpl, and, optionally
c) introduce at least one mutation selected from YgaC-R43L, RpsG-L157*, MreB-A298V, MreB-N34K, MreB-E212A, MreB-I24M, MreB-H93N, NusA-L152R, NusA-M204R, SspA-F83C, SspA-V91F, MrdB-E254K, RpoD-E575A, RpoC-V401G, RpoC-V453I, RpoC-R1140C, RpoC-L120P, RpoB-R637L, RpoB-G181V, MurA-G141A, MurA-Y393S, RpsA-D160V, RpsA-D310Y, RpsA-D310G, RpsA-N313K, RpsA-N315K, RpsA-E427R, SpoT-R209H, SpoT-R467H, SpoT-R467L, SpoT-R471H, SpoT-R488C, SpoT-G530C and argG-C324A.

12. A process for improving the tolerance of an E. coli cell to at least one aliphatic polyamine selected from putrescine, HMDA, 1,3-diaminopropane, spermidine, agmatine, cadaverine, ethylenediamine, citrulline, and ornithine, comprising
a) genetically modifying the E. coli cell to knock-down or knock-out at least one endogenous gene selected from the group consisting of proV, proW, proX, cspC, ptsP, wbbK, yobF, nagC, nagA, rph, ybeX and mpl;
b) preparing a population of E. coli cells comprising one or more mutations in at least one endogenous gene selected from ygaC, rpsG, mreB, nusA, sspA, mrdA, rpoD, rpoC, rpoB, murA, rpsA, spoT, and argG; and selecting any host cell which has an improved tolerance to at least one of putrescine, HMDA, 1,3-diaminopropane, spermidine, agmatine, cadaverine, ethylenediamine, citrulline, and ornithine; or
c) both a) and b).

13. A method for producing an aliphatic polyamine, comprising culturing the bacterial cell of claim 1 in the presence of a carbon source.

14. A bacterial cell according to claim 1, comprising genetic modifications which reduce the expression of at least two endogenous genes selected from the group consisting of proV, proW, proX, cspC, ptsP, wbbK, yobF, nagC, nagA, rph, ybeX and mpl.

15. A composition comprising putrescine, HMDA, cadaverine, spermidine, agmatine, 1,3-diaminopropane, ethylenediamine, citrulline, or ornithine at a concentration of at least 10 g/L and a plurality of bacterial cells of the Escherichia genus which comprise
a) at least one genetic modification which reduces expression of an endogenous gene selected from the group consisting of proV, proW, proX, cspC, ptsP, wbbK, yobF, nagC, nagA, rph, ybeX and mpl, or a combination of any thereof;
b) a mutation in at least one of ygaC, rpsG, mreB, nusA, sspA, mrdA, rpoD, rpoC, rpoB, murA, rpsA, spoT and argG which improves the tolerance of the bacterial cell to putrescine, HMDA, cadaverine, 1,3-diaminopropane, spermidine, agmatine, ethylenediamine, citrulline, or ornithine; or
c) a combination of a) and b).

16. The bacterial cell of claim 6, which is of the Escherichia, Bacillus, Ralstonia, Pseudomonas or Corynebacterium genus, such as of the Escherichia coli species.

17. The bacterial cell of claim 6, comprising a recombinant biosynthetic pathway for producing an aliphatic polyamine.

18. The bacterial cell of claim 17, comprising a recombinant biosynthetic pathway for producing at least one of putrescine, HMDA, 1,3-diaminopropane, spermidine, agmatine, cadaverine, ethylenediamine, citrulline, and ornithine.

19. A process for preparing a bacterial cell according to claim 6, comprising genetically modifying an E. coli cell to
a) introduce a recombinant biosynthetic pathway for producing a polyamine; and
b) introduce at least one mutation selected from YgaC-R43L, RpsG-L157*, MreB-A298V, MreB-N34K, MreB-E212A, MreB-I24M, MreB-H93N, NusA-L152R, NusA-M204R, SspA-F83C, SspA-V91F, MrdB-E254K, RpoD-E575A, RpoC-V401G, RpoC-V453I, RpoC-R1140C, RpoC-L120P, RpoB-R637L, RpoB-G181V, MurA-G141A, MurA-Y393S, RpsA-D160V, RpsA-D310Y, RpsA-D310G, RpsA-N313K, RpsA-N315K, RpsA-E427R, SpoT-R209H, SpoT-R467H, SpoT-R467L, SpoT-R471H, SpoT-R488C, SpoT-G530C and argG-C324A; and, optionally
c) knock-down or knock-out at least one endogenous gene selected from the group consisting of proV, proW, proX, cspC, ptsP, wbbK, yobF, nagC, nagA, rph, ybeX and mpl.

20. A method for producing an aliphatic polyamine, comprising culturing the bacterial cell of claim 18 in the presence of a carbon source.
